(19) United States
(12) Patent Application Publication
(10) Pub. No.: US 2011/0256132 A1
(43) Pub. Date: Oct. 20, 2011

(54) MYOSTATIN BINDING PROTEINS

(76) Inventors: Claire Ashman, Hertfordshire (GB); Andrew Beaton, Hertfordshire (GB); Jonathan Henry Ellis, Hertfordshire (GB); Baijin Han, Durham, NC (US); Ian Kirby, Hertfordshire (GB); Frederick Kull, Durham, NC (US); Alan Lewis, Hertfordshire (GB); Kathryn Mason Lindley, Durham, NC (US); Martin Anibal Orecchia, Hertfordshire (GB); Ying Shen, Durham, NC (US); Paul Wilson, Hertfordshire (GB); Tian Shun Xun, Durham, NC (US); Hong Zhang, Collegeville, PA (US)

(21) Appl. No.: 13/140,841

(22) PCT Filed: Dec. 18, 2009
(86) PCT No.: PCT/EP09/67515

§ 371 Date: Jun. 20, 2011

Related U.S. Application Data
(60) Provisional application No. 61/138,980, filed on Dec. 19, 2008.

(51) Int. Cl. A61K 39/395; C07H 21/04; C12N 15/63; C12N 5/10; C12N 1/00; C12P 21/02; A61P 21/00; A61P 31/18; A61P 35/00; A61P 17/02; A61P 25/00; A61P 19/08; A61P 3/10; A61P 3/04; A61P 19/02; A61P 13/12; A61P 9/04; A61P 25/28; A61P 25/16; A61P 19/10; A61P 1/16; A61P 43/00; C07K 16/22

(52) U.S. Cl. 424/133.1; 530/389.2; 530/387.3; 530/388.23; 536/23.53; 435/320.1; 435/328; 435/335; 435/243; 424/158.1; 435/69.6

(57) ABSTRACT

Description of antigen binding proteins, such as antibodies, which bind to myostatin, polynucleotides encoding such antigen binding proteins, pharmaceutical compositions comprising said antigen binding proteins and methods of manufacture. Furthermore, description of the use of such antigen binding proteins in the treatment or prophylaxis of diseases associated with anyone or a combination of decreased muscle mass, muscle strength and muscle function.

### FIELD OF INVENTION

The present invention relates to antigen binding proteins, such as antibodies, which bind to myostatin, polynucleotides encoding such antigen binding proteins, pharmaceutical compositions comprising said antigen binding proteins and methods of manufacture. The present invention also concerns the use of such antigen binding proteins in the treatment or prophylaxis of diseases associated with any one or a combination of decreased muscle mass, muscle strength and muscle function.

### BACKGROUND OF THE INVENTION

Myostatin, also known as Growth and Differentiation Factor (GDF-8), is a member of the Transforming Growth Factor-beta (TGF-β) superfamily and is a negative regulator of muscle mass. Myostatin is highly conserved throughout evolution and the sequences of human, chicken, mouse and rat are 100% identical in the mature C-terminal domain. Myostatin is synthesised as a precursor protein that contains a signal sequence, a pro-peptide domain and a C-terminal domain. Secreted, circulating forms of myostatin include the active mature C-terminal domain and an inactive form comprising the mature C-terminal domain in a latent complex associated with the pro-peptide domain and/or other inhibitory proteins.

There are a number of different diseases, disorders and conditions that are associated with reduced muscle mass, muscle strength and muscle function. Increased exercise and better nutrition are the mainstays of current therapy for the treatment of such diseases. Unfortunately, the benefits of increased physical activity are seldom realised due to poor persistence and compliance on the part of patients. Also, exercise can be difficult, painful or impossible for some patients. Moreover there may be insufficient muscular exertion associated with exercise to produce any beneficial effect on muscle. Nutritional interventions are only effective if there are underlying dietary deficiencies and the patient has an adequate appetite. Due to these limitations, treatments for diseases associated with decreases in any one or a combination of muscle mass, muscle strength, and muscle function with more widely attainable benefits are a substantial unmet need.

Antibodies to myostatin have been described (WO 2008/030706, WO 2007/047112, WO 2007/044411, WO 2006/116269, WO 2005/094446, WO 2004/037861, WO 03/027248 and WO 94/21681). Also, Wagner et al. (Ann Neurol. (2008) 63(5): 561-71) describe no improvements in exploratory end points of muscle strength or function in adult muscular dystrophies (Becker muscular dystrophy, facioscapulohumeral dystrophy, and limb-girdle muscular dystrophy) when using one of the anti-myostatin antibodies described.

Therefore, there remains a need for more effective therapies for the treatment or prophylaxis of diseases associated with decreases in any one or a combination of muscle mass, muscle strength, and muscle function.

### SUMMARY OF THE INVENTION

The present invention provides an antigen binding protein which specifically binds to myostatin. The antigen binding protein can be used to treat or prevent a disease associated with any one or a combination of decreased muscle mass, muscle strength, and muscle function.

The present invention provides an antigen binding protein which specifically binds to myostatin and comprises CDRH3 of SEQ ID NO: 3 or a variant CDRH3.

The present invention also provides an antigen binding protein which specifically binds to myostatin and comprises the corresponding CDRH3 of the variable domain sequence of SEQ ID NO: 7, or a variant CDRH3 thereof.

The present invention also provides an antigen binding protein which specifically binds to myostatin and comprises a binding unit H3 comprising Kabat residues 95-101 of SEQ ID NO: 7, or a variant H3.

The present invention also provides an antigen binding protein which specifically binds to myostatin and comprises:
(i) a heavy chain variable region selected from SEQ ID NO: 7 or SEQ ID NO: 25; and/or a light chain variable region selected from SEQ ID NO: 8 or SEQ ID NO: 21; or a variant heavy chain variable region or light chain variable region with 75% or greater sequence identity; or
(ii) a heavy chain of SEQ ID NO: 26; and/or a light chain selected from SEQ ID NO: 27 or SEQ ID NO: 37; or a variant heavy chain or light chain with 75% or greater sequence identity.

The present invention also provides an antigen binding protein which specifically binds to myostatin and comprises:
(i) a heavy chain variable region selected from any one of SEQ ID NO: 12, 13 or 14; and/or a light chain variable region selected from any one of SEQ ID NO: 15, 16, 17, 18 or 24; or a variant heavy chain variable region or light chain variable region with 75% or greater sequence identity; or
(ii) a heavy chain selected from any one of SEQ ID NO: 28, 29, 30, 98 or 99; and/or a light chain selected from any one of SEQ ID NO: 31, 32, 33, 34 or 40; or a variant heavy chain or light chain with 75% or greater sequence identity.

The invention also provides a nucleic acid molecule which encodes an antigen binding protein as defined herein. The invention also provides an expression vector comprising a nucleic acid molecule as defined herein. The invention also provides a recombinant host cell comprising an expression vector as defined herein. The invention also provides a method for the production of an antigen binding protein as defined herein which method comprises the step of culturing a host cell as defined above and recovering the antigen binding protein. The invention also provides a pharmaceutical composition comprising an antigen binding protein thereof as defined herein and a pharmaceutically acceptable carrier.

The invention also provides a method of treating a subject afflicted with a disease which reduces muscle mass, muscle strength and/or muscle function, which method comprises the step of administering an antigen binding protein as defined herein.

The invention provides a method of treating a subject afflicted with sarcopenia, cachexia, muscle-wasting, disuse muscle atrophy, HIV, AIDS, cancer, surgery, burns, trauma or injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's syndrome, acute respiratory distress syndrome, steroid induced muscle wasting, myositis or scoliosis, which method comprises the step of administering an antigen binding protein as described herein.

The invention provides a method of increasing muscle mass, increasing muscle strength, and/or improving muscle function in a subject which method comprises the step of administering an antigen binding protein as defined herein.

The invention provides an antigen binding protein as described herein for use in the treatment of a subject afflicted with a disease which reduces any one or a combination of muscle mass, muscle strength and muscle function.

The invention provides an antigen binding protein as described herein for use in the treatment of sarcopenia, cachexia, muscle-wasting, disuse muscle atrophy, HIV, AIDS, cancer, surgery, burns, trauma or injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's muscle wasting, myositis or scoliosis.

The invention provides an antigen binding protein as described herein for use in a method of increasing muscle mass, increasing muscle strength, and/or improving syndrome, acute respiratory distress syndrome, steroid induced muscle function in a subject.

The invention provides the use of an antigen binding protein as described herein in the manufacture of a medicament for use in the treatment of a subject afflicted with a disease which reduces any one or a combination of muscle mass, muscle strength and muscle function.

The invention provides the use of an antigen binding protein as described herein in the manufacture of a medicament for use in the treatment of sarcopenia, cachexia, muscle-wasting, disuse muscle atrophy, HIV, AIDS, cancer, surgery, burns, trauma or injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's muscle wasting, myositis or scoliosis.

The invention provides the use of an antigen binding protein as described herein in the manufacture of a medicament for use in a method of increasing muscle mass, increasing muscle strength, and/or improving syndrome, acute respiratory distress syndrome, steroid induced muscle function in a subject.

### BRIEF DESCRIPTION OF THE FIGURES

FIG. 1 shows the LC/MS analysis for purified mature myostatin: predicted Molecular Weight (MW) 12406.25 Da, observed MW 24793.98 Da, which indicates a dimerised molecule with nine pairs of disulphide bonds, matching the predicted myostatin monomer with nine cysteine residues.

FIG. 2 shows a 4-12% NuPAGE Bis-Tris gel with MOPS buffer. Lane 1: mature myostatin reduced with DTT. Lane 2: mature myostatin non-reduced without DTT. Lane 3: Mark 12 protein standard.

FIG. 3A shows dose response curves demonstrating myostatin (R&D Systems and in-house myostatin species) induced activation of cell signalling, resulting in luciferase expression after 6 hours in a dose dependent manner in A204 cells. FIG. 3B shows dose response curves demonstrating in-house myostatin induced activation of cell signalling, resulting in luciferase expression in a dose dependent manner in A204 cells, on different test occasions as represented by data obtained on different days.

FIG. 4 shows 10B3 binding to mature myostatin, latent complex and mature myostatin released from latent complex by ELISA.

FIG. 5 shows inhibition of myostatin binding to ActRIIb by 10B3 and 10B3 chimera.

FIG. 6 shows the 10B3 and 10B3 chimera inhibition of myostatin-induced activation of cell signalling, resulting in decreased luciferase expression in A204 cells.

FIG. 7 shows the in vivo effects of 10B3 on body weight (A) and lean mass (B) in mice.

FIG. 8 shows the in vivo effects of 10B3 on muscle mass in gastrocnemius (A), quadriceps (B), and extensor digitorum longus (EDL) (C) in mice.

FIG. 9 shows the ex vivo effects of 10B3 on muscle contractility in EDL, showing tetanic force (A) and tetanic force corrected by muscle mass (B).

FIG. 10A shows the binding of humanised anti-myostatin antibody variants (in CHOK1 supernatants) and 10B3C to myostatin by ELISA. FIG. 10B is derived from FIG. 10A and displays antibodies containing the H2 and/or L2 chains and 10B3 chimera.

FIG. 11 shows the binding of purified H0L0, H1L2 and H2L2 humanised anti-myostatin antibody variants and 10B3C to myostatin by ELISA.

FIG. 12 shows 10B3, 10B3C, H0L0 and H2L2 inhibition of myostatin-induced activation of cell signalling, resulting in luciferase expression in A204 cells.

FIG. 13 shows the binding of purified H2L2-N54D, H2L2-N54Q, H2L2-C91S, H2L2-N54D-C91S and H2L2-N54Q-C91S humanised anti-myostatin antibody variants, H2L2 and 10B3C(HCLC) to myostatin by ELISA.

FIG. 14 shows the binding of purified H2L2-N54Q, H2L2-C91S, H2L2-N54Q-C91S humanised anti-myostatin antibody variants, H2L2, H0L0 and 10B3C (HCLC) to myostatin by ELISA.

FIG. 15 shows the H2L2-N54Q, H2L2-C91S, H2L2-N54Q-C91S humanised anti-myostatin antibody variants, H0L0, H2L2 and 10B3C inhibition of myostatin-induced activation of cell signalling, resulting in luciferase expression in A204 cells.

FIG. 16 shows binding of the H2L2 humanised anti-myostatin antibody to myostatin following treatment of the antibody with or without ammonium bicarbonate which can induce deamidation of the antibody.

FIG. 17 shows binding of the H2L2-N54Q humanised anti-myostatin antibody variant to myostatin following treatment of the antibody with or without ammonium bicarbonate which can induce deamidation of the antibody.

FIG. 18 shows binding of the H2L2-C91S humanised anti-myostatin antibody variant to myostatin following treatment of the antibody with or without ammonium bicarbonate which can induce deamidation of the antibody.

FIG. 19 shows binding of the H2L2-N54Q-C91S humanised anti-myostatin antibody variant to myostatin following treatment of the antibody with or without ammonium bicarbonate which can induce deamidation of the antibody.

FIG. 20 shows binding of the H0L0 humanised anti-myostatin antibody to myostatin following treatment of the antibody with or without ammonium bicarbonate which can induce deamidation of the antibody.

FIG. 21 shows the binding activity in the myostatin capture ELISA of the eleven affinity purified CDRH3 variants; and H2L2-C91S, H0L0, HcLc (10B3 chimera) and a negative control monoclonal antibody which were used as control antibodies.

FIG. 22 shows the binding activity in the myostatin binding ELISA of the five affinity purified CDRH2 variants; and H2L2-C91S_F100G_Y, H2L2-C91S, HcLc (10B3 chimera) and a negative control monoclonal antibody which were used as control antibodies.

FIG. 23 shows the effect of 10B3 and control antibody treatment on body weight in C-26 tumour bearing mice from day 0 to day 25.

FIG. 24 shows the effect of 10B3 and control antibody treatment on total body fat (A), epididymal fat pad (B), and lean mass (C), in C-26 tumour bearing mice.

FIG. 25 shows the effect of 10B3 and control antibody treatment on lower limb muscle strength, which was measured by the contraction force upon the electrical stimulation of sciatic nerve on the mid thigh in C-26 tumour bearing mice.

FIG. 26 shows the effect of 10B3 and control antibody treatment in sham operated and tenotomy surgery on mouse tibialis anterior (TA) muscle.

### DETAILED DESCRIPTION OF THE INVENTION

The present invention provides an antigen binding protein which specifically binds to myostatin, for example homodimeric mature myostatin. The antigen binding protein may bind to and neutralise myostatin, for example human myostatin. The antigen binding protein may be an antibody, for example a monoclonal antibody.

Myostatin and GDF-8 both refer to any one of the full-length unprocessed precursor form of myostatin; mature myostatin which results from post-translational cleavage of the C-terminal domain, in latent and non-latent (active) forms. The term myostatin also refers to any fragments and variants of myostatin that retain one or more biological activities associated with myostatin.

The full-length unprocessed precursor form of myostatin comprises pro-peptide and the C-terminal domain which forms the mature protein, with or without a signal sequence. Myostatin pro-peptide plus C-terminal domain is also known as polyprotein. The myostatin precursor may be present as a monomer or homodimer.

Mature myostatin is the protein that is cleaved from the C-terminus of the myostatin precursor protein, also known as the C-terminal domain. Mature myostatin may be present as a monomer, homodimer, or in a myostatin latent complex. Depending on conditions, mature myostatin may establish equilibrium between a combination of these different forms. The mature C-terminal domain sequences of human, chicken, mouse and rat myostatin are 100% identical (see for example SEQ ID NO: 104). In one embodiment, the antigen binding protein of the invention binds to homodimeric, mature myostatin shown in SEQ ID NO: 104.

Myostatin pro-peptide is the polypeptide that is cleaved from the N-terminal domain of the myostatin precursor protein following cleavage of the signal sequence. Pro-peptide is also known as latency-associated peptide (LAP). Myostatin pro-peptide is capable of non-covalently binding to the pro-peptide binding domain on mature myostatin. An example of the human pro-peptide myostatin sequence is provided in SEQ NO: 108.

Myostatin latent complex is a complex of proteins formed between mature myostatin and myostatin pro-peptide or other myostatin-binding proteins. For example, two myostatin pro-peptide molecules can associate with two molecules of mature myostatin to form an inactive tetrameric latent complex. The myostatin latent complex may include other myostatin-binding proteins in place of or in addition to one or both of the myostatin pro-peptides. Examples of other myostatin-binding proteins include follistatin, follistatin-related gene (FLRG) and Growth and Differentiation Factor-Associated Serum Protein 1 (GASP-1).

The myostatin antigen binding protein may bind to any one or any combination of precursor, mature, monomeric, dimeric, latent and active forms of myostatin. The antigen binding protein may bind mature myostatin in its monomeric and/or dimeric forms. The antigen binding protein may or may not bind myostatin when it is in a complex with pro-peptide and/or follistatin. Alternatively the antigen binding protein may or may not bind myostatin when it is in a complex with follistatin-related gene (FLRG) and/or Growth and Differentiation Factor-Associated Serum Protein 1 (GASP-1). For example, the antigen binding protein binds to mature dimeric myostatin.

The term “antigen binding protein” as used herein refers to antibodies, antibody fragments and other protein constructs, such as domains, which are capable of binding to myostatin.

The term “antibody” is used herein in the broadest sense to refer to molecules with an immunoglobulin-like domain and includes monoclonal, recombinant, polyclonal, chimeric, humanised, bispecific and heteroconjugate antibodies; a single variable domain, a domain antibody, antigen binding fragments, immunologically effective fragments, single chain Fv, diabodies, Tandabs™, etc (for a summary of alternative “antibody” formats see Holliger and Hudson, Nature Biotechnology, 2005, Vol 23, No. 9, 1126-1136).

The phrase “single variable domain” refers to an antigen binding protein variable domain (for example, V_{H}, V_{HH}, V_{L}) that specifically binds an antigen or epitope independently of a different variable region or domain.

A “domain antibody” or “dAb” may be considered the same as a “single variable domain” which is capable of binding to an antigen. A single variable domain may be a human antibody variable domain, but also includes single antibody variable domains from other species such as rodent (for example, as disclosed in WO 00/29004), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are considered to be “domain antibodies”. As used herein V_{H} includes camelid V_{HH} domains.

As used herein the term “domain” refers to a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A domain can bind an antigen or epitope independently of a different variable region or domain.

An antigen binding fragment may be provided by means of arrangement of one or more CDRs on non-antibody protein scaffolds such as a domain. A non-antibody protein scaffold or domain is one that has been subjected to protein engineering in order to obtain binding to a ligand other than its natural ligand, for example a domain which is a derivative of a scaffold selected from: CTLA-4 (Evibody); lipocalin; Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001).

Lipocalins are a family of extracellular proteins which transport small hydrophobic molecules such as steroids, bilins, retinoids and lipids. They have a rigid β-sheet secondary structure with a number of loops at the open end of the canonical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to an antigen. The domain consists of a three-helical bundle of approximately 58 amino acids. Libraries have been generated by randomisation of surface residues. For further details see Protein Eng. Des. Sel. 17, 455-462 (2004) and EP1641818A1.

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences, such as one or more CDRs, in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by: randomising residues in the first α-helix and a β-turn of each repeat; or insertion of peptide sequences, such as one or more CDRs. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges; examples of microproteins include KalataB1 and conotoxin and knottins. The microproteins have a loop which can be engineered to include up to 25 amino acids without affecting the overall fold of the microprotein. For further details of engineered knottin domains, see WO2008098796.

Other binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Binding domains of the present invention could be derived from any of these alternative protein domains and any combination of the CDRs of the present invention grafted onto the domain.

An antigen binding fragment or an immunologically effective fragment may comprise partial heavy or light chain variable sequences. Fragments are at least 5, 6, 8 or 10 amino acids in length. Alternatively the fragments are at least 15, at least 20, at least 50, at least 75, or at least 100 amino acids in length.

The term “specifically binds” as used throughout the present specification in relation to antigen binding proteins means that the antigen binding protein binds to myostatin with no or insignificant binding to other (for example, unrelated) proteins. The term however does not exclude the fact that the antigen binding proteins may also be cross-reactive with closely related molecules (for example, Growth and Differentiation Factor-11). The antigen binding proteins described herein may bind to myostatin with at least 2, 5, 10, 50, 100, or 1000 fold greater affinity than they bind to closely related molecules, such as GDF-11.

The binding affinity or equilibrium dissociation constant (K_{D}) of the antigen binding protein-myostatin interaction may be 100 nM or less, 10 nM or less, 2 nM or less or 1 nM or less. Alternatively the K_{D} may be between 5 and 10 nM; or between 1 and 2 nM. The K_{D} may be between 1 pM and 500 pM; or between 500 pM and 1 nM. The binding affinity of the antigen binding protein is determined by the association rate constant (kₐ) and the dissociation rate constant (k_{d}) (K_{D}=k_{d}/kₐ). The binding affinity may be measured by BIAcore™, for example by antigen capture with myostatin coupled onto a CM5 chip by primary amine coupling and antibody capture onto this surface. The BIAcore™ method described in Example 2.3 may be used to measure binding affinity. Alternatively, the binding affinity can be measured by FORTEbio, for example by antigen capture with myostatin coupled onto a CM5 needle by primary amine coupling and antibody capture onto this surface. The FORTEbio method described in Example 5.1 may be used to measure binding affinity. However, due to the nature of the binding of the antigen binding protein of the invention to myostatin, binding affinity may be used for ranking purposes.

The k_{d} may be 1×10⁻³ s⁻¹ or less, 1×10⁻⁴ s⁻¹ or less, or 1×10⁻⁵ s⁻¹ or less. The k_{d} may be between 1×10⁻⁵ s⁻¹ and 1×10⁻⁴ s⁻¹; or between 1×10⁻⁴ s⁻¹ and 1×10⁻³ s⁻¹. A slow k_{d} may result in a slow dissociation of the antigen binding protein-ligand complex and improved neutralisation of the ligand.

The term “neutralises” as used throughout the present specification means that the biological activity of myostatin is reduced in the presence of an antigen binding protein as described herein in comparison to the activity of myostatin in the absence of the antigen binding protein, in vitro or in vivo. Neutralisation may be due to one or more of blocking myostatin binding to its receptor, preventing myostatin from activating its receptor, down regulating myostatin or its receptor, or affecting effector functionality. Neutralisation may be due to blocking myostatin binding to its receptor and therefore preventing myostatin from activating its receptor.

Myostatin activity includes one or more of the growth, regulatory and morphogenetic activities associated with active myostatin, for example modulating muscle mass, muscle strength and muscle function. Further activities associated with active myostatin may include modulation of muscle fibre number, muscle fibre size, muscle regeneration, muscle fibrosis, the proliferation rate of myoblasts, myogenic differentiation; activation of satellite cells, proliferation of satellite cells, self renewal of satellite cells; synthesis or catabolism of proteins involved in muscle growth and function. The muscle may be skeletal muscle.

The reduction or inhibition in biological activity may be partial or total. A neutralising antigen binding protein may neutralise the activity of myostatin by at least 20%, 30% 40%, 50%, 55%, 60%, 65%, 70%, 75%, 80%, 82%, 84%, 86%, 88%, 90%, 92%, 94%, 95%, 96%, 97%, 98%, 99% or 100% relative to myostatin activity in the absence of the antigen binding protein. In functional assays (such as the neutralisation assays described below), IC₅₀ is the concentration that reduces a biological response by 50% of its maximum.

Neutralisation may be determined or measured using one or more assays known to the skilled person or as described herein. For example, antigen binding protein binding to myostatin can be assessed in a sandwich ELISA, by BIAcore™, FMAT, FORTEbio™, or similar in vitro assays such as surface Plasmon resonance.

An ELISA-based receptor binding assay can be used to determine the neutralising activity of the antigen binding protein by measuring myostatin binding to soluble ActRIIb receptor immobilised on a plate in the presence of the antigen binding protein (for more detail see Example 2.5). The receptor neutralisation assay is a sensitive method which is available for differentiating molecules with IC50s lower than 1 nM on the basis of potency. It is, however, itself sensitive to the precise concentration of binding-competent biotinylated myostatin. Hence, IC50 values in the range of from 0.1 nM to 5 nM may be obtained, for example, from 0.1 nM to 3 nM, or from 0.1 nM to 2 nM, or from 0.1 nM to 1 nM.

Alternatively, a cell-based receptor binding assay can be used to determine the neutralising activity of the antigen binding protein by measuring inhibition of receptor binding, downstream signalling and gene activation. For example, neutralising antigen binding proteins can be identified by their ability to inhibit myostatin-induced luciferase activity in Rhabdomyosarcoma cells (A204) transfected with a construct encoding a luciferase gene under the control of a PAI-1 specific promoter, also known as the myostatin responsive reporter gene assay (for more detail see Example 1.2).

In vivo neutralisation may be determined using a number of different assays in animals which demonstrate changes in any one or a combination of muscle mass, muscle strength, and muscle function. For example, body weight, muscle mass (such as lean muscle mass), muscle contractility (for example tetanic force), grip strength, an animal's ability to suspend itself, and swim test, can be used in isolation or in any combination to assess the neutralising activity of the myostatin antigen binding protein. For example the muscle mass of the following muscles may be determined: gastrocnemius, quadriceps, triceps, extensor digitorum longus (EDL), tibialis anterior (TA) and soleus.

It will be apparent to those skilled in the art that the term “derived” is intended to define not only the source in the sense of it being the physical origin for the material but also to define material which is structurally identical to the material but which does not originate from the reference source. Thus “residues found in the donor antibody” need not necessarily have been purified from the donor antibody.

By isolated it is intended that the molecule, such as an antigen binding protein, is removed from the environment in which it may be found in nature. For example, the molecule may be purified away from substances with which it would normally exist in nature. For example, the antigen binding protein can be purified to at least 95%, 96%, 97%, 98% or 99%, or greater with respect to a culture media containing the antigen binding protein.

A “chimeric antibody” refers to a type of engineered antibody which contains a naturally-occurring variable region (light chain and heavy chains) derived from a donor antibody in association with light and heavy chain constant regions derived from an acceptor antibody.

A “humanised antibody” refers to a type of engineered antibody having one or more of its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl. Acad Sci USA, 86:10029-10032 (1989), Hodgson et al. Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies, see for example EP-A-0239400 and EP-A-054951.

The term “donor antibody” refers to an antibody which contributes the amino acid sequences of its variable regions, one or more CDRs, or other functional fragments or analogs thereof to a first immunoglobulin partner. The donor therefore provides the altered immunoglobulin coding region and resulting expressed altered antibody with the antigenic specificity and neutralising activity characteristic of the donor antibody.

The term “acceptor antibody” refers to an antibody which is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention, unless otherwise specified. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person. Therefore, the term “corresponding CDR” is used herein to refer to a CDR sequence using any numbering convention, for example those set out in Table 1.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat | Chothia | AbM | Contact | binding |
| | CDR | CDR | CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

As used herein, the term “antigen binding site” refers to a site on an antigen binding protein which is capable of specifically binding to an antigen. This may be a single domain (for example, an epitope-binding domain), or single-chain Fv (ScFv) domains or it may be paired V_{H}/V_{L} domains as can be found on a standard antibody.

The term “epitope” as used herein refers to that portion of the antigen that makes contact with a particular binding domain of the antigen binding protein. An epitope may be linear, comprising an essentially linear amino acid sequence from the antigen. Alternatively, an epitope may be conformational or discontinuous. For example, a conformational epitope comprises amino acid residues which require an element of structural constraint. A discontinuous epitope comprises amino acid residues that are separated by other sequences, i.e. not in a continuous sequence in the antigen's primary sequence. In the context of the antigen's tertiary and quaternary structure, the residues of a discontinuous epitope are near enough to each other to be bound by an antigen binding protein.

For nucleotide and amino acid sequences, the term “identical” or “sequence identity” indicates the degree of identity between two nucleic acid or two amino acid sequences, and if required when optimally aligned and compared with appropriate insertions or deletions.

The percent identity between two sequences is a function of the number of identical positions shared by the sequences (i.e., % identity=number of identical positions/total number of positions times 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

In one method, a polynucleotide sequence may be identical to a reference polynucleotide sequence as described herein (see for example SEQ ID NO: 41-55), that is be 100% identical, or it may include up to a certain integer number of nucleotide alterations as compared to the reference sequence, such as at least 50, 60, 70, 75, 80, 85, 90, 95, 98, or 99% identical. Such alterations are selected from at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference polynucleotide sequence as described herein (see for example SEQ ID NO: 41-55), by the numerical percent of the respective percent identity (divided by 100) and subtracting that product from said total number of nucleotides in the reference polynucleotide sequence as described herein (see for example SEQ ID NO: 41-55), or:

nₙ≦xₙ−(xₙ•y),

wherein nₙ is the number of nucleotide alterations, xₙ is the total number of nucleotides in the reference polynucleotide sequence as described herein (see for example SEQ ID NO: 41-55), and y is 0.50 for 50%, 0.60 for 60%, 0.70 for 70%, 0.75 for 75%, 0.80 for 80%, 0.85 for 85%, 0.90 for 90%, 0.95 for 95%, 0.98 for 98%, 0.99 for 99% or 1.00 for 100%, • is the symbol for the multiplication operator, and wherein any non-integer product of xₙ and y is rounded down to the nearest integer prior to subtracting it from xₙ.

Similarly, a polypeptide sequence may be identical to a polypeptide reference sequence as described herein (see for example SEQ ID NO: 7-40, 98 or 99) that is be 100% identical, or it may include up to a certain integer number of amino acid alterations as compared to the reference sequence such that the % identity is less than 100%, such as at least 50, 60, 70, 75, 80, 85, 90, 95, 98, or 99% identical. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by the polypeptide reference sequence as described herein (see for example SEQ ID NO: 7-40, 98 or 99) by the numerical percent of the respective percent identity (divided by 100) and then subtracting that product from said total number of amino acids in the polypeptide reference sequence as described herein (see for example SEQ ID NO: 7-40 or 82-108, 98 or 99), or:

nₐ≦xₐ−(xₐ•y),

wherein nₐ is the number of amino acid alterations, xₐ is the total number of amino acids in the reference polypeptide sequence as described herein (see for example SEQ ID NO: 7-40, 98 or 99), and y is, 0.50 for 50%, 0.60 for 60%, 0.70 for 70%, 0.75 for 75%, 0.80 for 80%, 0.85 for 85%, 0.90 for 90%, 0.95 for 95%, 0.98 for 98%, 0.99 for 99%, or 1.00 for 100%, • is the symbol for the multiplication operator, and wherein any non-integer product of xₐ and y is rounded down to the nearest integer prior to subtracting it from xₐ.

The % identity may be determined across the full length of the sequence, or any fragments thereof; and with or without any insertions or deletions.

The terms “peptide”, “polypeptide” and “protein” each refers to a molecule comprising two or more amino acid residues. A peptide may be monomeric or polymeric.

It is well recognised in the art that certain amino acid substitutions are regarded as being “conservative”. Amino acids are divided into groups based on common side-chain properties and substitutions within groups that maintain all or substantially all of the binding affinity of the antigen binding protein are regarded as conservative substitutions, see Table 2 below:

TABLE 2

| Side chain | Members |
|---|---|
| Hydrophobic | met, ala, val, leu, ile |
| Neutral hydrophilic | cys, ser, thr |
| Acidic | asp, glu |
| Basic | asn, gln, his, lys, arg |
| Residues that influence chain orientation | gly, pro |
| Aromatic | trp, tyr, phe |

The present invention provides an antigen binding protein which binds to myostatin and comprises CDRH3 of SEQ ID NO: 3; or a variant CDRH3 thereof (for example any one of SEQ ID NOs: 82-92, or 110). The antigen binding protein may also neutralise myostatin activity.

The present invention also provides an antigen binding protein which binds to myostatin and comprises CDRH2 of SEQ ID NO: 2; or a variant CDRH2 thereof (for example any one of SEQ ID NOs: 93-97). The antigen binding protein may also neutralise myostatin activity.

The antigen binding protein may further comprise in addition to the CDRH3 or CDRH2 sequences described above, one or more CDRs, or all CDRs, in any combination, selected from: CDRH1 (SEQ ID NO: 1), CDRH2 (SEQ ID NO: 2), CDRL1 (SEQ ID NO: 4), CDRL2 (SEQ ID NO: 5), and CDRL3 (SEQ ID NO: 6 or 109); or a variant thereof (for example any one of CDRH2 variants SEQ ID NOs: 93-97).

For example, the antigen binding protein may comprise CDRH3 (SEQ ID NO: 3) and CDRH1 (SEQ ID NO: 1), or variants thereof (for example any one of CDRH3 variants 82-92, or 110). The antigen binding protein may comprise CDRH3 (SEQ ID NO: 3) and CDRH2 (SEQ ID NO: 2), or variants thereof (for example any one of CDRH3 variants SEQ ID NOs: 82-92, or 110; or any one of CDRH2 variants SEQ ID NOs: 93-97). The antigen binding protein may comprise CDRH1 (SEQ ID NO: 1) and CDRH2 (SEQ ID NO: 2), and CDRH3 (SEQ ID NO: 3), or variants thereof (for example any one of CDRH3 variants SEQ ID NOs: 82-92, or 110; or any one of CDRH2 variants SEQ ID NOs: 93-97).

The antigen binding protein may comprise CDRL1 (SEQ ID NO: 4) and CDRL2 (SEQ ID NO: 5), or variants thereof. The antigen binding protein may comprise CDRL2 (SEQ ID NO: 5) and CDRL3 (SEQ ID NO: 6 or 109), or variants thereof. The antigen binding protein may comprise CDRL1 (SEQ ID NO: 4), CDRL2 (SEQ ID NO: 5) and CDRL3 (SEQ ID NO: 6 or 109), or variants thereof.

The antigen binding protein may comprise CDRH3 (SEQ ID NO: 3) and CDRL3 (SEQ ID NO: 6 or 109), or variants thereof (for example any one of CDRH3 variants SEQ ID NOs: 82-92, or 110). The antigen binding protein may comprise CDRH3 (SEQ ID NO: 3), CDRH2 (SEQ ID NO: 2) and CDRL3 (SEQ ID NO: 6 or 109), or variants thereof (for example any one of CDRH3 variants SEQ ID NOs: 82-92, or 110; or any one of CDRH2 variants SEQ ID NOs: 93-97). The antigen binding protein may comprise CDRH3 (SEQ ID NO: 3), CDRH2 (SEQ ID NO: 2), CDRL2 (SEQ ID NO: 5) and CDRL3 (SEQ ID NO: 6 or 109), or variants thereof (for example any one of CDRH3 variants SEQ ID NOs: 82-92, or 110; or any one of CDRH2 variants SEQ ID NOs: 93-97).

The antigen binding protein may comprise CDRH1 (SEQ ID NO: 1), CDRH2 (SEQ ID NO: 2), CDRH3 (SEQ ID NO: 3), CDRL1 (SEQ ID NO: 4), CDRL2 (SEQ ID NO: 5) and CDRL3 (SEQ ID NO: 6). Alternatively, variant CDRs may be present, such as any one of CDRH3 variants SEQ ID NOs: 82-92, or 110; or any one of CDRH2 variants SEQ ID NOs: 93-97; or CDRH3 variant SEQ ID NO: 109. For example, the antigen binding protein may comprise CDRH1 (SEQ ID NO: 1), CDRH2 (SEQ ID NO: 95), CDRH3 (SEQ ID NO: 90), CDRL1 (SEQ ID NO: 4), CDRL2 (SEQ ID NO: 5) and CDRL3 (SEQ ID NO: 109).

The present invention also provides an antigen binding protein which binds to myostatin and comprises the corresponding CDRH3 of the variable domain sequence of SEQ ID NO: 7, or a variant CDRH3 thereof. The antigen binding protein may also neutralise myostatin activity. The antigen binding protein may be a chimeric or a humanised antibody.

The antigen binding protein may further comprise one or more, or all of the corresponding CDRs selected from the variable domain sequence of SEQ ID NO: 7 or SEQ ID NO: 8, or a variant CDR thereof.

For example, the antigen binding protein may comprise corresponding CDRH3 and corresponding CDRH1, or variants thereof. The antigen binding protein may comprise corresponding CDRH3 and corresponding CDRH2, or variants thereof. The antigen binding protein may comprise corresponding CDRH1, corresponding CDRH2, and corresponding CDRH3; or variants thereof.

The antigen binding protein may comprise corresponding CDRL1 and corresponding CDRL2, or variants thereof. The antigen binding protein may comprise corresponding CDRL2 and corresponding CDRL3, or variants thereof. The antigen binding protein may comprise corresponding CDRL1, corresponding CDRL2 and corresponding CDRL3, or variants thereof.

The antigen binding protein may comprise corresponding CDRH3 and corresponding CDRL3, or variants thereof. The antigen binding protein may comprise corresponding CDRH3, corresponding CDRH2 and corresponding CDRL3, or variants thereof. The antigen binding protein may comprise corresponding CDRH3, corresponding CDRH2, corresponding CDRL2 and corresponding CDRL3, or variants thereof.

The antigen binding protein may comprise corresponding CDRH1, corresponding CDRH2, corresponding CDRH3, corresponding CDRL1, corresponding CDRL2 and corresponding CDRL3, or variants thereof.

The corresponding CDRs can be defined by reference to Kabat (1987), Chothia (1989), AbM or contact methods. One definition of each of the methods can be found at Table 1 and can be applied to the reference heavy chain variable domain SEQ ID NO: 7 and the reference light chain variable domain SEQ ID NO: 8 to determine the corresponding CDR.

The present invention also provides an antigen binding protein which binds to myostatin, and comprises a binding unit H3 comprising Kabat residues 95-101 of SEQ ID NO: 7, or a variant H3. The antigen binding protein may also neutralise myostatin.

The antigen binding protein may further comprise one or more or all binding units selected from: H1 comprising Kabat residues 31-32 of SEQ ID NO: 7, H2 comprising Kabat residues 52-56 of SEQ ID NO: 7, L1 comprising Kabat residues 30-34 of SEQ ID NO: 8, L2 comprising Kabat residues 50-55 of SEQ ID NO: 8 and L3 comprising Kabat residues 89-96 of SEQ ID NO: 8; or a variant binding unit.

For example, the antigen binding protein may comprise a binding unit H3 and a binding unit H1, or variants thereof. The antigen binding protein may comprise a binding unit H3 and a binding unit H2, or variants thereof. The antigen binding protein may comprise a binding unit H1, a binding unit H2, and a binding unit H3; or variants thereof.

The antigen binding protein may comprise a binding unit L1 and a binding unit L2, or variants thereof. The antigen binding protein may comprise a binding unit L2 and a binding unit L3, or variants thereof. The antigen binding protein may comprise a binding unit L1, a binding unit L2, and a binding unit L3; or variants thereof.

The antigen binding protein may comprise a binding unit H3 and a binding unit L3, or variants thereof. The antigen binding protein may comprise a binding unit H3, a binding unit H2, and a binding unit L3; or variants thereof. The antigen binding protein may comprise a binding unit H3, a binding unit H2, a binding unit L2, and a binding unit L3; or variants thereof.

The antigen binding protein may comprise a binding unit H1, a binding unit H2, a binding unit H3, a binding unit L1, a binding unit L2, and a binding unit L3; or variants thereof.

A CDR variant or variant binding unit includes an amino acid sequence modified by at least one amino acid, wherein said modification can be chemical or a partial alteration of the amino acid sequence (for example by no more than 10 amino acids), which modification permits the variant to retain the biological characteristics of the unmodified sequence. For example, the variant is a functional variant which binds to myostatin. A partial alteration of the CDR amino acid sequence may be by deletion or substitution of one to several amino acids, or by addition or insertion of one to several amino acids, or by a combination thereof (for example by no more than 10 amino acids). The CDR variant or binding unit variant may contain 1, 2, 3, 4, 5 or 6 amino acid substitutions, additions or deletions, in any combination, in the amino acid sequence. The CDR variant or binding unit variant may contain 1, 2 or 3 amino acid substitutions, insertions or deletions, in any combination, in the amino acid sequence. The substitutions in amino acid residues may be conservative substitutions, for example, substituting one hydrophobic amino acid for an alternative hydrophobic amino acid. For example leucine may be substituted with valine, or isoleucine.

The CDRs L1, L2, L3, H1 and H2 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

Examples of CDR canonicals, where the amino acid before the Kabat number is the original amino acid sequence of SEQ ID NO: 14 or 24 and the amino acid sequence at the end of the Kabat number is the substituted amino acid, include:

CDRH1 canonicals: Y32I, Y32H, Y32F, Y32T, Y32N, Y32C, Y32E, Y32D, F33Y, F33A, F33W, F33G, F33T, F33L, F33V, M34I, M34V, M34W, H35E, H35N, H35Q, H35S, H35Y, H35T;
CDRH2 canonicals: N50R, N50E, N50W, N50Y, N50G, N50Q, N50V, N50L, N50K, N50A, I51L, I51V, I51T, I51S, I51N, Y52D, Y52L, Y52N, Y52S, Y53A, Y53G, Y53S, Y53K, Y53T, Y53N, N54S, N54T, N54K, N54D, N54G, V56Y, V56R, V56E, V56D, V56G, V56S, V56A, N58K, N58T, N58S, N58D, N58R, N58G, N58F, N58Y;
CDRH3 canonicals: V102Y, V102H, V102I, V102S, V102D, V102G;
CDRL1 canonicals: D28N, D28S, D28E, D28T, I29V, N30D, N30L, N30Y, N30V, N30I, N30S, N30F, N30H, N30G, N30T, S31N, S31T, S31K, S31G, Y32F, Y32N, Y32A, Y32H, Y32S, Y32R, L33M, L33V, L33I, L33F, S34A, S34G, S34N, S34H, S34V, S34F;
CDRL2 canonicals: A51T, A51G, A51V;
CDRL3 canonicals: L89Q, L89S, L89G, L89F, Q90N, Q90H, S91N, S91F, S91G, S91R, S91D, S91H, S91T, S91Y, S91V, D92N, D92Y, D92W, D92T, D92S, D92R, D92Q, D92H, D92A, E93N, E93G, E93H, E93T, E93S, E93R, E93A, F94D, F94Y, F94T, F94V, F94L, F94H, F94N, F94I, F94W, F94P, F94S, L96P, L96Y, L96R, L96I, L96W, L96F.

There may be multiple variant CDR canonical positions per CDR, per corresponding CDR, per binding unit, per heavy or light chain variable region, per heavy or light chain, and per antigen binding protein, and therefore any combination of substitution may be present in the antigen binding protein of the invention, provided that the canonical structure of the CDR is maintained.

Other examples of CDR variants or variant binding units include (using the Kabat numbering scheme, where the amino acid before the Kabat number is the original amino acid sequence of SEQ ID NO: 14 or 24 and the amino acid sequence at the end of the Kabat number is the substituted amino acid):

### H2: G55D, G55L, G55S, G55T, G55V;

### H3: Y96L, G99D, G99S, G100A_K, P100B_F, P100B_I, W100E_F, F100G_N, F100G_S, F100G_Y, V102N, V102S;

### L3: C91S.

For example an antigen binding protein of the invention which binds to myostatin may comprise CDRH3 of SEQ ID NO: 90. The antigen binding protein may further comprise CDRH2 of any one of SEQ ID NO: 93-97. In particular, the CDRH2 may be SEQ ID NO: 95. The antigen binding protein may also comprise CDRL3 of SEQ ID NO: 109. The antigen binding protein may further comprise any one or a combination or all of CDRH1 (SEQ ID NO: 1), CDRL1 (SEQ ID NO: 4), and CDRL2 (SEQ ID NO: 5). The antigen binding protein may also neutralise myostatin activity.

The antigen binding protein comprising the CDRs, corresponding CDRs, variant CDRs, binding units or variant binding units described, may display a potency for binding to myostatin, as demonstrated by EC50, of within 10 fold, or within 5 fold of the potency demonstrated by 10B3 or 10B3 chimera (heavy chain: SEQ ID NO: 7 or 25, light chain: SEQ ID NO: 8). Potency for binding to myostatin, as demonstrated by EC50, may be carried out by an ELISA assay.

The antigen binding protein may or may not have a substitution at amino acid Kabat position 54 of the heavy chain from asparagine (N) to aspartate (D) or glutamine (Q). The antigen binding protein variant may or may not have a substitution at amino acid position 91 of the light chain from cysteine (C) to serine (S). For example, the antigen binding protein has a serine (S) residue at position 91 of the light chain and an asparagine (N) at position 54 of the heavy chain.

The antigen binding protein variable heavy chain may have a serine (S) or Threonine (T) amino acid residue at position 28; and/or a threonine (T) or glutamine (O) amino acid residue at position 105. The antigen binding protein variable light chain may have an arginine (R) or glycine (G) amino acid residue at position 16; and/or a tyrosine (Y) or phenylalanine (F) amino acid residue at position 71; and/or an alanine (A) or glutamine (Q) amino acid residue at position 100. For example, the antigen binding protein may comprise serine (S) at position 28, glutamine (Q) at position 105 of the variable heavy chain; and/or glycine (G) at position 16, tyrosine (Y) at position 71, and glutamine (Q) at position 100 of the variable light chain.

As discussed above, the particular canonical structure class of a CDR is defined by both the length of the CDR and by the loop packing, determined by residues located at key positions in both the CDRs and the framework regions. Thus in addition to the CDRs listed in SEQ ID NO: 1-3, variant CDRs listed in SEQ ID NO: 82-97 and SEQ ID NO 109, corresponding CDRs, binding units, or variants thereof, the canonical framework residues of an antigen binding protein of the invention may include (using Kabat numbering):

Heavy chain: V, I or G at position 2; L or V at position 4; L, I, M or V at position 20; C at position 22; T, A, V, G or S at position 24; G at position 26; I, F, L or S at position 29; W at position 36; W or Y at position 47; I, M, V or L at position 48; I, L, F, M or V at position 69; A, L, V, Y or F at position 78; L or M at position 80; Y or F at position 90; C at position 92; and/or R, K, G, S, H or N at position 94; and/or

Light chain: I, L or V at position 2; V, Q, L or E at position 3; M or L at position 4; C at position 23; W at position 35; Y, L or F at position 36; S, L, R or V at position 46; Y, H, F or K at position 49; Y or F at position 71; C at position 88; and/or Fat position 98.

Any one, any combination, or all of the framework positions described above may be present in the antigen binding protein of the invention. There may be multiple variant framework canonical positions per heavy or light chain variable region, per heavy or light chain, and per antigen binding protein, and therefore any combination may be present in the antigen binding protein of the invention, provided that the canonical structure of the framework is maintained.

For example, the heavy chain variable framework may comprise V at position 2, L at position 4, V at position 20, C at position 22, A at position 24, G at position 26, F at position 29, W at position 36, W at position 47, M at position 48, M at position 69, A at position 78, M at position 80, Y at position 90, C at position 92, and R at position 94. For example, the light chain variable framework may comprise I at position 2, Q at position 3, M at position 4, C at position 23, W at position 35, F at position 36, S at position 46, Y at position 49, Y at position 71, C at position 88 and F at position 98.

One or more of the CDRs, corresponding CDRs, variant CDRs or binding units described herein may be present in the context of a human framework, for example as a humanised or chimeric variable domain.

The humanised heavy chain variable domain may comprise the CDRs listed in SEQ ID NO: 1-3; variant CDRs listed in SEQ ID NO: 82-97 and 110, and SEQ ID NO 109; corresponding CDRs; binding units; or variants thereof, within an acceptor antibody framework having 75% or greater, 80% or greater, 85% or greater, 90% or greater, 95% or greater, 98% or greater, 99% or greater or 100% identity in the framework regions to the human acceptor variable domain sequence in SEQ ID NO: 10. The humanised light chain variable domain may comprise the CDRs listed in SEQ ID NO: 4-6; variant CDRs listed in SEQ ID NO: 82-97 and 110, and SEQ ID NO 109; corresponding CDRs; binding units; or variants thereof, within an acceptor antibody framework having 75% or greater, 80% or greater, 85% or greater, 90% or greater, 95% or greater, 98% or greater, 99% or greater or 100% identity in the framework regions to the human acceptor variable domain sequence in SEQ ID NO: 11. In both SEQ ID NO: 10 and SEQ ID NO: 11 the position of CDRH3 has been denoted by X. The 10 X residues in SEQ ID NO: 10 and SEQ ID NO: 11, are a placeholder for the location of the CDR, and not a measure of the number of amino acid sequences in each CDR.

The invention also provides an antigen binding protein which binds to myostatin and comprises a heavy chain variable region selected from SEQ ID NO: 7 or 25. The antigen binding protein may comprise a light chain variable region selected from SEQ ID NO: 8 or 21.

The invention also provides an antigen binding protein which binds to myostatin and comprises any one of the following heavy chain and light chain variable region combinations: 10B3 (SEQ ID NO: 7 and SEQ ID NO: 8), 10B3C (SEQ ID NO: 25 and SEQ ID NO: 8), or 10B3C-C91S (SEQ ID NO: 25 and SEQ ID NO: 21). The antigen binding protein may also neutralise myostatin.

The invention also provides an antigen binding protein which binds to myostatin and comprises a heavy chain variable region selected from any one of SEQ ID NO: 12, 13, 14, 22 and 23. The antigen binding protein may comprise a light chain variable region selected from any one of SEQ ID NO: 15, 16, 17, 18 or 24. Any of the heavy chain variable regions may be combined with any of the light chain variable regions. The antigen binding protein may also neutralise myostatin.

The antigen binding protein may comprise any one of the following heavy chain and light chain variable region combinations: H0L0 (SEQ ID NO: 12 and SEQ ID NO: 15), H0L1 (SEQ ID NO: 12 and SEQ ID NO: 16), H0L2 (SEQ ID NO: 12 and SEQ ID NO: 17), H0L3 (SEQ ID NO: 12 and SEQ ID NO: 18), H1L0 (SEQ ID NO: 13 and SEQ ID NO: 15), H1L1 (SEQ ID NO: 13 and SEQ ID NO: 16), H1L2 (SEQ ID NO: 13 and SEQ ID NO: 17), H1L3 (SEQ ID NO: 13 and SEQ ID NO: 18), H2L0 (SEQ ID NO: 14 and SEQ ID NO: 15), H2L1 (SEQ ID NO: 14 and SEQ ID NO: 16), H2L2 (SEQ ID NO: 14 and SEQ ID NO: 17), H2L3 (SEQ ID NO: 14 and SEQ ID NO: 18), H2L2-C91S (SEQ ID NO: 14 and SEQ ID NO: 24).

The antibody heavy chain variable region may have 75% or greater, 80% or greater, 85% or greater, 90% or greater, 95% or greater, 98% or greater, 99% or greater or 100% identity to any one of SEQ ID NO: 7, 25, 12, 13, 14, 19, 20, 22 or 23. The antibody light chain variable region may have 75% or greater, 80% or greater, 85% or greater, 90% or greater, 95% or greater, 98% or greater, 99% or greater, or 100% identity to any one of SEQ ID NO: 8, 15, 16, 17, 18, 21 or 24.

The percentage identity of the variants of SEQ ID NO: 7, 25, 12, 13, 14, 19, 20, 22, 23, 8, 15, 16, 17, 18, 21 or 24 may be determined across the full length of the sequence.

The antibody heavy chain variable region may be a variant of any one of SEQ ID NO: 7, 25, 12, 13, 14, 19, 20, 22 or 23 which contains 30, 25, 20, 15, 10, 9, 8, 7, 6, 5, 4, 3, 2 or 1 amino acid substitutions, insertions or deletions. The antibody light chain variable region may be a variant of any one of SEQ ID NO: 8, 15, 16, 17, 18, 21 or 24 which contains 30, 25, 20, 15, 10, 9, 8, 7, 6, 5, 4, 3, 2 or 1 amino acid substitutions, insertions or deletions.

For example, the canonical CDRs and canonical framework residue substitutions described above may also be present in the variant heavy or light chain variable regions as variant sequences that are at least 75% identical or which contain up to 30 amino acid substitutions.

The substitution may comprise any one of the following: Y96L, G99D, G99S, G100A_K, P100B_F, P100B_I, W100E_F, F100G_N, F100G_S, F100G_Y, V102N, and V102S; in any one of the antibody heavy chain variable regions described above. In addition to any one of the substitutions described, the antibody heavy chain variable region may also comprise any one of the following substitutions: G55D, G55L, G55S, G55T or G55V, in any one of the antibody heavy chain variable regions described above.

The antibody heavy chain variable region may have the sequence of SEQ ID NO: 14 with the substitution F100G_Y. In addition to the substitution F100G_Y, any one of the following substitutions G55D, G55L, G55S, G55T or G55V may also be present. In particular, the antibody heavy chain variable region may have the sequence of SEQ ID NO: 14 with the following substitution: F100G_Y; or F100G_Y and G55S. The antibody heavy chain variable region may be paired with the light chain variable region of the sequence of SEQ ID NO: 24.

Any of the heavy chain variable regions may be combined with a suitable human constant region. Any of the light chain variable regions may be combined with a suitable constant region.

The invention also provides an antigen binding protein which binds to myostatin and comprises any one of the following heavy chain and light chain combinations: 10B3C (SEQ ID NO: 26 and SEQ ID NO: 27), or 10B3C-C91S (SEQ ID NO: 26 and SEQ ID NO: 37). The antigen binding protein may also neutralise myostatin.

The invention also provides an antigen binding protein which binds to myostatin and comprises a heavy chain selected from any one of SEQ ID NO: 28, 29, 30, 35, 36, 38, 39, 98 or 99. The antigen binding protein may comprise a light chain selected from any one of SEQ ID NO: 31, 32, 33, 34 or 40. Any of the heavy chains may be combined with any of the light chains. The antigen binding protein may also neutralise myostatin.

The antigen binding protein may comprise any one of the following heavy chain and light chain combinations: H0L0 (SEQ ID NO: 28 and SEQ ID NO: 31), H0L1 (SEQ ID NO: 28 and SEQ ID NO: 32), H0L2 (SEQ ID NO: 28 and SEQ ID NO: 33), H0L3 (SEQ ID NO: 28 and SEQ ID NO: 34), H1L0 (SEQ ID NO: 29 and SEQ ID NO: 31), H1L1 (SEQ ID NO: 29 and SEQ ID NO: 32), H1L2 (SEQ ID NO: 29 and SEQ ID NO: 33), H1L3 (SEQ ID NO: 29 and SEQ ID NO: 34), H2L0 (SEQ ID NO: 30 and SEQ ID NO: 31), H2L1 (SEQ ID NO: 30 and SEQ ID NO: 32), H2L2 (SEQ ID NO: 30 and SEQ ID NO: 33), H2L3 (SEQ ID NO: 30 and SEQ ID NO: 34), H2L2-C91S (SEQ ID NO: 30 and SEQ ID NO: 40), H2L2-C91S_F100G_Y Fc disabled (SEQ ID NO: 98 and SEQ ID NO: 40), or H2L2-C91S_G55S-F 100G_Y Fc disabled (SEQ ID NO: 99 and SEQ ID NO: 40).

The antibody heavy chain may have 75% or greater, 80% or greater, 85% or greater, 90% or greater, 95% or greater, 98% or greater, 99% or greater or 100% identity to any one of SEQ ID NO: 26, 28, 29, 30, 35, 36, 38, 39, 98 or 99. The antibody light chain may have 75% or greater, 80% or greater, 85% or greater, 90% or greater, 95% or greater, 98% or greater, 99% or greater, or 100% identity to any one of SEQ ID NO: 27, 31, 32, 33, 34, 37 or 40.

The percentage identity of the variants of SEQ ID NO: 26, 28, 29, 30, 35, 36, 38, 39, 98, 99, 27, 31, 32, 33, 34, 37 or 40 may be determined across the length of the sequence.

The antibody heavy chain may be a variant of any one of SEQ ID NO: 26, 28, 29, 30, 35, 36, 38, 39, 98 or 99 which contains 30, 25, 20, 15, 10, 9, 8, 7, 6, 5, 4, 3, 2 or 1 amino acid substitutions, insertions or deletions. The antibody light chain may be a variant of any one of SEQ ID NO: 27, 31, 32, 33, 34, 37 or 40 which contains 30, 25, 20, 15, 10, 9, 8, 7, 6, 5, 4, 3, 2 or 1 amino acid substitutions, insertions or deletions.

For example, the canonical CDRs and canonical framework residue substitutions described above may also be present in the variant heavy or light chains as variant sequences that are at least 75% identical or which contain up to 30 amino acid substitutions.

The substitution may comprise any one of the following: Y96L, G99D, G99S, G100A_K, P100B_F, P100B_I, W100E_F, F100G_S, F100G_N, F100G_Y, V102N, and V102S; in any one of the antibody heavy chains described above. In addition to any one of the substitutions described, the antibody heavy chain may also comprise any one of the following substitutions: G55D, G55L, G55S, G55T or G55V, in any one of the antibody heavy chains described above.

The antibody heavy chain may have the sequence of SEQ ID NO: 30 with the substitution F100G_Y. In addition to the substitution F100G_Y, any one of the following substitutions G55D, G55L, G55S, G55T or G55V may also be present. In particular, the antibody heavy chain may have the sequence of SEQ ID NO: 30 with the following substitution: F100G_Y; or F100G_Y and G55S. The antibody heavy chain may be paired with the light chain of the sequence of SEQ ID NO: 40.

Antigen binding proteins as described above, for example variants with a partial alteration of the sequence by chemical modification and/or insertion, deletion or substitution of one or more amino acid residues, or those with 75% or greater, 80% or greater, 85% or greater, 90% or greater, 95% or greater, 98% or greater, or 99% or greater identity to any of the sequences described above, may display a potency for binding to myostatin, as demonstrated by EC50, of within 10 fold, or within 5 fold of the potency demonstrated by 10B3 or 10B3 chimera (heavy chain: SEQ ID NO: 7 or 25, light chain: SEQ ID NO: 8). Potency for binding to myostatin, as demonstrated by EC50, may be carried out by an ELISA assay.

The antigen binding proteins of the invention may be Fc disabled. One way to achieve Fc disablement comprises the substitutions of alanine residues at positions 235 and 237 (EU index numbering) of the heavy chain constant region. For example, the antigen binding protein may be Fc disabled and comprise the sequence of SEQ ID NO: 98 (humanised heavy chain: H2_F100G_Y Fc disabled); or SEQ ID NO: 99 (humanised heavy chain: H2_G55S-F100G_Y Fc disabled). Alternatively, the antigen binding protein may be Fc enabled and not comprise the alanine substitutions at positions 235 and 237.

The antigen binding protein may bind to myostatin and compete for binding to myostatin with a reference antibody comprising a heavy chain variable region sequence of SEQ ID NO: 7 or 25, and a light chain variable region sequence of SEQ ID NO: 8; wherein the antigen binding protein does not bind to a peptide fragment of myostatin. The peptide fragment of myostatin may consist of SEQ ID NO: 81 (CCTPTKMSPINMLY). The peptide fragment of myostatin may be any fragment consisting of up to 14 amino acids of the myostatin sequence. The peptide fragment of myostatin may be linear. The peptide fragment of myostatin may be any fragment of the myostatin sequence, including the full length sequence, wherein the peptide fragment is linear. This may be assessed using the method described in Example 2.4 using an SRU BIND reader and biotinylated peptides captured onto a streptavidin coated biosensor plate.

Alternatively, the antigen binding protein may bind to myostatin and compete for binding to myostatin with a reference antibody comprising a heavy chain variable region sequence of SEQ ID NO: 7 or 25, and a light chain variable region sequence of SEQ ID NO: 8; wherein the antigen binding protein does not bind to an artificial peptide sequence consisting of SEQ ID NO: 74 (artificial myostatin linear peptide 37—SGSGCCTPTKMSPINMLY). The artificial peptide sequence may consist of any one of the sequences described in Table 7. The artificial peptide sequence may be linear. This may be assessed using the method described in Example 2.4 using an SRU BIND reader and biotinylated peptides captured onto a streptavidin coated biosensor plate.

The reference antibody may comprise the following heavy chain and light chain combination: 10B3C (SEQ ID NO: 26 and SEQ ID NO: 27). The heavy chain sequence SEQ ID NO: 26 comprises the variable domain sequence SEQ ID NO: 25; and the light chain sequence SEQ ID NO: 27 comprises the variable domain sequence SEQ ID NO: 8.

Competition between the antigen binding protein and the reference antibody may be determined by competition ELISA. Competition for neutralisation of myostatin may be determined by any one or a combination of: competition for binding to myostatin, for example as determined by ELISA, FMAT or BIAcore; competition for inhibition of myostatin binding to the ActRIIb receptor; and competition for inhibition of cell signalling resulting in luciferase expression in an A204 assay. A competing antigen binding protein may bind to the same epitope, an overlapping epitope, or an epitope in close proximity of the epitope to which the reference antibody binds.

The antigen binding protein may not bind significantly to the myostatin peptide fragment or artificial peptide sequence. The antigen binding protein may not bind to the myostatin peptide fragment or artificial peptide sequence at a ratio range of 1:1 to 1:10, of antigen binding protein to peptide, respectively.

Binding or lack of binding between the antigen binding protein and the myostatin peptide fragment or artificial peptide sequence may be determined by ELISA or by SDS PAGE using reducing conditions. For example, binding or lack of binding of the antigen binding protein to the linear full length myostatin sequence may be determined by reducing SDS PAGE.

The antigen binding proteins described herein may not bind to a peptide fragment of myostatin. The peptide fragment of myostatin may consist of SEQ ID NO: 81 (CCTPTKMSPINMLY). The peptide fragment of myostatin may be any fragment consisting of up to 14 amino acids of the myostatin sequence. The peptide fragment of myostatin may be linear. The peptide fragment of myostatin may be any fragment of the myostatin sequence, including the full length sequence, wherein the sequence is linear. This may be assessed using the method described in Example 2.4 using an SRU BIND reader and biotinylated peptides captured onto a streptavidin coated biosensor plate.

Alternatively, the antigen binding proteins described herein may not bind to an artificial peptide sequence consisting of SEQ ID NO: 74 (artificial myostatin linear peptide 37—SGSGCCTPTKMSPINMLY). The artificial peptide sequence may consist of any one of the sequences described in Table 7. The artificial peptide sequence may be linear. This may be assessed using the method described in Example 2.4 using an SRU BIND reader and biotinylated peptides captured onto a streptavidin coated biosensor plate.

The antigen binding protein may not bind significantly to the myostatin peptide fragment or artificial peptide sequence. The antigen binding protein may not bind to the myostatin peptide fragment or artificial peptide sequence at a ratio range of 1:1 to 1:10, respectively.

Binding or lack of binding between the antigen binding protein and the myostatin peptide fragment or artificial peptide sequence may be determined by ELISA or by SDS PAGE using reducing conditions. For example, binding or lack of binding of the antigen binding protein to the linear full length myostatin sequence may be determined by reducing (i.e. denaturing) SDS PAGE. For example, the method described in Example 2.4 using an SRU BIND reader and biotinylated peptides captured onto a streptavidin coated biosensor plate may be employed. The data in Example 2.4 suggest that 10B3 may bind a conformational sequence which may prove beneficial in the binding and neutralisation of native myostatin in vivo for therapeutic treatment.

The epitope of myostatin to which the antigen binding proteins described herein bind may be a conformational or discontinuous epitope. The antigen binding proteins described herein may not bind to a linear epitope on myostatin, for example the antigen binding protein may not bind to a reduced or denatured sample of myostatin. The conformational or discontinuous epitope may be identical to, similar to, or overlap with the myostatin receptor binding site. The epitope may be accessible when myostatin is in its mature form and as part of a dimer with another myostatin molecule (homodimer). The epitope may also be accessible when myostatin is in its mature form and as part of a tetramer with other myostatin binding molecules as described. The epitope may be distributed across two myostatin polypeptides. This type of discontinuous epitope may comprise sequences from each myostatin molecule. The sequences may, in the context of the dimer's tertiary and quaternary structure, be near enough to each other to form an epitope and be bound by an antigen binding protein. Conformational and/or discontinuous epitopes may be identified by known methods for example CLIPS™ (Pepscan Systems).

Subsequent analysis of the myostatin binding site of 10B3C using Pepscan, Chemically Linked Immunogenic Peptides on Scaffolds (CLIPS) technology, suggest that the “PRGSAGPCCTPTKMS” amino acid sequence of myostatin may be the binding site for the chimeric antibody. The Pepscan methodology uses constrained peptides.

The antigen binding protein may have a half life of at least 6 hours, at least 1 day, at least 2 days, at least 3 days, at least 4 days, at least 5 days, at least 7 days, or at least 9 days in vivo in humans, or in a murine animal model.

The myostatin polypeptide to which the antigen binding protein binds may be a recombinant polypeptide. Myostatin may be in solution or may be attached to a solid surface. For example, myostatin may be attached to beads such as magnetic beads. Myostatin may be biotinylated. The biotin molecule conjugated to myostatin may be used to immobilize myostatin on a solid surface by coupling biotinstreptavidin on the solid surface.

The antigen binding protein may be derived from rat, mouse, primate (e.g. cynomolgus, Old World monkey or Great Ape) or human. The antigen binding protein may be a humanised or chimeric antibody.

The antigen binding protein may comprise a constant region, which may be of any isotype or subclass. The constant region may be of the IgG isotype, for example IgG1, IgG2, IgG3, IgG4 or variants thereof. The antigen binding protein constant region may be IgG1.

Mutational changes to the Fc effector portion of the antibody can be used to change the affinity of the interaction between the FcRn and antibody to modulate antibody turnover. The half life of the antibody can be extended in vivo. This would be beneficial to patient populations as maximal dose amounts and maximal dosing frequencies could be achieved as a result of maintaining in vivo IC50 for longer periods of time. The Fc effector function of the antibody may be removed, in its entirety or in part, since myostatin is a soluble target. This removal may result in an increased safety profile.

The antigen binding protein comprising a constant region may have reduced ADCC and/or complement activation or effector functionality. The constant domain may comprise a naturally disabled constant region of IgG2 or IgG4 isotype or a mutated IgG1 constant domain. Examples of suitable modifications are described in EP0307434. One way to achieve Fc disablement comprises the substitutions of alanine residues at positions 235 and 237 (EU index numbering) of the heavy chain constant region.

The antigen binding protein may comprise one or more modifications selected from a mutated constant domain such that the antibody has enhanced effector functions/ADCC and/or complement activation. Examples of suitable modifications are described in Shields et al. J. Biol. Chem. (2001) 276:6591-6604, Lazar et al. PNAS (2006) 103:4005-4010 and U.S. Pat. No. 6,737,056, WO2004063351 and WO2004029207.

The antigen binding protein may comprise a constant domain with an altered glycosylation profile such that the antigen binding protein has enhanced effector functions/ADCC and/or complement activation. Examples of suitable methodologies to produce an antigen binding protein with an altered glycosylation profile are described in WO2003/011878, WO2006/014679 and EP1229125.

The present invention also provides a nucleic acid molecule which encodes an antigen binding protein as described herein. The nucleic acid molecule may comprise a sequence encoding (i) one or more CDRHs, the heavy chain variable sequence, or the full length heavy chain sequence; and (ii) one or more CDRLs, the light chain variable sequence, or the full length light chain sequence, with (i) and (ii) on the same nucleic acid molecule. Alternatively, the nucleic acid molecule which encodes an antigen binding protein described herein may comprise sequences encoding (a) one or more CDRHs, the heavy chain variable sequence, or the full length heavy chain sequence; or (b) one or more CDRLs, the light chain variable sequence, or the full length light chain sequence, with (a) and (b) on separate nucleic acid molecules.

The nucleic acid molecule which encodes the heavy chain may comprise SEQ ID NO: 41. The nucleic acid molecule which encodes the light chain may comprise SEQ ID NO: 42 or SEQ ID NO: 52.

The nucleic acid molecule which encodes the heavy chain may comprise any one of SEQ ID NO: 43, 44 or 45. The nucleic acid molecule which encodes the light chain may comprise any one of SEQ ID NO: 46, 47, 48, 49 or 55. The nucleic acid molecule(s) which encodes the antigen binding protein may comprise any one of the following heavy chain and light chain combinations: H0L0 (SEQ ID NO: 43 and SEQ ID NO: 46), H0L1 (SEQ ID NO: 43 and SEQ ID NO: 47), H0L2 (SEQ ID NO: 43 and SEQ ID NO: 48), H0L3 (SEQ ID NO: 43 and SEQ ID NO: 49), H1L0 (SEQ ID NO: 44 and SEQ ID NO: 46), H1L1 (SEQ ID NO: 44 and SEQ ID NO: 47), H1L2 (SEQ ID NO: 44 and SEQ ID NO: 48), H1L3 (SEQ ID NO: 44 and SEQ ID NO: 49), H2L0 (SEQ ID NO: 45 and SEQ ID NO: 46), H2L1 (SEQ ID NO: 45 and SEQ ID NO: 47), H2L2 (SEQ ID NO: 45 and SEQ ID NO: 48), H2L3 (SEQ ID NO: 45 and SEQ ID NO: 49), H2L2-C91S (SEQ ID NO: 45 and SEQ ID NO: 55).

The nucleic acid molecules described above may also encode a heavy chain with any one of the following substitutions: Y96L, G99D, G99S, G100A_K, P100B_F, P100B_I, W100E_F, F100G_N, F100G_Y, F100G_S, V102N, and V102S. In addition to, or as an alternative to, any one of the substitutions described, the nucleic acid molecules may also encode heavy chains comprising any one of the following substitutions: G55D, G55L, G55S, G55T or G55V. The nucleic acid molecules described above may also encode a light chain with the following substitution: C91S.

The nucleic acid molecule may have the sequence of SEQ ID NO: 45 with a substitution that encodes F100G_Y. In addition to the substitution F100G_Y, any one of the following substitutions G55D, G55L, G55S, G55T or G55V may also be present. In particular, the nucleic acid molecule may have the sequence of SEQ ID NO: 45 with a substitution that encodes: F100G_Y, or F100G_Y and G55S. The nucleic acid molecule that encodes the heavy chain may be paired with a nucleic acid molecule of the sequence of SEQ ID NO: 55 that encodes the light chain.

The present invention also provides an expression vector comprising a nucleic acid molecule as described herein. Also provided is a recombinant host cell comprising an expression vector as described herein.

The antigen binding protein described herein may be produced in a suitable host cell. A method for the production of the antigen binding protein as described herein may comprise the step of culturing a host cell as described herein and recovering the antigen binding protein. A recombinant transformed, transfected, or transduced host cell may comprise at least one expression cassette, whereby said expression cassette comprises a polynucleotide encoding a heavy chain of the antigen binding protein described herein and further comprises a polynucleotide encoding a light chain of the antigen binding protein described herein. Alternatively, a recombinant transformed, transfected or transduced host cell may comprise at least one expression cassette, whereby a first expression cassette comprises a polynucleotide encoding a heavy chain of the antigen binding protein described herein and further comprise a second cassette comprising a polynucleotide encoding a light chain of the antigen binding protein described herein. A stably transformed host cell may comprise a vector comprising one or more expression cassettes encoding a heavy chain and/or a light chain of the antigen binding protein described herein. For example such host cells may comprise a first vector encoding the light chain and a second vector encoding the heavy chain.

The host cell may be eukaryotic, for example mammalian. Examples of such cell lines include CHO or NS0. The host cell may be a non-human host cell. The host cell may be a non-embryonic host cell. The host cell may be cultured in a culture media, for example serum-free culture media. The antigen binding protein may be secreted by the host cell into the culture media. The antigen binding protein can be purified to at least 95% or greater (e.g. 98% or greater) with respect to said culture media containing the antigen binding protein.

A pharmaceutical composition comprising the antigen binding protein and a pharmaceutically acceptable carrier may be provided. A kit-of-parts comprising the pharmaceutical composition together with instructions for use may be provided. For convenience, the kit may comprise the reagents in predetermined amounts with instructions for use.

### Antibody Structures

### Intact Antibodies

The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b.

The more conserved portions of the variable region are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies.

The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1q component of the complement cascade.

The human IgG2 constant region has been reported to essentially lack the ability to activate complement by the classical pathway or to mediate antibody-dependent cellular cytotoxicity. The IgG4 constant region has been reported to lack the ability to activate complement by the classical pathway and mediates antibody-dependent cellular cytotoxicity only weakly. Antibodies essentially lacking these effector functions may be termed ‘non-lytic’ antibodies.

### Human Antibodies

Human antibodies may be produced by a number of methods known to those of skill in the art. Human antibodies can be made by the hybridoma method using human myeloma or mouse-human heteromyeloma cells lines see Kozbor (1984) J. Immunol. 133, 3001, and Brodeur, Monoclonal Antibody Production Techniques and Applications, 51-63 (Marcel Dekker Inc, 1987). Alternative methods include the use of phage libraries or transgenic mice both of which utilize human variable region repertories (see Winter (1994) Annu Rev. Immunol 12: 433-455; Green (1999) J. Immunol. Methods 231: 11-23).

Several strains of transgenic mice are now available wherein their mouse immunoglobulin loci has been replaced with human immunoglobulin gene segments (see Tomizuka (2000) PNAS 97: 722-727; Fishwild (1996) Nature Biotechnol. 14: 845-851; Mendez (1997) Nature Genetics, 15: 146-156). Upon antigen challenge such mice are capable of producing a repertoire of human antibodies from which antibodies of interest can be selected.

Phage display technology can be used to produce human antigen binding proteins (and fragments thereof), see McCafferty (1990) Nature 348: 552-553 and Griffiths et al. (1994) EMBO 13: 3245-3260.

The technique of affinity maturation (Marks Bio/technol (1992) 10: 779-783) may be used to improve binding affinity wherein the affinity of the primary human antibody is improved by sequentially replacing the H and L chain variable regions with naturally occurring variants and selecting on the basis of improved binding affinities. Variants of this technique such as “epitope imprinting” are now also available, see for example WO 93/06213; Waterhouse (1993) Nucl. Acids Res. 21: 2265-2266.

### Chimeric and Humanised Antibodies

Chimeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chains of the antibody. Hybridoma cells serve as a typical source of such DNA. Once isolated, the DNA is placed into expression vectors which are then transfected into host cells such as E. coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions, see for example Morrison (1984) PNAS 81: 6851.

A large decrease in immunogenicity can be achieved by grafting only the CDRs of a non-human (e.g. murine) antibodies (“donor” antibodies) onto human framework (“acceptor framework”) and constant regions to generate humanised antibodies (see Jones et al. (1986) Nature 321: 522-525; and Verhoeyen et al. (1988) Science 239: 1534-1536). However, CDR grafting per se may not result in the complete retention of antigen-binding properties and it is frequently found that some framework residues (sometimes referred to as “back mutations”) of the donor antibody need to be preserved in the humanised molecule if significant antigen-binding affinity is to be recovered (see Queen et al. (1989) PNAS 86: 10,029-10,033: Co et al. (1991) Nature 351: 501-502). In this case, human variable regions showing the greatest sequence homology to the non-human donor antibody are chosen from a database in order to provide the human framework (FR). The selection of human FRs can be made either from human consensus or individual human antibodies. Where necessary, key residues from the donor antibody can be substituted into the human acceptor framework to preserve CDR conformations. Computer modelling of the antibody maybe used to help identify such structurally important residues, see WO 99/48523.

Alternatively, humanisation maybe achieved by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan et al. (1991) Mol. Immunol. 28: 489-498; and Pedersen et al. (1994) J. Mol. Biol. 235: 959-973). Therefore it is possible to reduce the immunogenicity of a non-human Fv by replacing exposed residues in its framework regions that differ from those usually found in human antibodies. Because protein antigenicity may be correlated with surface accessibility, replacement of the surface residues may be sufficient to render the mouse variable region “invisible” to the human immune system (see also Mark et al. (1994) in Handbook of Experimental Pharmacology Vol. 113: The pharmacology of Monoclonal Antibodies, Springer-Verlag, 105-134). This procedure of humanisation is referred to as “veneering” because only the surface of the antibody is altered, the supporting residues remain undisturbed. Further alternative approaches include that set out in WO04/006955 and the procedure of Humaneering™ (Kalobios) which makes use of bacterial expression systems and produces antibodies that are close to human germline in sequence (Alfenito-M Advancing Protein Therapeutics January 2007, San Diego, Calif.).

### Bispecific Antigen Binding Proteins

A bispecific antigen binding protein is an antigen binding protein having binding specificities for at least two different epitopes. Methods of making such antigen binding proteins are known in the art. Traditionally, the recombinant production of bispecific antigen binding proteins is based on the co-expression of two immunoglobulin H chain-L chain pairs, where the two H chains have different binding specificities, see Millstein et al. (1983) Nature 305: 537-539; WO 93/08829; and Traunecker et al. (1991) EMBO 10: 3655-3659. Because of the random assortment of H and L chains, a potential mixture of ten different antibody structures are produced of which only one has the desired binding specificity. An alternative approach involves fusing the variable domains with the desired binding specificities to heavy chain constant region comprising at least part of the hinge region, CH2 and CH3 regions. The CH1 region containing the site necessary for light chain binding may be present in at least one of the fusions. DNA encoding these fusions, and if desired the L chain are inserted into separate expression vectors and are then co-transfected into a suitable host organism. It is possible though to insert the coding sequences for two or all three chains into one expression vector. In one approach, the bispecific antibody is composed of a H chain with a first binding specificity in one arm and a H-L chain pair, providing a second binding specificity in the other arm, see WO 94/04690. Also see Suresh et al. (1986) Methods in Enzymology 121: 210.

### Antigen Binding Fragments

Fragments lacking the constant region lack the ability to activate complement by the classical pathway or to mediate antibody-dependent cellular cytotoxicity. Traditionally such fragments are produced by the proteolytic digestion of intact antibodies by e.g. papain digestion (see for example, WO 94/29348) but may be produced directly from recombinantly transformed host cells. For the production of ScFv, see Bird et al. (1988) Science 242: 423-426. In addition, antigen binding fragments may be produced using a variety of engineering techniques as described below.

Fv fragments appear to have lower interaction energy of their two chains than Fab fragments. To stabilise the association of the V_{H} and V_{L} domains, they have been linked with peptides (Bird et al. (1988) Science 242: 423-426; Huston et al. (1988) PNAS 85(16): 5879-5883), disulphide bridges (Glockshuber et al. (1990) Biochemistry 29: 1362-1367) and “knob in hole” mutations (Zhu et al. (1997) Protein Sci., 6: 781-788). ScFv fragments can be produced by methods well known to those skilled in the art, see Whitlow et al. (1991) Methods Companion Methods Enzymol, 2: 97-105 and Huston et al. (1993) Int. Rev. Immunol 10: 195-217. ScFv may be produced in bacterial cells such as E. coli or in eukaryotic cells. One disadvantage of ScFv is the monovalency of the product, which precludes an increased avidity due to polyvalent binding, and their short half-life. Attempts to overcome these problems include bivalent (ScFv′)₂ produced from ScFv containing an additional C-terminal cysteine by chemical coupling (Adams et al. (1993) Can. Res 53: 4026-4034; and McCartney et al. (1995) Protein Eng. 8: 301-314) or by spontaneous site-specific dimerisation of ScFv containing an unpaired C-terminal cysteine residue (see Kipriyanov et al. (1995) Cell. Biophys 26: 187-204). Alternatively, ScFv can be forced to form multimers by shortening the peptide linker to 3 to 12 residues to form “diabodies”, see Holliger et al. (1993) PNAS 90: 6444-6448. Reducing the linker still further can result in ScFv trimers (“triabodies”, see Kortt et al. (1997) Protein Eng 10: 423-433) and tetramers (“tetrabodies”, see Le Gall et al. (1999) FEBS Lett, 453: 164-168). Construction of bivalent ScFv molecules can also be achieved by genetic fusion with protein dimerising motifs to form “miniantibodies” (see Pack et al. (1992) Biochemistry 31: 1579-1584) and “minibodies” (see Hu et al. (1996) Cancer Res. 56: 3055-3061). ScFv-Sc-Fv tandems ((ScFv)₂) may also be produced by linking two ScFv units by a third peptide linker, see Kurucz et al. (1995) J. Immol. 154: 4576-4582. Bispecific diabodies can be produced through the noncovalent association of two single chain fusion products consisting of V_{H} domain from one antibody connected by a short linker to the V_{L} domain of another antibody, see Kipriyanov et al. (1998) Int. J. Can 77: 763-772. The stability of such bispecific diabodies can be enhanced by the introduction of disulphide bridges or “knob in hole” mutations as described supra or by the formation of single chain diabodies (ScDb) wherein two hybrid ScFv fragments are connected through a peptide linker see Kontermann et al. (1999) J. Immunol. Methods 226:179-188. Tetravalent bispecific molecules are available by e.g. fusing a ScFv fragment to the CH3 domain of an IgG molecule or to a Fab fragment through the hinge region, see Coloma et al. (1997) Nature Biotechnol. 15: 159-163. Alternatively, tetravalent bispecific molecules have been created by the fusion of bispecific single chain diabodies (see Alt et al. (1999) FEBS Lett 454: 90-94. Smaller tetravalent bispecific molecules can also be formed by the dimerization of either ScFv-ScFv tandems with a linker containing a helix-loop-helix motif (DiBi miniantibodies, see Muller et al. (1998) FEBS Lett 432: 45-49) or a single chain molecule comprising four antibody variable domains (V_{H} and V_{L}) in an orientation preventing intramolecular pairing (tandem diabody, see Kipriyanov et al. (1999) J. Mol. Biol. 293: 41-56). Bispecific F(ab′)₂ fragments can be created by chemical coupling of Fab′ fragments or by heterodimerization through leucine zippers (see Shalaby et al. (1992) J. Exp. Med. 175: 217-225; and Kostelny et al. (1992), J. Immunol. 148: 1547-1553). Also available are isolated V_{H} and V_{L} domains (Domantis plc), see U.S. Pat. No. 6,248,516; U.S. Pat. No. 6,291,158; and U.S. Pat. No. 6,172,197.

### Heteroconjugate Antibodies

Heteroconjugate antibodies are composed of two covalently joined antibodies formed using any convenient cross-linking methods. See, for example, U.S. Pat. No. 4,676,980.

### Other Modifications

The antigen binding proteins of the present invention may comprise other modifications to enhance or change their effector functions. The interaction between the Fc region of an antibody and various Fc receptors (FcγR) is believed to mediate the effector functions of the antibody which include antibody-dependent cellular cytotoxicity (ADCC), fixation of complement, phagocytosis and half-life/clearance of the antibody. Various modifications to the Fc region of antibodies may be carried out depending on the desired property. For example, specific mutations in the Fc region to render an otherwise lytic antibody, non-lytic is detailed in EP 0629 240 and EP 0307 434 or one may incorporate a salvage receptor binding epitope into the antibody to increase serum half life see U.S. Pat. No. 5,739,277. Human Fcγ receptors include FcγR (I), FcγRIIa, FcγRIIb, FcγRIIIa and neonatal FcRn. Shields et al. (2001) J. Biol. Chem. 276: 6591-6604 demonstrated that a common set of IgG1 residues is involved in binding all FcγRs, while FcγRII and FcγRIII utilize distinct sites outside of this common set. One group of IgG1 residues reduced binding to all FcγRs when altered to alanine: Pro-238, Asp-265, Asp-270, Asn-297 and Pro-239. All are in the IgG CH2 domain and clustered near the hinge joining CH1 and CH2. While FcγRI utilizes only the common set of IgG1 residues for binding, FcγRII and FcγRIII interact with distinct residues in addition to the common set. Alteration of some residues reduced binding only to FcγRII (e.g. Arg-292) or FcγRIII (e.g. Glu-293). Some variants showed improved binding to FcγRII or FcγRIII but did not affect binding to the other receptor (e.g. Ser-267Ala improved binding to FcγRII but binding to FcγRIII was unaffected). Other variants exhibited improved binding to FcγRII or FcγRIII with reduction in binding to the other receptor (e.g. Ser-298Ala improved binding to FcγRIII and reduced binding to FcγRII). For FcγRIIIa, the best binding IgG1 variants had combined alanine substitutions at Ser-298, Glu-333 and Lys-334. The neonatal FcRn receptor is believed to be involved in both antibody clearance and the transcytosis across tissues (see Junghans (1997) Immunol. Res 16: 29-57; and Ghetie et al. (2000) Annu Rev. Immunol. 18: 739-766). Human IgG1 residues determined to interact directly with human FcRn includes Ile253, Ser254, Lys288, Thr307, Gln311, Asn434 and His435. Substitutions at any of the positions described in this section may enable increased serum half-life and/or altered effector properties of the antibodies.

Other modifications include glycosylation variants of the antibodies. Glycosylation of antibodies at conserved positions in their constant regions is known to have a profound effect on antibody function, particularly effector functioning such as those described above, see for example, Boyd et al. (1996) Mol. Immunol. 32: 1311-1318. Glycosylation variants of the antibodies or antigen binding fragments thereof wherein one or more carbohydrate moiety is added, substituted, deleted or modified are contemplated. Introduction of an asparagine-X-serine or asparagine-X-threonine motif creates a potential site for enzymatic attachment of carbohydrate moieties and may therefore be used to manipulate the glycosylation of an antibody. In Raju et al. (2001) Biochemistry 40: 8868-8876 the terminal sialyation of a TNFR-IgG immunoadhesin was increased through a process of regalactosylation and/or resialylation using beta-1,4-galactosyltransferace and/or alpha, 2,3 sialyltransferase. Increasing the terminal sialylation is believed to increase the half-life of the immunoglobulin. Antibodies, in common with most glycoproteins, are typically produced as a mixture of glycoforms. This mixture is particularly apparent when antibodies are produced in eukaryotic, particularly mammalian cells. A variety of methods have been developed to manufacture defined glycoforms, see Zhang et al. (2004) Science 303: 371: Sears et al. (2001) Science 291: 2344; Wacker et al. (2002) Science 298: 1790; Davis et al. (2002) Chem. Rev. 102: 579; Hang et al. (2001) Acc. Chem. Res 34: 727. The antibodies (for example of the IgG isotype, e.g. IgG1) as herein described may comprise a defined number (e.g. 7 or less, for example 5 or less, such as two or a single) of glycoform(s).

The antibodies may be coupled to a non-proteinaeous polymer such as polyethylene glycol (PEG), polypropylene glycol or polyoxyalkylene. Conjugation of proteins to PEG is an established technique for increasing half-life of proteins, as well as reducing antigenicity and immunogenicity of proteins. The use of PEGylation with different molecular weights and styles (linear or branched) has been investigated with intact antibodies as well as Fab′ fragments, see Koumenis et al. (2000) Int. J. Pharmaceut. 198: 83-95.

### Production Methods

Antigen binding proteins may be produced in transgenic organisms such as goats (see Pollock et al. (1999) J. Immunol. Methods 231: 147-157), chickens (see Morrow (2000) Genet. Eng. News 20:1-55, mice (see Pollock et al.) or plants (see Doran (2000) Curr. Opinion Biotechnol. 11: 199-204; Ma (1998) Nat. Med. 4: 601-606; Baez et al. (2000) BioPharm 13: 50-54; Stoger et al. (2000) Plant Mol. Biol. 42: 583-590).

Antigen binding proteins may also be produced by chemical synthesis. However, antigen binding proteins are typically produced using recombinant cell culturing technology well known to those skilled in the art. A polynucleotide encoding the antigen binding protein is isolated and inserted into a replicable vector such as a plasmid for further cloning (amplification) or expression. One expression system is a glutamate synthetase system (such as sold by Lonza Biologics), particularly where the host cell is CHO or NS0. Polynucleotide encoding the antigen binding protein is readily isolated and sequenced using conventional procedures (e.g. oligonucleotide probes). Vectors that may be used include plasmid, virus, phage, transposons, minichromosomes of which plasmids are typically used. Generally such vectors further include a signal sequence, origin of replication, one or more marker genes, an enhancer element, a promoter and transcription termination sequences operably linked to the antigen binding protein polynucleotide so as to facilitate expression. Polynucleotide encoding the light and heavy chains may be inserted into separate vectors and introduced (for example by transformation, transfection, electroporation or transduction) into the same host cell concurrently or sequentially or, if desired both the heavy chain and light chain can be inserted into the same vector prior to said introduction.

Codon optimisation may be used with the intent that the total level of protein produced by the host cell is greater when transfected with the codon-optimised gene in comparison with the level when transfected with the wild-type sequence. Several methods have been published (Nakamura et al. (1996) Nucleic Acids Research 24: 214-215; WO98/34640; WO97/11086). Due to the redundancy of the genetic code, alternative polynucleotides to those disclosed herein (particularly those codon optimised for expression in a given host cell) may also encode the antigen binding proteins described herein. The codon usage of the antigen binding protein of this invention thereof can be modified to accommodate codon bias of the host cell such to augment transcript and/or product yield (eg Hoekema et al Mol Cell Biol 1987 7(8): 2914-24). The choice of codons may be based upon suitable compatibility with the host cell used for expression.

### Signal Sequences

Antigen binding proteins may be produced as a fusion protein with a heterologous signal sequence having a specific cleavage site at the N-terminus of the mature protein. The signal sequence should be recognised and processed by the host cell. For prokaryotic host cells, the signal sequence may be for example an alkaline phosphatase, penicillinase, or heat stable enterotoxin II leaders. For yeast secretion the signal sequences may be for example a yeast invertase leader, a factor leader or acid phosphatase leaders see e.g. WO90/13646. In mammalian cell systems, viral secretory leaders such as herpes simplex gD signal and a native immunoglobulin signal sequence may be suitable. Typically the signal sequence is ligated in reading frame to DNA encoding the antigen binding protein. A signal sequence such as that shown in SEQ ID NO: 9 may be used.

### Origin of Replication

Origin of replications are well known in the art with pBR322 suitable for most gram-negative bacteria, 2μ plasmid for most yeast and various viral origins such as SV40, polyoma, adenovirus, VSV or BPV for most mammalian cells. Generally the origin of replication component is not needed for mammalian expression vectors but the SV40 may be used since it contains the early promoter.

### Selection Marker

Typical selection genes encode proteins that (a) confer resistance to antibiotics or other toxins e.g. ampicillin, neomycin, methotrexate or tetracycline or (b) complement auxiotrophic deficiencies or supply nutrients not available in the complex media or (c) combinations of both. The selection scheme may involve arresting growth of the host cell. Cells, which have been successfully transformed with the genes encoding the antigen binding protein, survive due to e.g. drug resistance conferred by the co-delivered selection marker. One example is the DHFR selection marker wherein transformants are cultured in the presence of methotrexate. Cells can be cultured in the presence of increasing amounts of methotrexate to amplify the copy number of the exogenous gene of interest. CHO cells are a particularly useful cell line for the DHFR selection. A further example is the glutamate synthetase expression system (Lonza Biologics). An example of a selection gene for use in yeast is the trp1 gene, see Stinchcomb et al. (1979) Nature 282: 38.

### Promoters

Suitable promoters for expressing antigen binding proteins are operably linked to DNA/polynucleotide encoding the antigen binding protein. Promoters for prokaryotic hosts include phoA promoter, beta-lactamase and lactose promoter systems, alkaline phosphatase, tryptophan and hybrid promoters such as Tac. Promoters suitable for expression in yeast cells include 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase. Inducible yeast promoters include alcohol dehydrogenase 2, isocytochrome C, acid phosphatase, metallothionein and enzymes responsible for nitrogen metabolism or maltose/galactose utilization.

Promoters for expression in mammalian cell systems include viral promoters such as polyoma, fowlpox and adenoviruses (e.g. adenovirus 2), bovine papilloma virus, avian sarcoma virus, cytomegalovirus (in particular the immediate early gene promoter), retrovirus, hepatitis B virus, actin, rous sarcoma virus (RSV) promoter and the early or late Simian virus 40. Of course the choice of promoter is based upon suitable compatibility with the host cell used for expression. A first plasmid may comprise a RSV and/or SV40 and/or CMV promoter, DNA encoding light chain variable region (V_{L}), κC region together with neomycin and ampicillin resistance selection markers and a second plasmid comprising a RSV or SV40 promoter, DNA encoding the heavy chain variable region (V_{H}), DNA encoding the γ1 constant region, DHFR and ampicillin resistance markers.

### Enhancer Element

Where appropriate, e.g. for expression in higher eukaryotes, an enhancer element operably linked to the promoter element in a vector may be used. Mammalian enhancer sequences include enhancer elements from globin, elastase, albumin, fetoprotein and insulin. Alternatively, one may use an enhancer element from a eukaroytic cell virus such as SV40 enhancer (at bp100-270), cytomegalovirus early promoter enhancer, polyma enhancer, baculoviral enhancer or murine IgG2a locus (see WO04/009823). The enhancer may be located on the vector at a site upstream to the promoter. Alternatively, the enhancer may be located elsewhere, for example within the untranslated region or downstream of the polyadenylation signal. The choice and positioning of enhancer may be based upon suitable compatibility with the host cell used for expression.

### Polyadenylation/Termination

In eukaryotic systems, polyadenylation signals are operably linked to DNA/polynucleotide encoding the antigen binding protein. Such signals are typically placed 3′ of the open reading frame. In mammalian systems, non-limiting examples include signals derived from growth hormones, elongation factor-1 alpha and viral (eg SV40) genes or retroviral long terminal repeats. In yeast systems non-limiting examples of polydenylation/termination signals include those derived from the phosphoglycerate kinase (PGK) and the alcohol dehydrogenase 1 (ADH) genes. In prokaryotic system polyadenylation signals are typically not required and it is instead usual to employ shorter and more defined terminator sequences. The choice of polyadenylation/termination sequences may be based upon suitable compatibility with the host cell used for expression.

### Other Methods/Elements for Enhanced Yields

In addition to the above, other features that can be employed to enhance yields include chromatin remodelling elements, introns and host-cell specific codon modification.

### Host Cells

Suitable host cells for cloning or expressing vectors encoding antigen binding proteins are prokaroytic, yeast or higher eukaryotic cells. Suitable prokaryotic cells include eubacteria e.g. enterobacteriaceae such as Escherichia e.g. E. coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. licheniformis (see DD 266 710), Pseudomonas such as P. aeruginosa and Streptomyces. Of the yeast host cells, Saccharomyces cerevisiae, Schizosaccharomyces pombe, Kluyveromyces (e.g. ATCC 16,045; 12,424; 24178; 56,500), yarrowia (EP402, 226), Pichia pastoris (EP 183 070, see also Peng et al. (2004) J. Biotechnol. 108: 185-192), Candida, Trichoderma reesia (EP 244 234), Penicillin, Tolypocladium and Aspergillus hosts such as A. nidulans and A. niger are also contemplated.

Higher eukaryotic host cells include mammalian cells such as COS-1 (ATCC No. CRL 1650) COS-7 (ATCC CRL 1651), human embryonic kidney line 293, baby hamster kidney cells (BHK) (ATCC CRL.1632), BHK570 (ATCC NO: CRL 10314), 293 (ATCC NO. CRL 1573), Chinese hamster ovary cells CHO (e.g. CHO-K1, ATCC NO: CCL 61, DHFR-CHO cell line such as DG44 (see Urlaub et al. (1986) Somatic Cell Mol. Genet. 12: 555-556), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NS0 (see U.S. Pat. No. 5,807,715), Sp2/0, Y0.

Such host cells may also be further engineered or adapted to modify quality, function and/or yield of the antigen binding protein. Non-limiting examples include expression of specific modifying (e.g. glycosylation) enzymes and protein folding chaperones.

### Cell Culturing Methods

Host cells transformed with vectors encoding antigen binding proteins may be cultured by any method known to those skilled in the art. Host cells may be cultured in spinner flasks, roller bottles or hollow fibre systems but for large scale production that stirred tank reactors are used particularly for suspension cultures. The stirred tankers may be adapted for aeration using e.g. spargers, baffles or low shear impellers. For bubble columns and airlift reactors direct aeration with air or oxygen bubbles maybe used. Where the host cells are cultured in a serum free culture media, the media is supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly invertebrate host cells may utilise a variety of operational modes such as fed-batch, repeated batch processing (see Drapeau et al. (1994) Cytotechnology 15: 103-109), extended batch process or perfusion culture. Although recombinantly transformed mammalian host cells may be cultured in serum-containing media such as fetal calf serum (FCS), for example such host cells are cultured in synthetic serum-free media such as disclosed in Keen et al. (1995) Cytotechnology 17: 153-163, or commercially available media such as ProCHO-CDM or UltraCHO™ (Cambrex NJ, USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg et al. (1995) in Animal Cell Technology: Developments towards the 21st century (Beuvery et al. eds, 619-623, Kluwer Academic publishers).

Antigen binding proteins secreted into the media may be recovered and purified using a variety of techniques to provide a degree of purification suitable for the intended use. For example the use of antigen binding proteins for the treatment of human patients typically mandates at least 95% purity, more typically 98% or 99% or greater purity (compared to the crude culture medium). Cell debris from the culture media is typically removed using centrifugation followed by a clarification step of the supernatant using e.g. microfiltration, ultrafiltration and/or depth filtration. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. The antibodies, following various clarification steps, can be captured using Protein A or G affinity chromatography. Further chromatography steps can follow such as ion exchange and/or HA chromatography, anion or cation exchange, size exclusion chromatography and ammonium sulphate precipitation. Various virus removal steps may also be employed (e.g. nanofiltration using e.g. a DV-20 filter). Following these various steps, a purified (for example a monoclonal) preparation comprising at least 75 mg/ml or greater, or 100 mg/ml or greater, of the antigen binding protein is provided. Such preparations are substantially free of aggregated forms of antigen binding proteins.

Bacterial systems may be used for the expression of antigen binding fragments. Such fragments can be localised intracellularly, within the periplasm or secreted extracellularly. Insoluble proteins can be extracted and refolded to form active proteins according to methods known to those skilled in the art, see Sanchez et al. (1999) J. Biotechnol. 72: 13-20; and Cupit et al. (1999) Lett Appl Microbiol 29: 273-277.

Deamidation is a chemical reaction in which an amide functional group is removed. In biochemistry, the reaction is important in the degradation of proteins because it damages the amide-containing side chains of the amino acids asparagine and glutamine. Deamidation reactions are believed to be one of the factors that can limit the useful lifetime of a protein, they are also one of the most common post-translational modifications occurring during the manufacture of therapeutic proteins. For example, a reduction or loss of in vitro or in vivo biological activity has been reported for recombinant human DNAse and recombinant soluble CD4, whereas other recombinant proteins appear to be unaffected. The ability of the antigen binding proteins described herein to bind to myostatin seems to be unaffected under stress conditions that induce deamidation. Thus, the biological activity of the antigen binding proteins described herein and their useful lifetime is unlikely to be affected by deamidation.

### Pharmaceutical Compositions

The terms diseases, disorders and conditions are used interchangeably. Purified preparations of an antigen binding protein as described herein may be incorporated into pharmaceutical compositions for use in the treatment of the human diseases described herein. The pharmaceutical composition can be used in the treatment of diseases where myostatin contributes to the disease or where neutralising the activity of myostatin will be beneficial. The pharmaceutical composition comprising a therapeutically effective amount of the antigen binding protein described herein can be used in the treatment of diseases responsive to neutralisation of myostatin.

The pharmaceutical preparation may comprise an antigen binding protein in combination with a pharmaceutically acceptable carrier. The antigen binding protein may be administered alone, or as part of a pharmaceutical composition.

Typically such compositions comprise a pharmaceutically acceptable carrier as known and called for by acceptable pharmaceutical practice, see e.g. Remingtons Pharmaceutical Sciences, 16th edition (1980) Mack Publishing Co. Examples of such carriers include sterilised carriers such as saline, Ringers solution or dextrose solution, optionally buffered with suitable buffers to a pH within a range of 5 to 8.

Pharmaceutical compositions may be administered by injection or continuous infusion (e.g. intravenous, intraperitoneal, intradermal, subcutaneous, intramuscular or intraportal). Such compositions are suitably free of visible particulate matter. Pharmaceutical compositions may comprise between 1 mg to 10 g of antigen binding protein, for example between 5 mg and 1 g of antigen binding protein. Alternatively, the composition may comprise between 5 mg and 500 mg, for example between 5 mg and 50 mg.

Methods for the preparation of such pharmaceutical compositions are well known to those skilled in the art. Pharmaceutical compositions may comprise between 1 mg to 10 g of antigen binding protein in unit dosage form, optionally together with instructions for use. Pharmaceutical compositions may be lyophilised (freeze dried) for reconstitution prior to administration according to methods well known or apparent to those skilled in the art. Where antibodies have an IgG1 isotype, a chelator of copper, such as citrate (e.g. sodium citrate) or EDTA or histidine, may be added to the pharmaceutical composition to reduce the degree of copper-mediated degradation of antibodies of this isotype, see EP0612251. Pharmaceutical compositions may also comprise a solubiliser such as arginine base, a detergent/anti-aggregation agent such as polysorbate 80, and an inert gas such as nitrogen to replace vial headspace oxygen.

Effective doses and treatment regimes for administering the antigen binding protein are generally determined empirically and may be dependent on factors such as the age, weight and health status of the patient and disease or disorder to be treated. Such factors are within the purview of the attending physician. Guidance in selecting appropriate doses may be found in e.g. Smith et al (1977) Antibodies in human diagnosis and therapy, Raven Press, New York. Thus the antigen binding protein of the invention may be administered at a therapeutically effective amount.

The dosage of antigen binding protein administered to a subject is generally between 1 μg/kg to 150 mg/kg, between 0.1 mg/kg and 100 mg/kg, between 0.5 mg/kg and 50 mg/kg, between 1 and 25 mg/kg or between 1 and 10 mg/kg of the subject's body weight. For example, the dose may be 10 mg/kg, 30 mg/kg, or 60 mg/kg. The antigen binding protein may be administered parenterally, for example subcutaneously, intravenously or intramuscularly.

If desired, the effective daily dose of a therapeutic composition may be administered as two, three, four, five, six or more sub-doses administered separately at appropriate intervals, optionally, in unit dosage forms. For example, the dose may be administered subcutaneously, once every 14 or 28 days in the form of multiple sub-doses on each day of administration.

The administration of a dose may be by intravenous infusion, typically over a period of from 15 minutes to 24 hours, such as of from 2 to 12 hours, or from 2 to 6 hours. This may result in reduced toxic side effects.

The administration of a dose may be repeated one or more times as necessary, for example, three times daily, once every day, once every 2 days, once a week, once a fortnight, once a month, once every 3 months, once every 6 months, or once every 12 months. The antigen binding proteins may be administered by maintenance therapy, for example once a week for a period of 6 months or more. The antigen binding proteins may be administered by intermittent therapy, for example for a period of 3 to 6 months and then no dose for 3 to 6 months, followed by administration of antigen binding proteins again for 3 to 6 months, and so on in a cycle.

The dosage may be determined or adjusted by measuring the amount of circulating anti-myostatin antigen binding proteins after administration in a biological sample by using anti-idiotypic antibodies which target the anti-myostatin antigen binding proteins. Other means of determining or adjusting dosage may be utilized, including but not limited to biologic markers (‘biomarkers’) of pharmacology, measures of muscle mass and/or function, safety, tolerability, and therapeutic response. The antigen binding protein can be administered in an amount and for a duration effective to down-regulate myostatin activity in the subject.

The antigen binding protein may be administered to the subject in such a way as to target therapy to a particular site. For example, the antigen binding protein may be injected locally into muscle, for example skeletal muscle.

The antigen binding protein may be used in combination with one or more other therapeutically active agents, for example Mortazapine (Remeron, Zispin: Organon), Megestrol acetate (Megace: BMS), Dronabinol (Marinol: Solvay Pharmaceutical Inc.), Oxandrolone (Oxandrin: Savient), testosterone, recombinant growth hormone (for example Somatropin (Serostim: Serono), Nutropin (Genentech), Humatrope (Lilly), Genotropin (Pfizer), Norditropin (Novo), Saizen (Merck Serono), and Omnitrope (Sandoz)), Cyproheptadine (Periactin: Merck), ornithine oxoglutarate (Cetornan), Methylphenidate (Ritalin: Novartis), and Modafinil (Provigil: Cephalon), orlistat (alli: GSK), sibutramine (Meridia, Reductil), rimonabant (Acomplia, Monaslim, Slimona), used in the treatment of the diseases described herein. Such combinations may be used in the treatment of diseases where myostatin contributes to the disease or where neutralising the activity of myostatin will be beneficial.

When the antigen binding protein is used in combination with other therapeutically active agents, the individual components may be administered either together or separately, sequentially or simultaneously, in separate or combined pharmaceutical formulations, by any appropriate route. If administered separately or sequentially, the antigen binding protein and the therapeutically active agent(s) may be administered in any order.

The combinations referred to above may be presented for use in the form of a single pharmaceutical formulation comprising a combination as defined above optionally together with a pharmaceutically acceptable carrier or excipient.

When combined in the same formulation it will be appreciated that the components must be stable and compatible with each other and the other components of the formulation and may be formulated for administration. When formulated separately they may be provided in any convenient formulation, for example in such a manner as known for antigen binding proteins in the art.

When in combination with a second therapeutic agent active against the same disease, the dose of each component may differ from that when the antigen binding protein is used alone. Appropriate doses will be readily appreciated by those skilled in the art.

The antigen binding protein and the therapeutically active agent(s) may act synergistically. In other words, administering the antigen binding protein and the therapeutically active agent(s) in combination may have a greater effect on the disease, disorder, or condition described herein than the sum of the effect of each alone.

The pharmaceutical composition may comprise a kit of parts of the antigen binding protein together with other medicaments, optionally with instructions for use. For convenience, the kit may comprise the reagents in predetermined amounts with instructions for use.

The terms “individual”, “subject” and “patient” are used herein interchangeably. The subject is typically a human. The subject may also be a mammal, such as a mouse, rat or primate (e.g. a marmoset or monkey). The subject can be a non-human animal. The antigen binding proteins may also have veterinary use. The subject to be treated may be a farm animal for example, a cow or bull, sheep, pig, ox, goat or horse or may be a domestic animal such as a dog or cat. The animal may be any age, or a mature adult animal. Where the subject is a laboratory animal such as a mouse, rat or primate, the animal can be treated to induce a disease or condition associated with muscle wasting, myopathy, or muscle loss.

Treatment may be therapeutic, prophylactic or preventative. The subject may be one who is in need thereof. Those in need of treatment may include individuals already suffering from a particular medical disease in addition to those who may develop the disease in the future.

Thus, the antigen binding protein described herein can be used for prophylactic or preventative treatment. In this case, the antigen binding protein described herein is administered to an individual in order to prevent or delay the onset of one or more aspects or symptoms of the disease. The subject can be asymptomatic. The subject may have a genetic predisposition to the disease. A prophylactically effective amount of the antigen binding protein is administered to such an individual. A prophylactically effective amount is an amount which prevents or delays the onset of one or more aspects or symptoms of a disease described herein.

The antigen binding protein described herein may also be used in methods of therapy. The term “therapy” encompasses alleviation, reduction, or prevention of at least one aspect or symptom of a disease. For example, the antigen binding protein described herein may be used to ameliorate or reduce one or more aspects or symptoms of a disease described herein.

The antigen binding protein described herein is used in an effective amount for therapeutic, prophylactic or preventative treatment. A therapeutically effective amount of the antigen binding protein described herein is an amount effective to ameliorate or reduce one or more aspects or symptoms of the disease. The antigen binding protein described herein may also be used to treat, prevent, or cure the disease described herein.

The antigen binding protein described herein may have a generally beneficial effect on the subject's health, for example it can increase the subject's expected longevity.

The antigen binding protein described herein need not affect a complete cure, or eradicate every symptom or manifestation of the disease to constitute a viable therapeutic treatment. As is recognised in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, is sufficient.

Me disorder, disease, or condition include sarcopenia, cachexia, muscle-wasting, disuse muscle atrophy, HIV, AIDS, cancer, surgery, burns, trauma or injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's syndrome, acute respiratory distress syndrome, steroid induced muscle wasting, myositis and scoliosis.

Age-related muscle wasting (also called myopathy), or sarcopenia, is the progressive loss of muscle mass and muscle strength that occurs with age. This condition is thought to be a consequence of decreased muscle synthesis and repair in addition to increased muscle breakdown. In age-related muscle wasting the bundles of muscle fibers can shrink because individual fibers are lost. Furthermore, due to disuse muscle atrophy in such subjects, muscle fibers also get smaller. Treatments may reverse this muscle atrophy. Thus, the antigen binding proteins described herein may be used to treat sarcopenia.

Age-related muscle wasting begins at middle age and accelerates throughout the remainder of life. The most commonly used definition for the condition is appendicular skeletal mass/height² (kg/m²) less than two standard deviations below the mean value for young adults. This disorder can lead to decreased mobility, functional disability and loss of independence.

Disuse muscle atrophy can be associated with a number of different conditions, diseases or disorders, for example immobilization, post-operative surgery, dialysis, critical care (e.g. burns, ICU), trauma or injury to muscle or bone. Disuse atrophy can result from numerous causes or incidents which lead to prolonged periods of muscle disuse. Muscle atrophy involves the decrease in size and/or number and/or function of muscle fibers.

Cachexia is a condition which is associated with any one or a combination of loss of weight, loss of muscle mass, muscle atrophy, fatigue, weakness and loss of appetite in an individual not actively trying to lose weight. Cachexia can be associated with various other disorders, including any one of the diseases mentioned herein. For example, cachexia may be associated with cancer, infection (for example by HIV or AIDS), renal failure, autoimmunity, and drug or alcohol addiction. Furthermore, cardiac cachexia may be treated using the antigen binding proteins described herein, for example in patients who have experienced myocardial infarction or patients with congestive heart failure. Thus, a patient with cancer cachexia may be treated by the antigen binding proteins described herein.

Chronic obstructive pulmonary disease (COPD) patients may display mild, moderate or severe symptoms of the disease. COPD includes patients with emphysema and bronchitis. Patients with emphysema are generally very thin or frail, and their disease is generally considered to be irreversible. Therefore, the antigen binding proteins described herein can be used to treat patients with emphysema since it is more difficult to improve the patient's underlying lung function. Patients with bronchitis are generally more robust, although they may also lack muscle, and their disease is thought to have some degree of reversibility. Therefore, the antigen binding proteins described herein can be used to treat patients with bronchitis, optionally in combination with treatment of the patient's underlying lung function. Treatment with the antigen binding proteins described herein can have a direct effect on improving the function of muscles involved in respiration in patients with emphysema or bronchitis.

Cancer patients often display muscle wasting which can lead to hospitalization, infection, dehydration, hip fracture, and ultimately death. For example, a 10% loss of muscle mass can be associated with a dramatically inferior prognosis of the cancer patient. Treatment with the antigen binding proteins described herein may improve the performance status of the cancer patient, for example to allow full chemotherapy or a more aggressive use of chemotherapy, and to improve patient quality of life. Thus the antigen binding proteins described herein may be used to treat cancer cachexia.

Cancer includes, for example, prostate, pancreatic, lung, head and neck, colorectal cancer and lymphoma. For example in prostate cancer, the subject may have metastatic prostate cancer and/or may be undergoing androgen deprivation therapy (ADT). Subjects with cancer may have locally advanced or metastatic cancer, for example early stage metastatic cancer. Thus a patient undergoing ADT following prostate cancer may be treated by the antigen binding proteins of the invention.

Patients with chronic renal failure (CRF) or end stage renal disease (ESRD) may be treated with the antigen binding proteins described herein. For example, patients may be treated pre-dialysis to delay the start of dialysis. Alternatively, patients who have been on dialysis for 1 year or more, 2 years or more, or 3 years or more may be treated with the antigen binding proteins described herein. Use of the antigen binding proteins described herein may prevent or treat muscle wasting in the short term, or long-term via chronic use of the antigen binding proteins.

Examples of trauma or injury to muscle, bone or nerve include hip fractures and acute knee injuries. Patients with hip fractures often have muscle atrophy prior to fracture and muscle wasting is a key contributor to hip fracture in many patients. Following hip fracture, muscle and strength is lost due to disuse, and often hip fracture patients do not return to pre-fracture levels of ambulation or function. Furthermore, many hip fracture patients are also afflicted with conditions such as COPD, ESRD and cancer, which can contribute to significant muscle wasting and predispose them to hip fracture. Therefore, patients may be treated with the antigen binding proteins described herein if they are at risk of hip fracture. There is considerable therapeutic urgency associated with hip fracture patients since these patients must be operated on immediately. Therefore, post operative treatment with the antigen binding proteins described herein can help aid the recovery of hip fracture patients by diminishing the loss of muscle mass and strength, and/or improving the recovery of muscle mass and strength. A subject at risk of hip fracture or a subject with a hip fracture may be treated by the antigen binding protein of the invention.

Antigen binding proteins described herein can help to treat elective surgery patients to build muscle in the patient prior to surgery.

Muscular dystrophy refers to a group of genetic, hereditary muscle diseases that cause progressive muscle weakness. Muscular dystrophies are characterized by progressive skeletal muscle weakness, defects in muscle proteins, and the death of muscle cells and tissue. Examples of muscular dystrophies include Duchenne (DMD), Becker, limb-girdle (LGMD), congenital, facioscapulohumeral (FSHD), myotonic, oculopharyngeal, distal, and Emery-Dreifuss. For example the antigen binding proteins described herein can be used to treat Duchenne, Becker or limb-girdle muscular dystrophies. Also, diffuse muscle atrophy rather than local atrophy may be treated by the antigen binding proteins described herein. In particular, myotonic dystrophy may be treated by the antigen binding proteins described herein because of more focalized muscle atrophy/dysfunction and the role of skeletal/bone and cardiac issues in the disease.

Obesity is a condition in which excess body fat has accumulated to such an extent that health may be negatively affected. It is commonly defined as a body mass index (BMI=weight divided by height squared) of 30 kg/m² or higher. This distinguishes obesity from overweight which is defined by a BMI of between 25-29.9 kg/m². Obesity can be associated with various diseases, including cardiovascular diseases, diabetes mellitus type 2, obstructive sleep apnea, cancer, and osteoarthritis. As a result, obesity has been found to reduce life expectancy. Typical treatments for obesity include dieting, physical exercise and surgery. Obesity may be treated by the antigen binding proteins described herein which increase muscle mass and as a result can increase basal metabolic rates. For example, improved serum chemistries and insulin sensitivity may result from such treatment.

Typical aspects or symptoms of decreases in muscle mass, muscle strength, and muscle function include any one or any combination of general weakness, fatigue, reduction in physical activities, vulnerability to falls, functional disability, loss of autonomy, depression due to decreasing mobility, loss of appetite, malnutrition, and abnormal weight loss.

The disease may be associated with high levels of myostatin. The antigen binding proteins described herein can be used to modulate the level of myostatin and/or the activity of myostatin.

Multiple endpoints can be used to demonstrate changes in muscle mass, muscle strength, and muscle function. Such endpoints include the Short Physical Performance Battery, Leg Press, a directed quality of life survey, activities of daily living (ADLs), functional independence measure (FIM), functional tests and scales (e.g. walk test, stair climb, cycle ergometer), strength tests and scales (e.g. hand grip test, manual muscle testing scales), bioimpedance analysis, electromyogram, dynamometer, dual-energy X-ray absorptiometry, computed tomography tests, magnetic resonance imaging, muscle biopsy, muscle histology, blood/biochemistry tests, anthropometry, skin thickness measurements, body mass index assessment, and weight monitoring. Muscle strength can be assessed using bilateral limb muscles, neck muscles or abdominal muscles.

Short Physical Performance Battery (SPPB) is a multi-component measure of lower extremity function that is assessed by measures of standing balance, walking speed, and ability to rise from a chair, rated on a scale of 0-4. Walk test is an assessment of lower extremity function that times how long it takes a patient to walk a certain distance. Leg Press measures leg strength using weights and assessment of force. Multiple scales and systems are used in the art to qualitatively assess a patient's quality of life. Dual-energy X-ray absorptiometry (DEXA) is a measure of estimated skeletal muscle mass.

A number of assays in animals can also be used to demonstrate changes in muscle mass and muscle strength, and muscle function. For example, the grip strength test measures an animal's strength when pulled against a grip strength meter. The inclined plane test measures an animal's ability to suspend itself. The swim test measures functional ability through a representative activity, for example swimming, and is similar to the walk test in humans. The Hindlimb Exertion Force Test (HEFT) measures the maximum force exerted following applied tail stimulus. Other physical performance tests in animals include walking speed and wheel running. These tests/models can be used alone or in any combination.

A High Fat Diet (HFD) induced insulin resistance mouse model may be used as a model for obesity.

Glucocorticoids are commonly used in the treatment of a vast array of chronic inflammatory illnesses, such as systemic lupus erythematosus, sarcoidosis, rheumatoid arthritis, and bronchial asthma. However, administration of high doses of glucocorticoids causes muscle atrophy in human and animals. Similarly, hypercortisolism plays a major role in muscle atrophy in Cushing's disease. Dexamethasone (dex)-induced muscle atrophy is associated with a dose-dependent marked induction of muscle myostatin mRNA and protein expression (Ma K, et al. 2003 Am J Physiol Endocrinol Metab 285:E363-E371). Increased myostatin expression has been also reported in several models of muscle atrophy such as immobilization and burn injuries, in which glucocorticoids play a major role (Lalani R, et al. 2000 J Endocrinol 167:417-428; Kawada S, et al. 2001 J Muscle Res Cell Motil 22:627-633; and Lang C H, et al. 2001 FASEB J 15:NIL323-NIL338). Therefore, a mouse model of glucocorticoid-induced muscle wasting may be used to study the antigen binding proteins of the invention.

Human disuse muscle atrophy commonly occurs in association with orthopedic disorders such as chronic osteoarthritis of a joint or cast immobilization for treatment of fracture as well as in situations of prolonged bed rest for other medical or surgical reasons. Disuse muscle atrophy results in reduced muscle strength and disability. Physical rehabilitation remains the only treatment option, and it is often required for long periods and does not always restore the muscle to normal size or strength. Therefore, a mouse model using sciatic nerve crush to induce muscle atrophy may be used to study the antigen binding proteins of the invention.

A significant portion of cancer patient suffers from weight loss due to progressive atrophy of adipose tissue and muscle wasting. It is estimated that about 20% of cancer deaths are caused by muscle loss. Muscle wasting is generally a good predictor of mortality in many diseases conditions. Data from research on AIDS, starvation and cancer indicate that loss of more than 30-40% of individual pre-illness lean body mass is fatal (DeWye W D. In Clinics in Oncology. Edited by Calman K C and fearon K C H. London: Saunders, 1986, Vol. 5, no 2, p. 251-261; Kotter D P, et al. 1990 J Parent Enteral Nutr 14:454-358; and Wigmore S J, et al. 1997 Br J Cancer 75:106-109). Thus, the possible mitigation of muscle atrophy through the inhibition of signalling pathways involved in muscle wasting is very appealing. Therefore, a C-26 tumour bearing mouse model may be used to study the antigen binding proteins of the invention.

In the clinic, tenotomy refers to surgical transection of a tendon due to congenital and/or acquired deformations in the myotendinous unit, although loss of tendon continuity may also occur during trauma or degenerative musculoskeletal diseases. Tenotomy results in an immediate loss of resting tension, sarcomere shortening, and subsequent decreases in muscle mass and force generation capacity (Jamali et al. 2000 Muscle Nerve 23: 851-862). Therefore, a mouse tenotomy model which induces skeletal muscle atrophy may be used to study the antigen binding proteins of the invention.

The antigen binding proteins described can be used for acute, chronic, and/or prophylactic therapy. Acute therapy can quickly build strength and bring the patient to an adequate level of functional ability that could then be maintained by exercise or chronic therapy. Chronic therapy could be used to maintain or slowly build muscle strength over time. Prophylactic therapy could be used to prevent the declines in muscle mass and strength that typically occur over time in the patient populations described. Improvement of muscle function is not always necessary to define successful treatment since early intervention in less severe muscle wasting requires only maintenance of muscle function.

The antigen binding proteins described may also have cosmetic uses for increasing muscle strength, mass and function. The antigen binding proteins described may also have uses during space flight and training exercises for astronauts.

The antigen binding proteins described may have a direct biological effect on muscle, such as skeletal muscle. Alternatively, the antigen binding proteins described may have an indirect biological effect on muscle, such as skeletal muscle.

For example, the antigen binding proteins may have an effect on one or more of muscle histology, muscle mass, muscle fibre number, muscle fibre size, muscle regeneration and muscle fibrosis. For example muscle mass may be increased. In particular, lean mass of a subject may be increased. The mass of any one or a combination of the following muscles: quadriceps, triceps, soleus, tibialis anterior (TA), and extensor digitorum longus (EDL); may be increased. The antigen binding proteins described may increase muscle fibre number and/or muscle fibre size. The antigen binding proteins described may enhance muscle regeneration and/or reduce muscle fibrosis. The antigen binding proteins described may increase the proliferation rate of myoblasts and/or activate myogenic differentiation. For example, the antigen binding proteins may increase the proliferation and/or differentiation of muscle precursor cells.

The antigen binding proteins described may have one or a combination of the following effects on satellite cells: activate, increase proliferation and promote self renewal. The antigen binding proteins described may modulate myostatin levels. The antigen binding proteins described may increase body weight of the subject. The antigen binding proteins described may increase muscle contractility and/or improve muscle function. The antigen binding proteins may increase bone density.

The antigen binding proteins described herein may modulate the synthesis and/or catabolism of proteins involved in muscle growth, function and contractility. For example protein synthesis of muscle-related proteins such as myosin, dystrophin, myogenin may be upregulated by use of the antigen binding proteins described herein. For example protein catabolism of muscle-related proteins such as myosin, dystrophin, myogenin may be downregulated by use of the antigen binding proteins described herein.

### Diagnostic Methods of Use

The antigen binding proteins described herein may be used to detect myostatin in a biological sample in vitro or in vivo for diagnostic purposes. For example, the anti-myostatin antigen binding proteins can be used to detect myostatin in cultured cells, in a tissue or in serum. The tissue may have been first removed (for example a biopsy) from a human or animal body. Conventional immunoassays may be employed, including ELISA, Western blot, immunohistochemistry, or immunoprecipitation.

By correlating the presence or level of myostatin with a disease, one of skill in the art can diagnose the associated disease. Furthermore, detection of increased levels of myostatin in a subject may be indicative of a patient population that would be responsive to treatment with the antigen binding proteins described herein. Detection of a reduction in myostatin levels may be indicative of the biological effect of increased muscle strength, mass and function in subjects treated with the antigen binding proteins described herein.

The antigen binding proteins may be provided in a diagnostic kit comprising one or more antigen binding proteins, a detectable label, and instructions for use of the kit. For convenience, the kit may comprise the reagents in predetermined amounts with instructions for use.

### Gene Therapy

Nucleic acid molecules encoding the antigen binding proteins described herein may be administered to a subject in need thereof. The nucleic acid molecule may express the CDRs in an appropriate scaffold or domain, the variable domain, or the full length antibody. The nucleic acid molecule may be comprised in a vector which allows for expression in a human or animal cell. The nucleic acid molecule or vector may be formulated for administration with a pharmaceutically acceptable excipient and/or one or more therapeutically active agents as discussed above.

### Examples

### 1. Generation of Recombinant Proteins

### 1.1 Purification of Mature Dimeric Myostatin

The HexaHisGB1Tev/(D76A) mouse myostatin polyprotein sequence (SEQ ID NO: 101) was expressed in a CHO secretion system. The GB1 tag (SEQ ID NO: 102) is described in WO2006/127682 and was found to enable the expression of myostatin at higher levels and enabled the proper folding of myostatin compared with constructs which used an Fc tag. The mouse polyprotein sequence (SEQ ID NO: 103) was used to generate the mature myostatin sequence (SEQ ID NO: 104) because the sequences of human and mouse mature myostatin are 100% identical. To reduce any potential degradation of myostatin, the mouse polyprotein sequence was engineered with a D76A mutation in the region “DVQRADSSD”.

The expressed HexaHisGB1Tev/(D76A) mouse myostatin polyprotein, minus the signal sequence, was captured from the CHO medium using Ni-NTA agarose (Qiagen) in 50 Tris-HCl buffer, pH8.0 with 0.5M NaCl. The Ni eluate was buffer exchanged into Furin cleavage buffer (50 mM HEPES, pH 7.5, 0.1M NaCl, 0.1% Triton X-100, 1 mM CaCl₂), followed by cleavage by Furin (expressed in-house, sequence of Furin shown in SEQ ID NO: 105) at 1:25 V/V of Furin/protein ratio, overnight at room temperature. Furin cleaves polyprotein between the pro-peptide and mature myostatin (between “TPKRSRR” and “DFGLDCD”) to generate pro-peptide and mature myostatin.

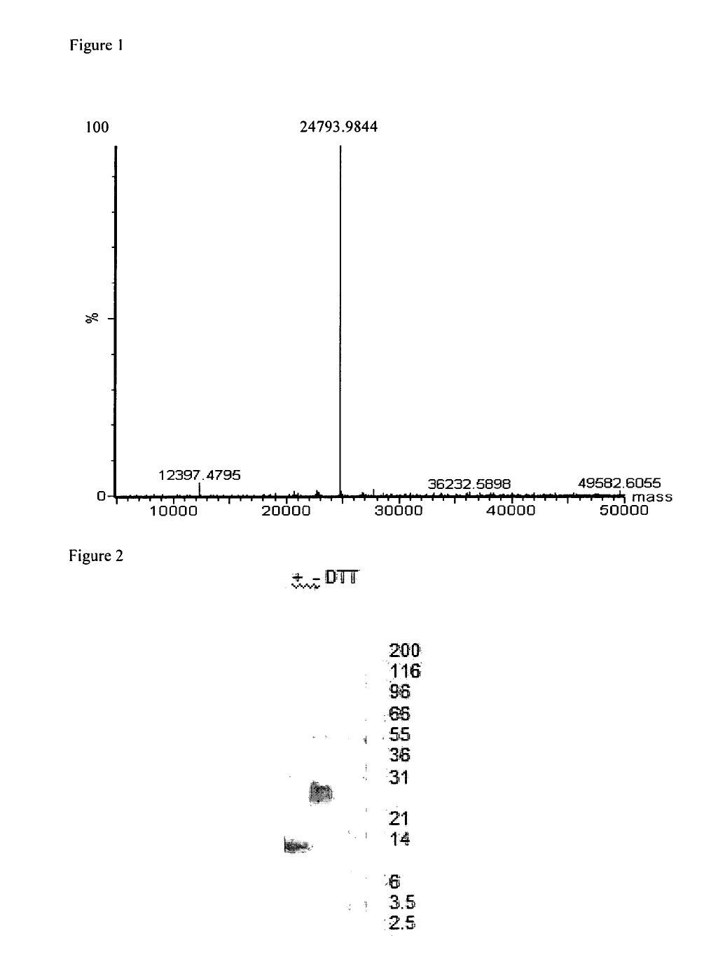

The whole mixture of the Furin cleavage reaction was put into 6M Gdn-HCl to dissociate the aggregate. Mature myostatin was isolated from the mixture using C8 RP-HPLC (Vydac 208TP, Grace, Deerfield, Ill., USA) at 60° C. with 15-60% buffer B gradient in 40 minutes (C8 RP-HPLC buffer A: 0.1% TFA in H₂O, buffer B: 0.1% TFA in 100% Acetonitrile). The fractions in the front of the peak, which contain mature myostatin, were pooled and used for subsequent in vitro assays. FIG. 1 shows the LC/MS analysis for mature myostatin and FIG. 2 shows a NuPAGE gel with the reduced and non-reduced myostatin samples.

### 1.2 In Vitro Biological Activity of Recombinant Myostatin

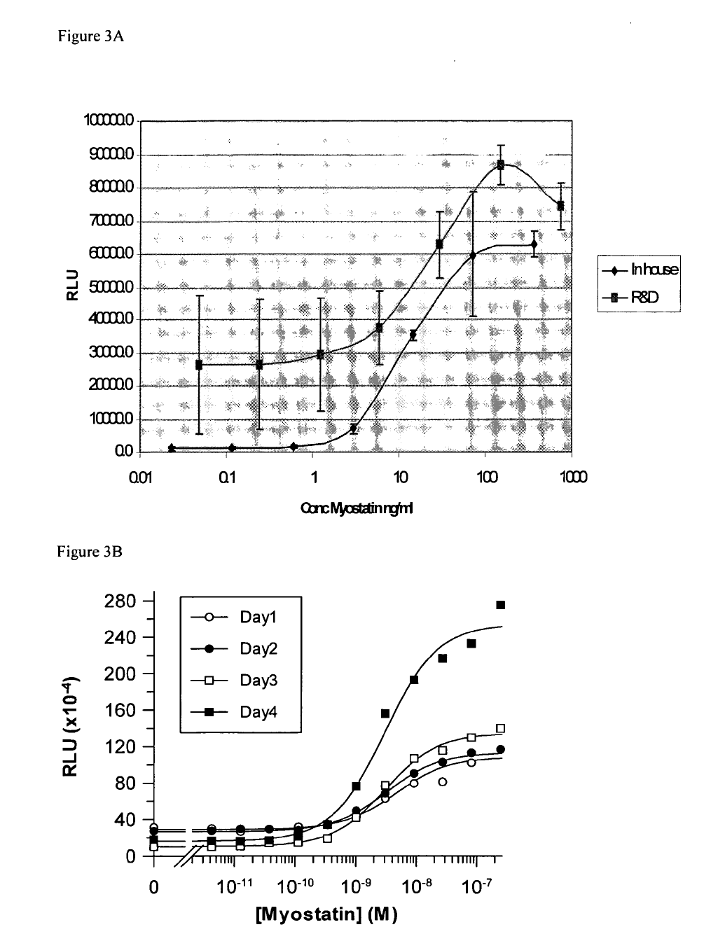

The myostatin responsive reporter gene assay (Thies et al., (2001) Growth Factors 18(4) 251-259) was used to assess in vitro activity of myostatin in Rhabdomyosarcoma cells (A204). A204 cells (LGC Promochem HTB-82) were grown in DMEM high glucose without phenol red (Invitrogen), 5% charcoal stripped FCS (Hyclone) and 1× Glutamax (Invitrogen). Cells were then trypsinised to generate a suspension and transfected with a pLG3 plasmid containing a luciferase gene under the control of 12×CAGA boxes of the PAI-1 promoter using Gemini transfection reagent (in-house reagent, described in patent WO2006/053782). Cells were seeded at 40,000 cells per well of a 96 well Fluoronunc Plate (VWR) and allowed to settle and grow overnight. The following day, recombinant mature myostatin, either R&D Systems myostatin (788-G8-010/CF) or in-house myostatin (as described above at 1.1), both having the sequence shown in SEQ ID NO: 104, was added to the medium of each well by serial dilution and cells were left to incubate for a further 6 hours prior to the addition of SteadyLite (Perkin Elmer LAS) which was incubated at room temperature for 20 minutes and read in a SpectraMax M5 reader (Molecular Devices). Dose response curves demonstrating myostatin activation of cell signalling, resulting in luciferase expression are shown in FIG. 3A. It can clearly be seen that both the R&D Systems and in-house mature dimeric myostatin species activate A204 cells resulting in luciferase signal in a dose dependent manner. The in-house purified myostatin demonstrates a preferential lower background in the assay and improved dynamic range over the R&D Systems myostatin.

In an alternative method, A204 cells (LGC Promochem HTB-82) were grown in McCoys media (Invitrogen) and 10% heat inactivated FBS (Invitrogen). Cells were then detached with a 1:1 mixture of versene (Invitrogen) and TrypLE (Invitrogen) and resuspended in DMEM high glucose without phenol red, 5% charcoal-stripped serum (Hyclone) and 2 mM glutamax (Invitrogen) (Assay Media). 14×10⁶ cells were transfected by mixing 18.2 μg of pLG3 plasmid—containing a luciferase gene under the control of 12×CAGA boxes of the PAI-1 promoter—with 182 μl of 1 mM Gemini transfection reagent (in-house reagent, described in patent WO2006/053782) in suspension. Cells were transferred into a T175 culture flask and incubated overnight. The following day, recombinant myostatin, either R&D Systems myostatin (788-G8-010/CF) or in-house myostatin (as described above at 1.1), was added to 96 well, black FluoroNUNC assay plate (VWR) either by serial dilution or at a constant concentration in the presence of a serial dilution of test antibody in a final volume of 20 μl. Myostatin antibody mixtures were allowed to preincubate for 30 minutes. The transfected cells were detached from flasks with versene:TrypLE, resuspended in assay media at 2.2×10⁵ cells/ml and dispensed into the assay plate at 180 μl/well. Plates were incubated for a further 6 hours prior to the addition of 50 μl of SteadyLite reagent (Perkin Elmer LAS) which was incubated at room temperature for 20 minutes and read in a SpectraMax M5 reader (Molecular Devices). Dose response curves demonstrating mature dimeric myostatin activation of cell signalling, resulting in luciferase expression are shown in FIG. 3B. The in-house myostatin species activates A204 cells resulting in luciferase signal in a dose dependent manner and reproducibly on different test occasions as represented by data obtained on different days.

### 2. Generation of Monoclonal Antibodies and Characterisation of Mouse Monoclonal Antibody 10B3

### 2.1 Monoclonal Antibodies

SJL/J mice (Jackson Laboratories) were immunised by intraperitoneal injection each with mature myostatin (prepared as described above in Example 1.1). Before immunisation, the myostatin was conjugated to C. parvum and mice immunised with the conjugate (2.5 μg myostatin conjugated to 10 μg C. parvum) and a further 7.5 μg of soluble myostatin. Spleen cells from the mice were removed and B lymphocytes fused with mouse myeloma cells derived from P3X63BCL2-13 cells (generated in-house, see Kilpatrick et al., 1997 Hybridoma 16(4) pages 381-389) in the presence of PEG1500 (Boehringer) to generate hybridomas. Individual hybridoma cell lines were cloned by limiting dilution (using the method described in E Harlow and D Lane). Wells containing single colonies were identified microscopically and supernatants tested for activity.

Initially, hybridoma supernatants were screened for binding activity against recombinant myostatin in an FMAT sandwich assay format. A secondary screen of these positives was completed using a BIAcore™ method to detect binding to recombinant myostatin (R&D Systems, 788-G8-010/CF) and in-house expressed purified myostatin (see 1.1 above).

Positives identified from the myostatin binding assay were subcloned by limiting dilution to generate stable monoclonal cell lines. Immunoglobulins from these hybridomas, grown in cell factories under serum free conditions, were purified using immobilised Protein A columns. These purified monoclonal antibodies were then re-screened for myostatin binding by ELISA and BIAcore™

Monoclonal antibody 10B3 was identified as a potent antibody that bound to recombinant myostatin.

### 2.2 Sequencing of Monoclonal Antibody 10B3 and Cloning of the 10B3 Chimera

Total RNA was extracted from the 10B3 hybridoma cells and the cDNA of the heavy and light variable domains was produced by reverse transcription using primers specific for the leader sequence and the antibody constant regions according to the pre-determined isotype (IgG2a/κ). The cDNA of the variable heavy and light domains was then cloned into a plasmid for sequencing. The 10B3 V_{H} region amino acid sequence is shown in SEQ ID NO: 7. The 10B3 V_{L} region amino acid sequence is shown in SEQ ID NO: 8. The Kabat CDR sequences for 10B3 are shown in Table 3 and Table 4.

TABLE 3
Heavy chain CDR sequences

| Antibody | CDR H1 | CDR H2 | CDR H3 |
|---|---|---|---|
| 10B3 | GYFMH | NIYPYNGVSNYNQ | RYYYGTGPADWYFD |
| | (SEQ ID | RFKA | V |
| | NO: 1) | (SEQ ID NO: 2) | (SEQ ID NO: 3) |

TABLE 4
Light chain CDR sequences

| Anti- | | | |
|---|---|---|---|
| body | CDR L1 | CDR L2 | CDR L3 |
| 10B3 | KASQDINSYLS | RANRLVD | LQCDEFPLT |
| | (SEQ ID NO: 4) | (SEQ ID NO: 5) | (SEQ ID NO: 6) |

A chimeric antibody was constructed by taking variable regions from the 10B3 murine monoclonal antibody (V_{H}: SEQ ID NO: 7; V_{L}: SEQ ID NO: 8) and grafting these on to human IgG1/k wild type constant regions. A signal sequence (as shown in SEQ ID NO: 9) was used in the construction of these constructs.

In brief, the cloned murine variable regions were amplified by PCR to introduce restriction sites required for cloning into mammalian expression vectors (R1d_Ef1 and R1n_Ef1). Hind III and Spe I sites were designed to frame the V_{H} domain and allow cloning into a vector (R1d_Ef1) containing the human γ1 wild type constant region. Hind III and BsiW I sites were designed to frame the V_{L} domain and allow cloning into a vector (R1n_Ef1) containing the human κ constant region. Clones with the correct V_{H} (SEQ ID NO: 25) and V_{L} (SEQ ID NO: 8) sequences were identified and plasmids prepared (using standard molecular biology techniques) for expression in CHOK1 cell supernatants. Antibodies were purified from the cell supernatant using immobilised Protein A columns and quantified by reading the absorbance at 280 nm.

The resulting chimeric antibody was termed 10B3 chimera (10B3C or HCLC). The 10B3 chimeric antibody has a heavy chain amino acid sequence as set out in SEQ ID NO: 26. The 10B3 chimeric antibody has a light amino acid sequence as set out in SEQ ID NO: 27.

### 2.3 Binding to Recombinant Myostatin

10B3 and 10B3 chimera (10B3C) bound myostatin (R&D Systems, 788-G8-010/CF) in a sandwich ELISA. Plates were coated with myostatin at 10 ng/well and blocked with Block solution (PBS, 0.1% TWEEN and 1% BSA). Following washing (PBS, 0.1% TWEEN), antibody was incubated at 37° C. for 2 hours over a dilution series and plates washed again prior to incubation at 37° C. for 1 hour with anti-mouse HRP or anti-human HRP (Dako, P0161 & Sigma, A-8400, respectively). Plates were again washed and OPD substrate (Sigma, P9187) added until colourometric reaction occurred and the reaction stopped by the addition of H₂SO₄. Plates were read at an absorbance of 490 nm and EC50 determined (see Table 5).

TABLE 5
EC50 of parental 10B3 and chimeric 10B3 antibodies

| Antibody | Mean EC50 (ng/ml) | 95% confidence levels (ng/ml) |
|---|---|---|
| 10B3 | 69 | 46-102 |
| 10B3 Chimera | 49 | 33-73 |

The affinity of 10B3 mouse parental and 10B3C for recombinant myostatin was assessed by BIAcore™ (surface plasmon resonance) analysis. Analysis was carried out by the use of a capture surface: anti-mouse IgG was coupled to a C1 chip by primary amine coupling for 10B3 mouse parental; and a protein A surface was created on a C1 chip by primary amine coupling for 10B3 chimera.

After capture, recombinant myostatin was passed over the surface at 64 nM, 16 nM, 4 nM, 1 nM, 0.25 nM and 0.0625 nM, with a buffer injection (i.e. 0 nM) used for double referencing. There was a regeneration step between each analyte injection, after which the new antibody capture event occurred before the next injection of myostatin. The data was analysed using both the 1:1 model and the Bivalent model inherent to the T100 machines analysis software (see Table 6). Both capture surfaces could be regenerated using 100 mM phosphoric acid, the work was carried out using HBS-EP as the running buffer and using 25° C. as the analysis temperature.

TABLE 6
T100 data for parental 10B3 and chimeric
10B3 binding to myostatin

| | | Equilibrium Constant |
|---|---|---|
| | Equilibrium Constant | (KD) for 10B3 Mouse |
| Kinetic Model | (KD) for 10B3 Chimera | Parental |
| All Curves 1:1 Model | 88 pM | 1 nM |
| All Curves Bivalent | 3.6 nM | 5.9 nM |
| Model | | |

To further analyse the binding capability of 10B3, ELISA based assays were undertaken to determine whether binding was specific for pure mature myostatin or if binding could still occur with other myostatin antigens including latent complex, and mature myostatin released from latent complex following BMP-1 cleavage between Arg 75 and Asp 76 of the myostatin pro-peptide (Wolfman et al (2003) PNAS 100: pages 15842-15846).

Purification of human myostatin pro-peptide was carried out using a HexaHisGB1Tev/Human Myostatin pro-peptide sequence (SEQ ID NO: 106). This sequence was expressed in the CHO secretion system, and expressed protein was captured by Ni-NTA (GE Healthcare, NJ) from the CHO medium. The HexaHisGB1tag was cleaved by Tev protease (expressed in-house, sequence shown in SEQ ID NO: 107). Tev protease cleaves between the tag and the pro-peptide (between “ENLYFQ” and “ENSEQK”) of SEQ ID NO: 106 to yield the sequence of SEQ ID NO: 108.

The cleaved tag and non-cleaved hexaHisGB1Tev/Human Myostatin polyprotein were captured on Ni-NTA in the presence of 6M Guanidine HCL, with the tag cleaved human myostatin polyprotein in the unbound flowthrough. The flowthrough was applied on Superdex 200 column (GE Healthcare, NJ) in 1×PBS buffer and the aggregated, dimer and monomer forms were separated on the column. The human myostatin pro-peptide (SEQ ID NO: 108) dimer form was used in latent complex formation.

Myostatin latent complex was prepared by mixture of the purified human myostatin pro-peptide (SEQ ID NO: 108) and mature myostatin (SEQ ID NO: 104) in 6M Guanidine HCl at 3:1(w/w) ratio for 2 hours at room temperature, followed by dialysis into 1×PBS overnight at 4° C., and loaded onto Superdex 200 (GE Healthcare, NJ) in 1×PBS buffer. The fractions in the peak which contained both myostatin pro-peptide and mature myostatin were pooled. The latent complex was confirmed by both LC/MS and SDS-PAGE (data not shown). For the BMP-1 digestion, 150 μl of human myostatin latent complex (1.5 mg/ml) was incubated with 225 μl of BMP-1 (0.217 mg/ml), 75 μl of 25 mM HEPES (pH 7.5) and 150 μl of: 20 mM CaCl2, 4 μM ZnCl₂ and 0.04% Brij 35. The reaction was incubated at 30° C. overnight. BMP-1 protein was expressed in-house (sequence shown in SEQ ID NO: 111) using a CHO secretion system.

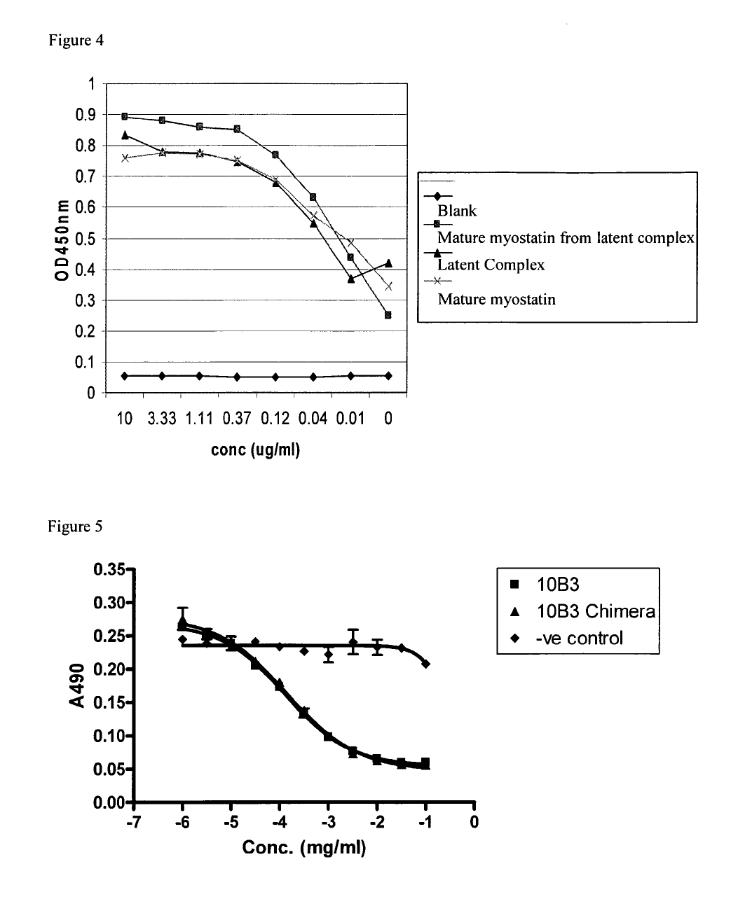

The myostatin antigens were coated onto wells of an EIA/RIA plate (Costar) at 100 ng/well at 4° C. overnight in PBS, prior to blocking (PBS, 3% BSA) for 30 minutes at room temperature. Plates were washed (PBS, 1% BSA and 0.1% Tween20), prior to the addition of a dilution series of 10B3 in wash buffer and incubation for 2 hours at room temperature. Plates were again washed prior to addition of peroxidase-conjugated Affinipure F(ab′)2 fragment donkey anti-mouse IgG (Jackson Laboratories cat 715-036-151) diluted 1:10,000 in wash buffer and incubated for 1 hour at room temperature. A final wash step preceded addition of TMB substrate and colorimetric change which was stopped with Sulphuric acid and plates read at 450 nm. FIG. 4 shows that 10B3 is able to bind mature dimeric myostatin, latent complex (tetramer), and myostatin released from the latent complex following BMP-1 cleavage. It was also found that 10B3 does not bind to the pro-peptide dimer (data not shown).

### 2.4 Crude Mapping of the 10B3 Binding Epitope on Myostatin

Biotinylated 14 mer peptides overlapping by 10 amino acids (offset by 4 amino acids) were synthesised based on the myostatin amino acid sequence to map the location of the binding epitope recognised by 10B3 (supplied by Mimotopes, Australia).

Work was carried out on an SRU BIND reader (SRU Biosystems). A streptavidin biosensor plate was washed, a baseline reading taken, and biotinylated peptides captured onto the streptavidin coated biosensor plate. The plate was washed again, and a new baseline reading taken, antibody was then added and binding monitored.

The details of the 14 mer custom designed artificial peptide sequences, overlapping by 10 amino acids (offset by 4 amino acids) are provided in Table 7.

TABLE 7
myostatin artificial peptides

| Pep- | | | | | |
|---|---|---|---|---|---|
| tide | | | | | |
| No | NTerm | Sequence | CTerm | Hydro | MWt |
| 1 | H— | DFGLDCDEHSTESRGSG | —NH2 | −0.045 | 2164.84 |
| | | (SEQ ID NO: 56) | | | |
| 3 | Biotin- | SGSGDCDEHSTESRCCRY | —NH2 | 0.118 | 2217.09 |
| | | (SEQ ID NO: 57) | | | |
| 5 | Biotin- | SGSGHSTESRCCRYPLTV | —NH2 | 0.346 | 2165.17 |
| | | (SEQ ID NO: 58) | | | |
| 7 | Biotin- | SGSGSRCCRYPLTVDFEA | —NH2 | 0.394 | 2173.18 |
| | | (SEQ ID NO: 59) | | | |
| 9 | Biotin- | SGSGRYPLTVDFEAFGWD | —NH2 | 0.456 | 2229.16 |
| | | (SEQ ID NO: 60) | | | |
| 11 | Biotin- | SGSGTVDFEAFGWDWIIA | —NH2 | 0.646 | 2183.13 |
| | | (SEQ ID NO: 61) | | | |
| 13 | Biotin- | SGSGEAFGWDWIIAPKRY | —NH2 | 0.505 | 2265.28 |
| | | (SEQ ID NO: 62) | | | |
| 15 | Biotin- | SGSGWDWIIAPKRYKANY | —NH2 | 0.416 | 2337.39 |
| | | (SEQ ID NO: 63) | | | |
| 17 | Biotin- | SGSGIAPKRYKANYCSGE | —NH2 | 0.183 | 2113.11 |
| | | (SEQ ID NO: 64) | | | |
| 19 | Biotin- | SGSGRYKANYCSGECEFV | —NH2 | 0.286 | 2182.15 |
| | | (SEQ ID NO: 65) | | | |
| 21 | Biotin- | SGSGNYCSGECEFVFLQK | —NH2 | 0.436 | 2180.17 |
| | | (SEQ ID NO: 66) | | | |
| 23 | Biotin- | SGSGGECEFVFLQKYPHT | —NH2 | 0.447 | 2211.21 |
| | | (SEQ ID NO: 67) | | | |
| 25 | Biotin- | SGSGFVFLQKYPHTHLVH | —NH2 | 0.593 | 2279.36 |
| | | (SEQ ID NO: 68) | | | |
| 27 | Biotin- | SGSGQKYPHTHLVHQANP | —NH2 | 0.279 | 2183.14 |
| | | (SEQ ID NO: 69) | | | |
| 29 | Biotin- | SGSGHTHLVHQANPRGSA | —NH2 | 0.218 | 2037.94 |
| | | (SEQ ID NO: 70) | | | |
| 31 | Biotin- | SGSGVHQANPRGSAGPCC | —NH2 | 0.297 | 1909.85 |
| | | (SEQ ID NO: 71) | | | |
| 33 | Biotin- | SGSGNPRGSAGPCCTPTK | —NH2 | 0.238 | 1901.87 |
| | | (SEQ ID NO: 72) | | | |
| 35 | Biotin- | SGSGSAGPCCTPTKMSPI | —NH2 | 0.468 | 1905.96 |
| | | (SEQ ID NO: 73) | | | |
| 37 | Biotin- | SGSGCCTPTKMSPINMLY | —NH2 | 0.582 | 2115.27 |
| | | (SEQ ID NO: 74) | | | |
| 39 | Biotin- | SGSGTKMSPINMLYFNGK | —NH2 | 0.39 | 2157.27 |
| | | (SEQ ID NO: 75) | | | |
| 41 | Biotin- | SGSGPINMLYFNGKEQII | —NH2 | 0.504 | 2193.28 |
| | | (SEQ ID NO: 76) | | | |
| 43 | Biotin- | SGSGLYFNGKEQIIYGKI | —NH2 | 0.434 | 2199.26 |
| | | (SEQ ID NO: 77) | | | |
| 45 | Biotin- | SGSGGKEQIIYGKIPAMV | —NH2 | 0.416 | 2060.17 |
| | | (SEQ ID NO: 78) | | | |
| 47 | Biotin- | SGSGIIYGKIPAMVVDRC | —NH2 | 0.558 | 2091.25 |
| | | (SEQ ID NO: 79) | | | |
| 49 | Biotin- | SGSGGKIPAMVVDRCGCS | —OH | 0.396 | 1950.02 |
| | | (SEQ ID NO: 80) | | | |

Analysis of the 14 mer peptide binding data demonstrated that 10B3 was unable to bind any linear epitope within myostatin. Control anti-myostatin antibodies however, were shown to bind epitopes within the peptide set (data not shown).

Subsequent analysis of the myostatin binding site of 10B3C using Pepscan, Chemically Linked Immunogenic Peptides on Scaffolds (CLIPS) technology, suggest that the “PRGSAGPCCTPTKMS” amino acid sequence of myostatin may be the binding site for the chimeric antibody (data not shown).

### 2.5 Neutralisation of Myostatin ActRIIb Receptor Binding

Recombinant soluble ActRIIb (R&D Systems 339-RBB) was coated in wells of an ELISA plate at 1 μg/ml in carbonate buffer overnight at 4° C. Plates were blocked (see Block solution above at 2.3) and washed following standard ELISA protocols. In parallel, 2 nM biotinylated myostatin (in-house, as described in 1.1, biotinylated material) was pre-incubated with an antibody dilution series consisting of 10B3, 10B3C, and a negative control (IgG1 isotype control) for 2 hours at 37° C. The biotinylated myostatin:antibody reactions were then added to the ActRIIb coated plate for 1 hour at 37° C. Standard wash procedures were followed prior to addition of 1:1000 diluted streptavidin-HRP conjugate (Dako P0397) and a further 37° C. incubation for 1 hour. Plates were again washed and assayed at absorbance 490 nm following OPD substrate (Sigma) and acid stop solution treatment Inhibition curves and IC50 values for the inhibition of myostatin activity are shown in FIG. 5 and Table 8 respectively.

TABLE 8
IC50 of ActRIIb receptor neutralisation

| Antibody | Mean IC50 (ng/ml) | 95% confidence levels (ng/ml) |
|---|---|---|
| 10B3 | 132 | 99-176 |
| 10B3 Chimera | 138 | 97-196 |

The receptor neutralisation assay is the most sensitive method available for differentiating molecules with IC50s lower than 1 nM on the basis of potency. It is, however, itself sensitive to the precise concentration of binding-competent biotinylated myostatin. Hence on different occasions other IC50 values have been determined for 10B3 using the same methodology, for example 0.13 nM, 0.108 nM, 0.109 nM, or 0.384 nM (note that in Table 8, 132 ng/ml=0.88 nM).

### 2.6 Inhibition of Biological Activity of Myostatin In Vitro

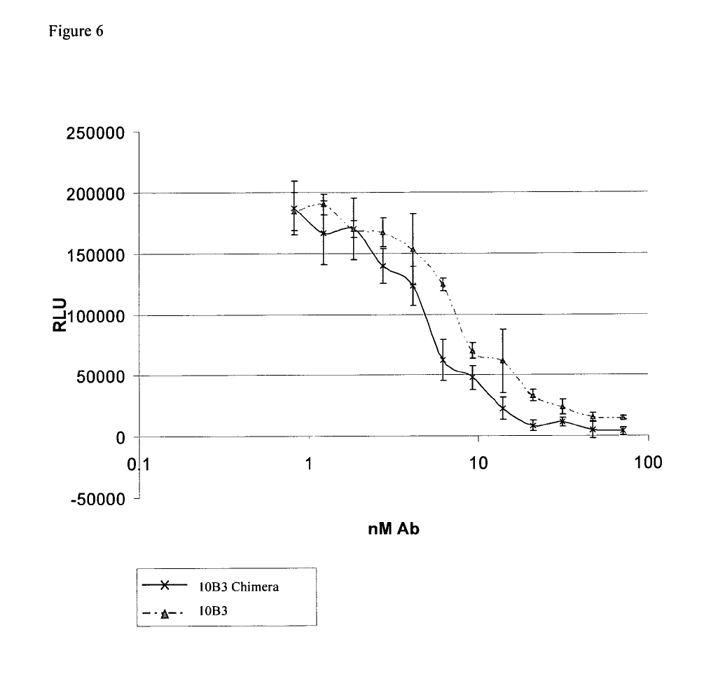

The myostatin responsive reporter gene assay, described above at 1.2, was used to assess the in vitro effect of anti-myostatin antibodies on the activity of myostatin. The assay was modified so that myostatin at a concentration of 2.8 nM (equivalent to ED70 in cell activation assays) was pre-incubated with varying concentrations of 10B3 or 10B3C antibody (0.1-20 nM) at 37° C. prior to addition to transfected A204 cells. Luciferase readouts were performed, from which the inhibition curves shown in FIG. 6 were generated. Table 9 shows the IC50 values determined for the antibodies following 3 repeats of the assay and ANOVA analysis. The data clearly demonstrate a dose dependant inhibition of myostatin activation of the A204 muscle cell line, whereas the control antibody showed no inhibition of myostatin activity.

TABLE 9
IC50 of in vitro myostatin responsive
reporter gene assay (A204 cells)

| Antibody | Mean IC50 (nM) | 95% confidence levels (nM) |
|---|---|---|
| 10B3 | 10.0 | 6.5-15.5 |
| 10B3 Chimera | 6.2 | 3.9-9.9 |

### 2.7 In Vivo Efficacy of 10B3

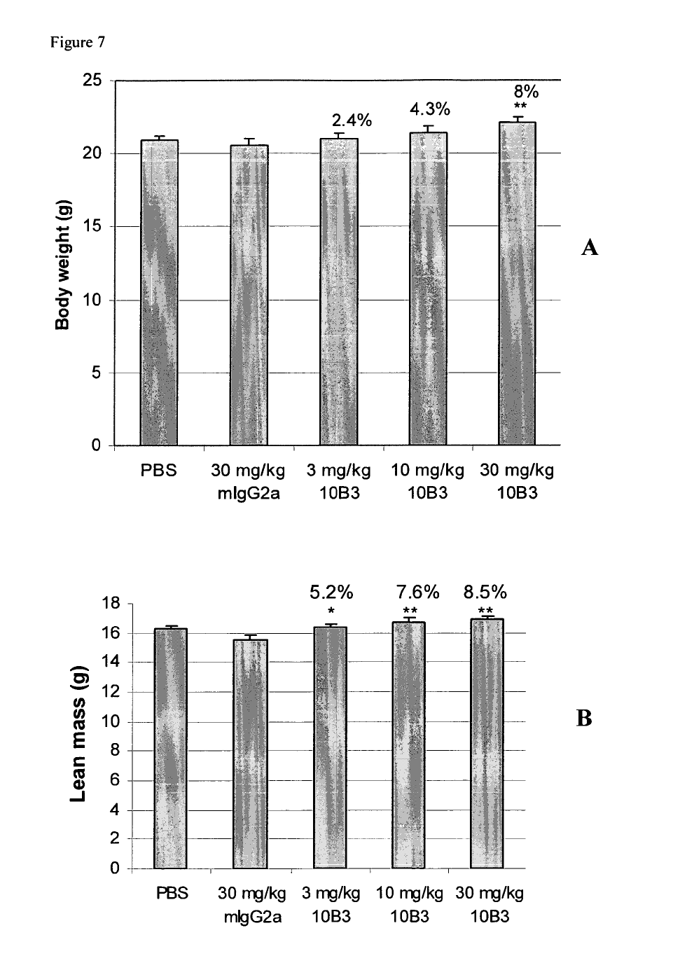

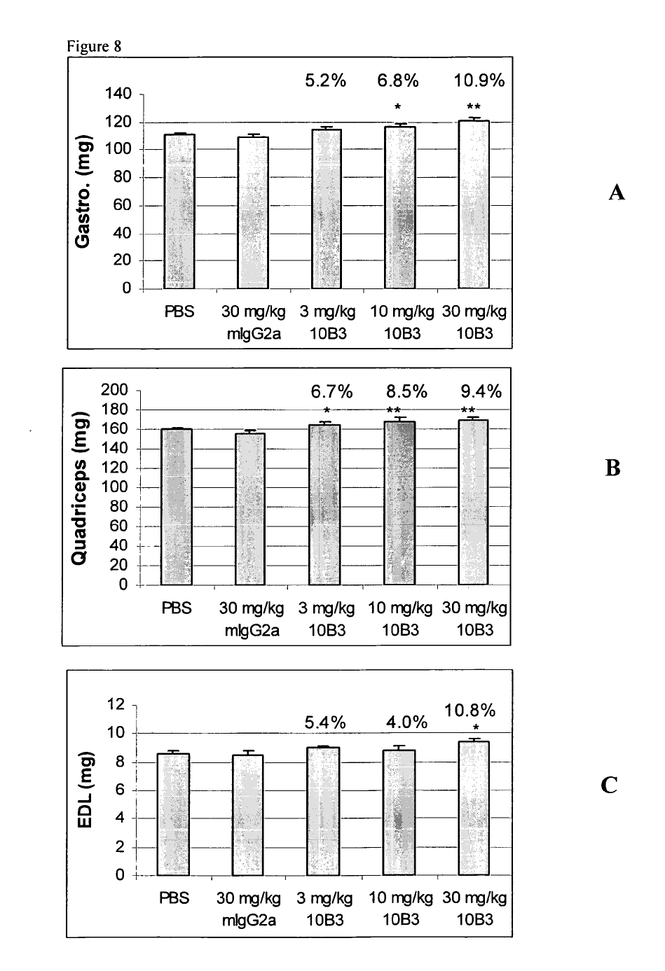

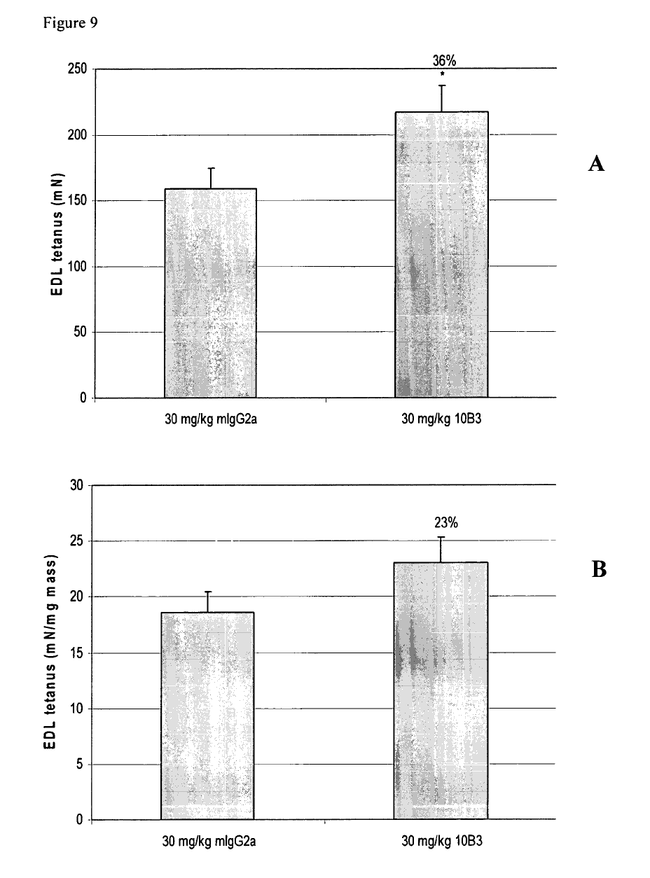

To demonstrate efficacy of parental 10B3, a 35 day study in 8 week old female CB17 SCID mice was undertaken for 5 weeks. Treatment groups (10 animals per group) were dosed on days 1, 4, 8, 15, 22, and 29 by intraperitoneal injection with either, 3, 10 or 30 mg/kg 10B3, whilst control groups received either PBS or isotype control antibody (IgG2a). Upon completion of the study, total body weight (A) and total lean muscle mass (B) of animals were determined by weighing animals and QMRI analysis respectively (FIG. 7). Upon culling of animals (day 35) individual muscles (gastrocnemius (A), quadriceps (B), and extensor digitorum longus (EDL) (C)) were dissected from animals for mass determination (FIG. 8). To determine effects on muscle function ex vivo contractility testing was performed on EDL muscles (FIG. 9), in which tetanic force was determined for muscle (FIG. 9A) and the tetanic force per milligram of muscle mass (FIG. 9B).

A clear dose dependant response to 10B3 was observed in the treatment groups with the 30 mg/kg dose representing the most significant improvement in body weight and lean muscle mass (8% and 8.5%, respectively) following the 35 day study. Analysis of muscle mass demonstrated the same trend with the gastrocnemius, quadriceps and EDL all showing dose dependant increases in mass, again with the mg/kg dosing groups showing greatest significance.

Also, studies (not described) have demonstrated that significant improvement in grip strength can not be seen at an early time point such as 35 days. However, the ex vivo contractility testing demonstrates that significant improvement can be demonstrated in tetanic force measures of the EDL. Furthermore the improvement was demonstrated to be independent of muscle mass. Thus 10B3 exhibits the ability to improve the function of existing muscle mass.

### 3. Humanisation of 10B3

### 3.1 Sequence Analysis

A comparison was made between the sequences of the 10B3 variable regions and other murine and human immunoglobulin sequences. This was done using the FASTA and BLAST programs and by visual inspection.

A suitable human acceptor framework for the 10B3 V_{H} was identified (IGHV1_{—}18 and the JH3 human J segment sequence): SEQ ID NO: 10. A suitable human acceptor framework for the 10B3 V_{L} was identified (IGKV1_{—}16 and the JK2 human J segment sequence): SEQ ID NO: 11. In SEQ ID NO: 10, CDRH1 and CDRH2 of the acceptor framework are present, and CDRH3 is represented by XXXXXXXXXX. In SEQ ID NO: 11, CDRL1 and CDRL2 of the acceptor framework are present, and CDRL3 is represented by XXXXXXXXXX. (The 10 X residues are a placeholder for the location of the CDR, and is not a measure of the number of amino acid sequences in each CDR).

In CDR grafting, it is typical to require one or more framework residues from the donor antibody to be included in place of their orthologues in the acceptor frameworks in order to obtain satisfactory binding. The following murine framework residues in 10B3 were identified as being potentially important in the design of a CDR-grafted (humanised) version of the antibody (position is according to the Kabat et al numbering convention):

| Position (Kabat) | mouse 10B3 V_{H} | Human V_{H} |
|---|---|---|
| 28 | S | T |
| 105 | T | Q |
| Position (Kabat) | mouse 10B3 V_{L} | Human V_{L} |
| 16 | R | G |
| 71 | Y | F |
| 100 | A | Q |

Three humanised V_{H} constructs with different back-mutations were designed to obtain a humanised antibody with satisfactory activity. These are numbered H0 to H2. H0 (SEQ ID NO: 12) consists of a CDR graft of the 10B3 V_{H} CDRs into the specified acceptor sequence, using the Kabat definition of CDRs. H1 (SEQ ID NO: 13) is identical to H0, but with a back-mutation where the amino acid at position 105 is threonine instead of glutamine. H2 (SEQ ID NO: 14) is identical to H0, but with a back-mutation where the amino acid at position 28 is serine instead of threonine.

Note that for all humanised V_{H} regions (and corresponding heavy chains), the sequence of framework 4 (WGQGTMVTVSS) has been modified, whereby the methionine amino acid residue (Kabat position 108) has been substituted for a leucine amino acid residue. This results from the inclusion of a Spe1 cloning site in the DNA sequences encoding the humanised V_{H} regions.

Four humanised V_{L} constructs with different back-mutations were designed to obtain a humanised antibody with satisfactory activity. These are numbered L0 to L3. L0 (SEQ ID NO: 15) consists of a CDR graft of the 10B3 V_{L} CDRs into the specified acceptor sequence, using the Kabat definition of CDRs. L1 (SEQ ID NO: 16) is identical to L0, but with a back-mutation where the amino acid at position 16 is arginine in place of glycine. L2 (SEQ ID NO: 17) is identical to L0, but with a back-mutation where the amino acid at position 71 is tyrosine in place of phenylalanine L3 (SEQ ID NO: 18) is identical to L0, but with a back-mutation where the amino acid at position 100 is alanine in place of glutamine.

### 3.2 Humanisation of 10B3

Humanised V_{H} and V_{L} constructs were prepared by de novo build up of overlapping oligonucleotides including restriction sites for cloning into R1d Ef1 and R1n Ef1 mammalian expression vectors as well as a signal sequence. Hind III and Spe I restriction sites were introduced to frame the V_{H} domain containing the signal sequence (SEQ ID NO: 9) for cloning into R1d Ef1 containing the human IgG1 wild type constant region. Hind III and BsiW I restriction sites were introduced to frame the V_{L} domain containing the signal sequence (SEQ ID NO: 9) for cloning into R1n Ef1 containing the human kappa constant region. This is essentially as described in WO 2004/014953.

### 4. Expression and Characterisation of Humanised Antibodies

### 4.1 Preparation of Antibodies

Humanised V_{H} constructs (H0, H1 and H2) and humanised V_{L} constructs (L0, L1, L2 and L3) were prepared in R1d_Ef1 and R1n_Ef1 mammalian expression vectors. Plasmid heavy chain-light chain combinations (H0L0, H0L1, H0L2, H0L3, H1L0, H1L1, H1L2, H1L3, H2L0, H2L1, H2L2, H2L3) were transiently co-transfected into CHOK1 cells and expressed at small scale to give twelve different humanised antibodies.

The plasmids for each antibody were transfected into CHOK1 cells in duplicate and in two separate experiments. In addition, 10B3 chimera was expressed as a positive control. Antibodies produced in the CHOK1 cell supernatant were analysed for activity in the myostatin binding ELISA (see 4.2). The ELISA data for just one experiment are illustrated in the graph in FIG. 10A. All twelve humanised mAbs show binding to recombinant myostatin in this ELISA. Across both experiments, the mAbs containing the H2 or L2 chains tended to have better binding activity for myostatin which was similar to that observed for 10B3 chimera.

FIG. 10B is derived from FIG. 10A and displays antibodies containing the H2 and/or L2 chains and 10B3 chimera.

H0L0, H1L2 and H2L2 were selected for larger scale expression, purification and further analysis.

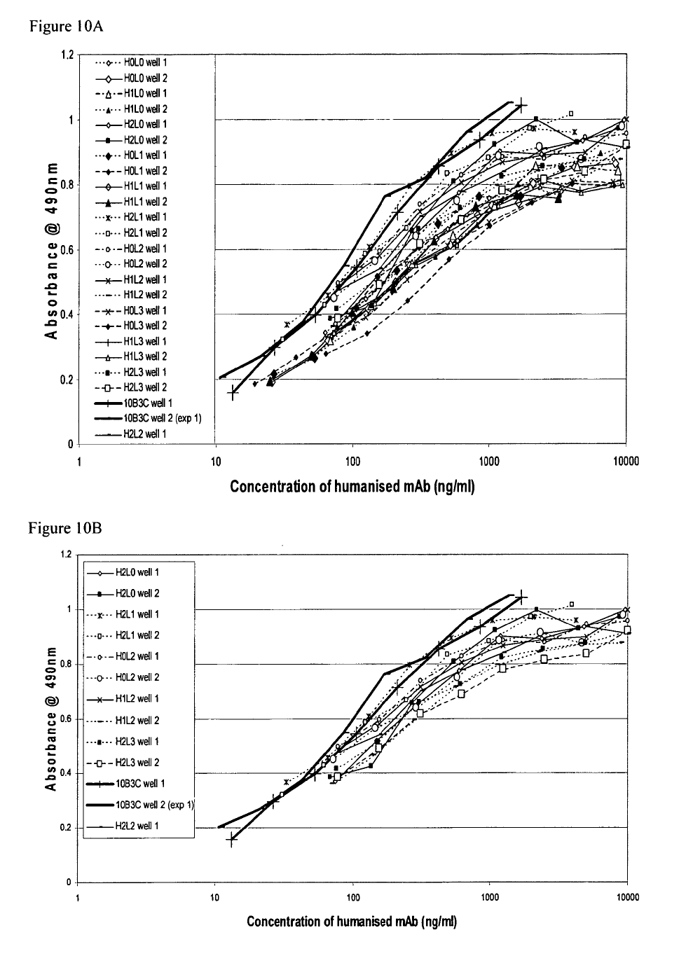

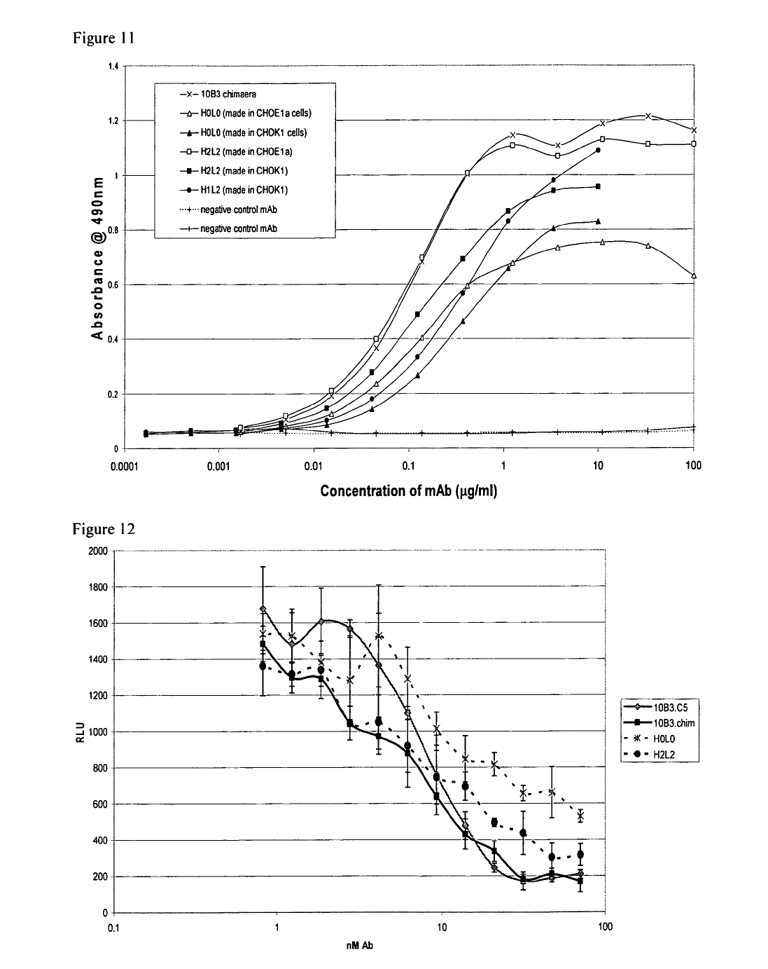

Purified H0L0, H1L2 and H2L2 bound recombinant myostatin by direct ELISA. The method was carried out as described in 4.2 and the ELISA data are illustrated in the graph in FIG. 11. H2L2 and H0L0 were generated in both CHOE1a and CHOK1 cell expression systems. The low concentration of the antibodies obtained from the CHOK1 preparation made accurate quantification difficult. High concentrations of purified antibodies were obtained from the CHOE1a preparation. 10B3 chimeric antibody was included in the ELISA as a positive control (this material was made in CHOE1a). H2L2 binding activity for myostatin was equivalent to 10B3 chimera and better than that observed for H0L0.

### 4.2 Myostatin Binding ELISA

The myostatin binding ELISA was carried out approximately according to this protocol. A 96-well ELISA plate was coated at 4° C. overnight with 10 ng/well recombinant myostatin. This plate was then washed 3-times in wash buffer (PBS, 0.1% Tween-20). The wells were blocked for 1 hour at room temperature with block solution (PBS, 0.1% Tween-20+1% bovine serum albumin [BSA]), before washing 3-times in wash buffer. Antibodies were then titrated out to a suitable concentration range (approximately 100 to 0.001 μg/ml), added to the plate and incubated for 1 hour at room temperature. The plate was then washed 3-times in wash buffer. An anti-mouse IgG HRP-conjugated antibody (P0260 by Dako, this reagent was used according to the manufacturer's instructions) was used to detect binding of mouse antibodies, such as 10B3. An anti-human kappa light chain HRP-conjugated antibody (A7164 by Sigma Aldridge, this reagent was used according to the manufacturer's instructions) was used to detect binding of humanized or chimeric antibodies, such as 10B3 chimera or H0L0. The plate was then washed 3-times in wash buffer and developed with an OPD substrate (from Sigma, used according to the manufacturer's instructions) and read at 490 nm on a plate reader.

### 4.3 Binding to Recombinant Myostatin by Biacore™

Purified H0L0, H1L2 and H2L2 bound recombinant myostatin by BIAcore™ Recombinant myostatin was immobilised at three different densities (low, medium and high, to give R-max values of approximately 35, 120 and 350 RU's respectively) onto a BIAcore™ chip. Antibodies were passed over at 256, 64, 16, 4 and 1 nM. 0 nM antibody was used for double referencing and data was fitted to the 1:1 model.

There are a number of caveats that are applicable to data generated from this assay; immobilising myostatin onto the chip surface may cause a conformation change in the protein, or it may obscure the antibody binding epitope on the protein, and will lead to a heterogeneous surface (possibly generating multiple binding events). Low density immobilisation of myostatin should give 1:1 binding (predominantly), medium and high density immobilisation of myostatin are likely to be affected by bivalent (avidity) binding events. The correct antibody concentration is essential for the determination of accurate values in this assay.

Therefore data generated using the BIAcore™ is generally to be used to rank constructs, rather than to provide definitive kinetics. The BIAcore™ data are illustrated in Tables 10 to 12.

TABLE 10
BIAcore ™ analysis of 10B3 chimera, H0L0, H1L2
and H2L2 binding to a low density myostatin surface

| | On-rate, ka | Off-rate, kd | Binding affinity, |
|---|---|---|---|
| Construct | (Ms⁻¹) | (s⁻¹) | KD (nM) |
| 10B3 chimera | 5.987 × 10⁵ | 9.668 × 10⁻⁴ | 1.615 |
| H0L0 | 8.012 × 10⁵ | 6.615 × 10⁻³ | 8.255 |
| H1L2 | 2.205 × 10⁵ | 3.324 × 10⁻³ | 15.08 |
| H2L2 | 3.206 × 10⁵ | 2.682 × 10⁻³ | 8.366 |

Note:
dissociation phase shortened to approximately 250 seconds for the analysis, to improve curve fitting

TABLE 11
BIAcore ™ analysis of 10B3 chimera, H0L0, H1L2 and
H2L2 binding to a medium density myostatin surface.

| | On-rate, ka | Off-rate, kd | Binding affinity, |
|---|---|---|---|
| Construct | (Ms⁻¹) | (s⁻¹) | KD (nM) |
| 10B3 chimera | 4.129 × 10⁵ | 5.593 × 10⁻⁴ | 1.355 |
| H0L0 | 2.575 × 10⁵ | 9.301 × 10⁻⁴ | 3.612 |
| H1L2 | 1.369 × 10⁵ | 6.932 × 10⁻⁴ | 5.064 |
| H2L2 | 2.456 × 10⁵ | 7.368 × 10⁻⁴ | 3.000 |

Note:
curve fits were generally poor

TABLE 12
BIAcore ™ analysis of 10B3 chimera, H0L0, H1L2 and
H2L2 binding to a high density myostatin surface.

| | On-rate, ka | Off-rate, kd | Binding affinity, |
|---|---|---|---|
| Construct | (Ms⁻¹) | (s⁻¹) | KD (nM) |
| 10B3 chimera | 2.478 × 10⁵ | 2.185 × 10⁻⁴ | 0.882 |
| H0L0 | 1.463 × 10⁵ | 3.375 × 10⁻⁴ | 2.307 |
| H1L2 | 9.224 × 10⁴ | 2.232 × 10⁻⁴ | 2.420 |
| H2L2 | 1.473 × 10⁵ | 2.160 × 10⁻⁴ | 1.467 |

Note:
curve fits were generally poor

These data indicate that binding affinity improves with the increase in myostatin surface density on the BIAcore™ chip, which is likely to be due to avidity binding. However, rank order stays approximately the same and is independent of the surface used to measure affinity (rank order of binding affinity=10B3 chimera>H2L2>H0L0>H1L2). These data are in broad agreement with the myostatin ELISA data.

### 4.4 Neutralisation of Recombinant Myostatin in a Reporter Cell Bioassay

Humanised antibodies were tested in the myostatin responsive reporter gene assay, described above (1.2), to assess in vitro efficacy. Myostatin at a concentration of 2.8 nM was pre-incubated with varying concentrations of antibody (0.1-20 nM) at 37° C. prior to addition to transfected A204 cells and subsequent luciferase readout. The resulting data are shown in FIG. 12 and the determined IC50s (ANOVA analysis) are shown in Table 13.

TABLE 13
IC50 of humanised antibodies in A204 in vitro activity assay

| Antibody | Mean IC50 (nM) | 95% confidence levels (nM) |
|---|---|---|
| 10B3 | 8.5 | 7.2-10.1 |
| 10B3 Chimera | 5.1 | 4.2-6.1 |
| H0L0 | 10.2 | 7.9-13.1 |
| H2L2 | 8.6 | 6.8-10.7 |

The humanised antibodies inhibit myostatin-induced activation of A204 cells, however, compared to the chimeric 10B3 some loss in activity has been observed, possibly due to the effects of the human framework region. Losses in activity are minimal however and are certainly within 2 fold in the assay.

### 5. Developability Analysis of the Humanised Antibodies

In silico analysis for potential deamidation sites in both the heavy and light chains of 10B3 chimera and the humanised antibodies identified asparagine at Kabat position 54 (N54) in heavy chain CDRH2 as having a high potential for deamidation. In order to characterise this residue further, we generated 10B3 chimeric antibodies and humanised H2L2 antibodies where N54 was substituted for aspartate (D) or glutamine (Q) amino acid residues.

The light chain of 10B3 chimera and the humanised antibodies have a cysteine (C) residue at Kabat position 91 in CDRL3. Unpaired cysteines can be chemically reactive leading to modifications during antibody process development, resulting in possible heterogeneity of product and potential variations in affinity. In addition this residue might be able to promote misfolding or aggregation due to mis-pairing with other cysteines in the variable regions which are essential for making the Immunoglobulin fold. In order to characterise this residue further, we generated 10B3 chimeric antibodies and humanised H2L2 antibodies where C91 was substituted for a serine (S) amino acid residue.

In addition, we also combined the deamidation substitutions made in heavy chain CDRH2 with the substitution at position 91 in light chain CDRL3. The antibodies generated as part of these analyses are illustrated in Table 14.

TABLE 14
Humanised antibody variants generated for developability analysis

| | Heavy chain | Light chain |
|---|---|---|
| | variable | variable |
| | region: SEQ | region: SEQ |
| Antibody molecule name | ID NO: | ID NO: |
| 10B3 chimera N54D (HCLC-N54D) | 19 | 8 |
| 10B3 chimera N54Q (HCLC-N54Q) | 20 | 8 |
| 10B3 chimera N54D & C91S (HCLC- | 19 | 21 |
| N54D-C91S) | | |
| 10B3 chimera N54Q & C91S (HCLC- | 20 | 21 |
| N54Q-C91S) | | |
| 10B3 chimera C91S (HCLC-C91S) | 25 | 21 |
| H2L2 N54D (H2L2-N54D) | 22 | 17 |
| H2L2 N54Q (H2L2-N54Q) | 23 | 17 |
| H2L2 N54D & C91S (H2L2-N54D-C91S) | 22 | 24 |
| H2L2 N54Q & C91S (H2L2-N54Q-C91S) | 23 | 24 |
| H2L2 C91S (H2L2-C91S) | 14 | 24 |

### 5.1 Expression and Characterisation of the Developability Variants

The heavy and light chain constructs necessary to express these antibodies were prepared by site directed mutagenesis of the relevant H2 heavy chain and L2 light chain expression vectors. Plasmid heavy chain-light chain combinations (H2L2-N54D; H2L2-N54Q; H2L2-N54D-C91S; H2L2-N54Q-C91S; H2L2-C91S) were transiently co-transfected into CHO cells and expressed at small scale to give five different humanised antibodies. In addition, 10B3 chimera (HCLC) and H2L2 were expressed as positive controls.

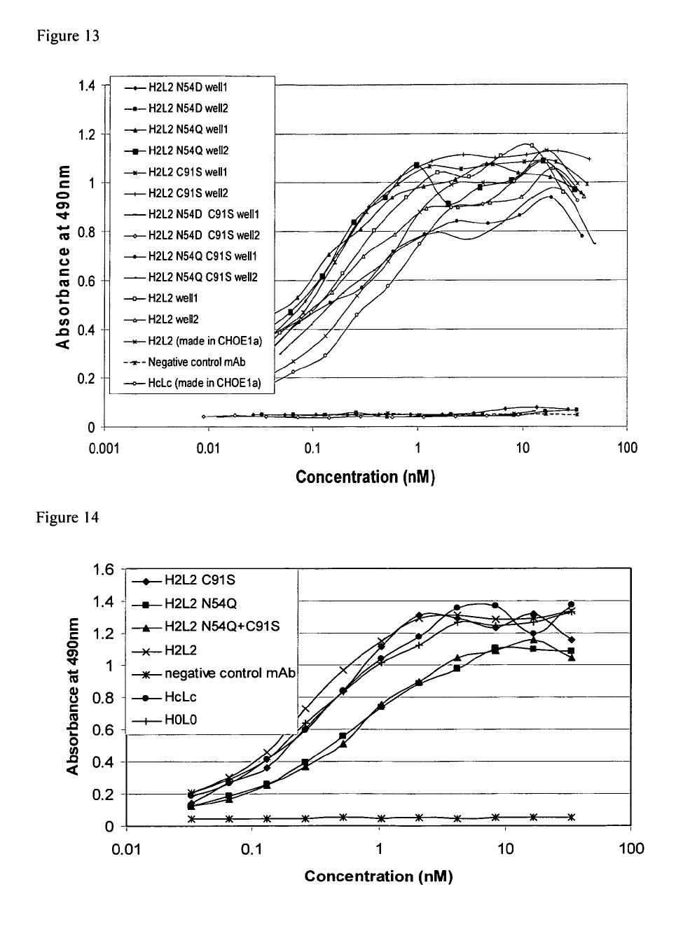

The plasmids for each antibody were transfected into CHOK1 cells in duplicate and in two separate experiments. Antibodies produced in the CHOK1 cell supernatant were analysed for activity in the myostatin binding ELISA. The ELISA method was carried out as described in 4.2 and the ELISA data for just one experiment are illustrated in the graph in FIG. 13. H2L2 mAbs containing the N54Q and/or the C91S substitution showed binding to recombinant myostatin in this ELISA, and this binding was approximately equivalent to 10B3 chimera (HCLC) or H2L2 respectively. 10B3 chimera and H2L2 mAbs containing the N54D substitution alone (or in combination with the C91S substitution) did not bind to recombinant myostatin in this ELISA.

H2L2-N54Q, H2L2-C91S, and H2L2-N54Q C91S were selected for larger scale expression (in both CHOK1 and CHOE1a expression systems), purification and further analysis. These antibodies were analysed for activity in the myostatin binding ELISA. The ELISA method was carried out as described in section 4.2 and the ELISA data for just one experiment (from a total of three) are illustrated in the graph in FIG. 14. H2L2 C91S appeared to have similar binding activity to myostatin as 10B3 chimera, H0L0 and H2L2. However, H2L2 N54Q and H2L2 N54Q C91S appeared to have reduced binding activity for myostatin.

Developability constructs were also tested to determine any changes in myostatin binding affinity by BIAcore using similar methods described above at 4.3 (see Table 15). The data (for low density surface) demonstrate that substitution of the predicted deamidation site (N54Q) results in at least a 2 fold loss in affinity in the H2L2 humanised variant.

TABLE 15
Kinetics of myostatin binding of myostatin developability variants

| Construct | ka | kd | KD (nM) |
|---|---|---|---|
| 10B3 Chimera (HCLC) | 3.323E+5 | 1.477E−3 | 4.44 |
| H2L2 | 3.113E+5 | 3.735E−3 | 12.0 |
| H0L0 | 1.922E+5 | 4.363E−3 | 22.7 |
| H2L2-C91S | 1.903E+5 | 3.153E−3 | 16.6 |
| H2L2-N54Q | 1.590E+5 | 4.447E−3 | 28.0 |
| H2L2-N54Q-C91S | 1.389E+5 | 4.623E−3 | 33.3 |

The affinity of 10B3 mouse parental and H2L2-C91S developability variant for recombinant myostatin was also assessed by FORTEbio™ (bio-layer inferometry) analysis. FORTEbio™ analyses were carried out by antigen capture. Myostatin (in-house, see above at 1.1) was coupled onto amine reactive biosensors by primary amine coupling in accordance with the manufacturer's instructions. Antibodies were then captured onto this surface at 20 nM concentrations. The data was analysed using the evaluation software inherent in the machine and the data analysed using 1:1 fit (see Table 16). Due to the limited number of myostatin molecules bound to the sensor surface and the low antibody concentration, avidity effects are reduced, enabling a more accurate measure of affinity compared to the Biacore analyses. The data show that the parental antibody (10B3) has an affinity of 310 pM whilst the developability variant H2L2-C91S has an affinity of 73 pM. However, due to the nature of the binding of the antibodies to myostatin, these values are mainly used for ranking purposes, and the affinity may not be representative of the affinity in vivo.

TABLE 16
Affinity of 10B3 parental and H2L2-C91S
developability variant for myostatin

| | Molar Conc | k_{d} | kₐ | K_{D} | Assoc |
|---|---|---|---|---|---|
| Antibody | [M] | [1/s] | [1/Ms] | [M] | R² |
| 10B3 parental | 2E−8 | 1.31E−4 | 4.24E5 | 3.10E−10 | 0.9981 |
| H2L2-C91S | 2E−8 | 2.99E−5 | 4.10E5 | 7.30E−11 | 0.99652 |

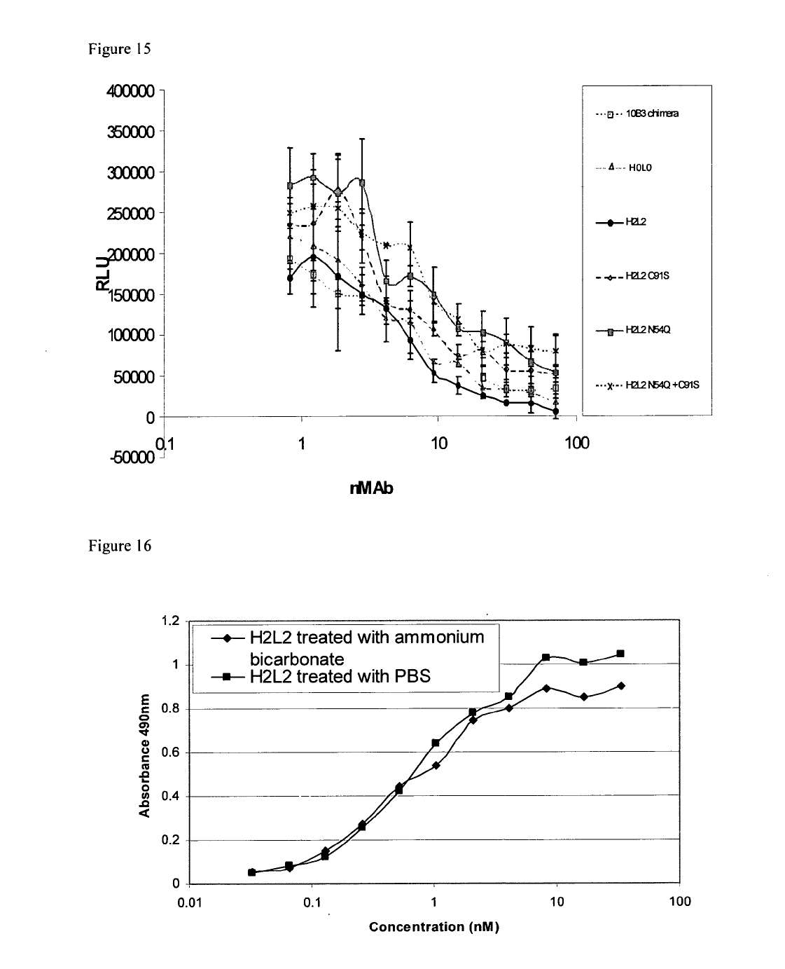

The effect of developability mutations on in vitro neutralisation assays was also undertaken using the A204 luciferase assay described above at 1.2. A graphical representation of inhibition curves is shown in FIG. 15 and corresponding IC50 values are presented in Table 17. The humanised variants have lost no apparent neutralisation potency relative to developability variants according to this assay.

TABLE 17
IC50 of developability antibody variants
in A204 in vitro activity assay

| Antibody | Mean IC50 (nM) | 95% confidence levels (nM) |
|---|---|---|
| 10B3 Chimera | 8.45 | 5.36-13.31 |
| H0L0 | 10.07 | 5.74-17.65 |
| H2L2 | 10.14 | 5.87-17.49 |
| H2L2-C91S | 9.26 | 5.17-16.62 |
| H2L2-N54Q | 11.98 | 6.35-22.59 |
| H2L2-N54Q-C91S | 10.42 | 5.99-18.11 |

### 5.2 Deamidation Potential of the Developability Variants

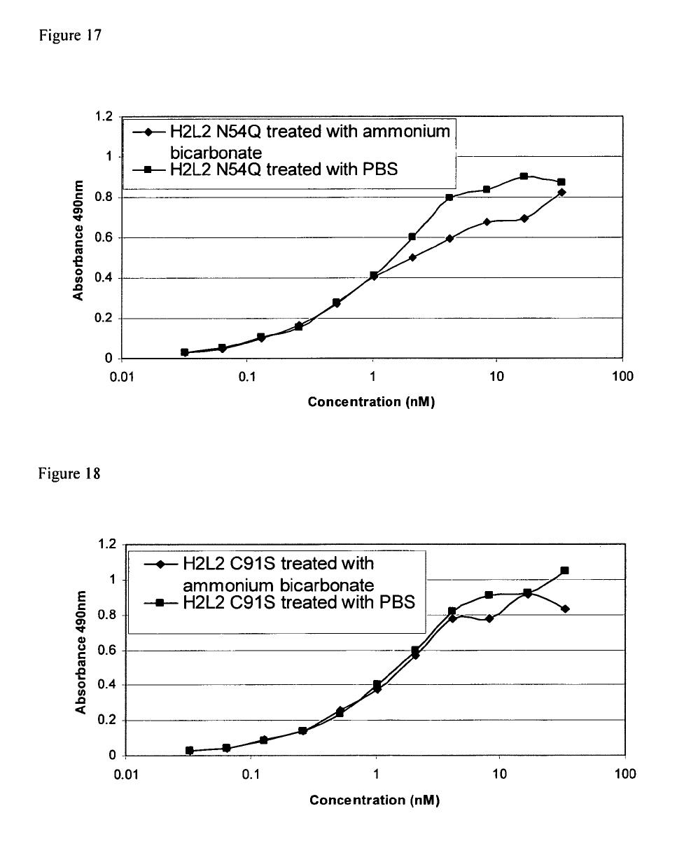

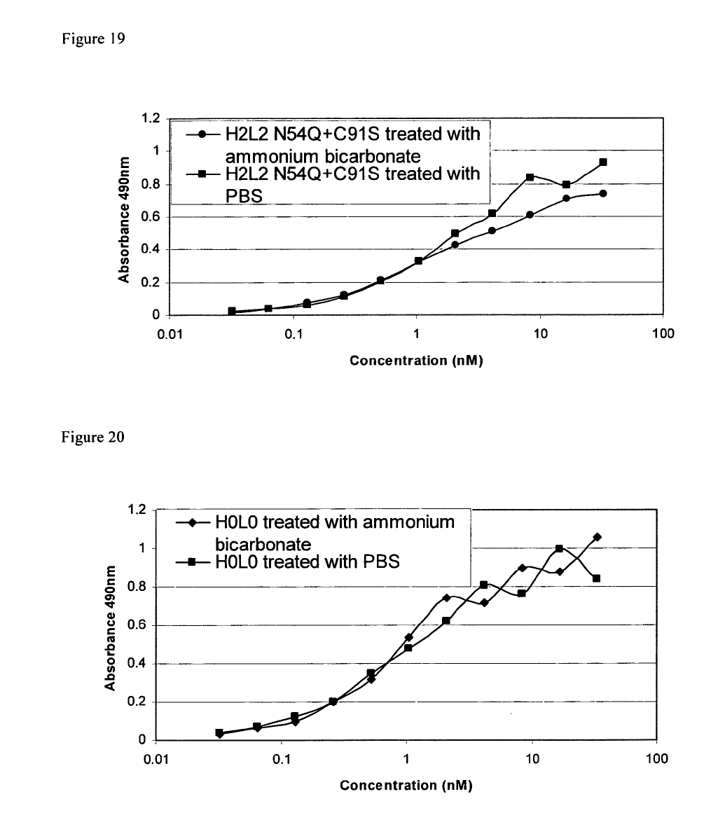

H0L0, H2L2, H2L2-C91S, H2L2-N54Q and H2L2-N54Q-C91S antibodies were subjected to stress conditions that induce deamidation, by incubation with 1% ammonium bicarbonate at pH9.0 at 37° C. for 48 hours. Following treatment, H0L0, H2L2, H2L2-C91S, H2L2-N54Q and H2L2-N54Q-C91S were analysed for functional activity in a myostatin binding ELISA (as described in 4.2). The ELISA data for just one experiment (from a total of two) are illustrated in FIGS. 16 to 20. These data clearly indicate that the treatment procedure did not affect the ability of any of the antibodies to bind to myostatin.

### 6. CDRH3 Variant Humanised Antibodies

### 6.1 Construction of CDRH3 Variant Humanised Antibodies

Site-directed mutagenesis of CDRH3 (SEQ ID NO: 3) of each residue to an alternative amino acid residue was carried out using the antibody H2L2-C91S (variable sequences: SEQ ID NO: 14 and 24 respectively; full-length sequences: SEQ ID NO: 30 and 40 respectively) as a base molecule. Full length DNA expression constructs including human constant regions for the base sequences of H2 and L2-C91S (SEQ ID NO: 45 and 55 respectively) were produced using pTT vectors (National Research Council Canada, with a modified Multiple Cloning Site (MCS)).

Approximately 300 CDRH3 variants were generated and approximately 200 variants were tested in the subsequent analysis (see 6.2 and 6.3).

### 6.2 CDRH3 Variant Expression in HEK 293 6E Cells

pTT plasmids encoding the heavy and light chains respectively of the approximately 200 CDRH3 variants were transiently co-transfected into HEK 293 6E cells and expressed at small scale to produce antibody. The heavy chains have the base sequence of H2 with variant CDRH3 sequences and the light chains have the base sequence of L2-C91S, as described above. Antibodies were assessed directly from the tissue culture supernatant.

### 6.3 Initial Scan-ProteOn XPR36—on Tissue Culture Supernatants

The initial kinetic analyses for the CDRH3 screen were carried out on the ProteOn XPR36 (Biorad Laboratories). For residues R95 to P100_B, analysis was carried out using a Protein A/G capture surface (Pierce 21186) was used and for residues A100_C to V102, an anti-human IgG surface was used (Biacore/GE Healthcare BR-1008-39). Both capture surfaces were prepared similarly using primary amine coupling to immobilise the capture molecule on a GLM chip (Biorad Laboratories 176-5012). CDRH3 variants were captured directly on either the Protein A/G or anti-human IgG surface (depending on the residue mutated) from tissue culture supernatants from transient transfections expressing the particular variant of interest. After capture, in-house recombinant human myostatin (see 1.1 above) was used as an analyte at 256 nM, 32 nM, 4 nM, 0.5 nM and 0.0625 nM, with a buffer injection alone (i.e. 0 nM) used to double reference the binding curves. Following the myostatin binding event, the capture surfaces were regenerated: for the Protein A/G capture surface, 100 mM phosphoric acid was used to regenerate the capture surface; and for the anti-human IgG surface, 3M MgCl₂ was used to regenerate the capture surface; the regeneration removed the previously captured antibody ready for another cycle of capture and binding analysis. The data was then fitted to the 1:1 model (with mass transport) inherent to the ProteOn analysis software. The run was carried out using HBS-EP (Biacore/GE-Healthcare BR-1006-69) and the analysis temperature was 25° C.

The results were difficult to interpret due to the nature of the interaction, since it is unlikely that the 1:1 model adequately describes the interaction, however by judging the sensorgrams it was possible to make a selection of constructs that may have improved affinity over the base molecule. We judged the screen to have identified eleven CDRH3 variants that appeared to have a better kinetic profile than the base molecule. The heavy chains of the eleven CDRH3 variants are described below in Table 18 (using Kabat numbering). All of the variants had the light chain L2-C91S (variable sequence: SEQ ID NO: 24; full-length sequence: SEQ ID NO: 40, full length DNA sequence SEQ ID NO: 55). A further CDRH3 variant that was identified to have a better kinetic profile than the base molecule was F100G_S (SEQ ID NO: 110), but this was not analysed further.

TABLE 18
CDRH3 variant sequences

| Name | Sequence of CDRH3 |
|---|---|
| H2L2-C91S | RYYYGTGPADWYFDV |
| | (SEQ ID NO: 3) |
| H2L2-C91S_Y96L | RLYYGTGPADWYFDV |
| | (SEQ ID NO: 82) |
| H2L2-C91S_G99D | RYYYDTGPADWYFDV |
| | (SEQ ID NO: 83) |
| H2L2-C91S_G99S | RYYYSTGPADWYFDV |
| | (SEQ ID NO: 84) |
| H2L2-C91S_G100A_K | RYYYGTKPADWYFDV |
| | (SEQ ID NO: 85) |
| H2L2-C91S_P100B_F | RYYYGTGFADWYFDV |
| | (SEQ ID NO: 86) |
| H2L2-C91S_P100B_I | RYYYGTGIADWYFDV |
| | (SEQ ID NO: 87) |
| H2L2-C91S_W100E_F | RYYYGTGPADFYFDV |
| | (SEQ ID NO: 88) |
| H2L2-C91S_F100G_N | RYYYGTGPADWYNDV |
| | (SEQ ID NO: 89) |
| H2L2-C91S_F100G_Y | RYYYGTGPADWYYDV |
| | (SEQ ID NO: 90) |
| H2L2-C91S_V102N | RYYYGTGPADWYFDN |
| | (SEQ ID NO: 91) |
| H2L2-C91S_V102S | RYYYGTGPADWYFDS |
| | (SEQ ID NO: 92) |

Reference to the antibodies by code (i.e. H2L2-C91S_Y96L) means the antibody generated by co-transfection and expression of a first and second plasmid encoding the light and heavy chains, for example a plasmid containing the pTT5_H2_Y96L sequence and a plasmid containing the pTT5_L2-C91S sequence in a suitable cell line.

### 6.4 Expression of a Selected Panel of CDRH3 Variants

Heavy and light chains of the eleven CDRH3 variants set out in Table 18 were expressed in HEK 293 6E cells (as described in 6.2), affinity purified using immobilised Protein A columns (GE Healthcare), and quantified by reading absorbance at 280 nm.

### 6.5 Binding to Recombinant Myostatin by BIAcore™

To judge whether the selection of constructs from the initial screen on the ProteOn XPR36 had been successful, an off-rate ranking experiment was performed on purified recombinant antibodies. Myostatin (recombinant in-house, see 1.1 above) was covalently immobilised on a CM5 chip (Biacore/GE Healthcare BR-1000-14) by primary amine coupling at three different densities, low, medium and high, which resulted in surfaces that gave a maximal binding signal of approximately 60 resonance units (RU's), 250 RU's and 1000 RU's respectively with the concentration of antibody used. A single concentration of antibody, 256 nM, was used with a buffer injection to double reference the binding interaction. The initial rate of dissociation (off-rate) was calculated using the software inherent to the Biacore 3000 machine for the interaction of all the antibodies against each density of myostatin surface. Regeneration was by using 100 mM phosphoric acid, and the assay was run using HBS-EP buffer at 25° C.

It was found that all the constructs tested showed a better off-rate (dissociation rate constant) than the base molecule (H2L2 C91S), in that the off rate was slower than H2L2 C91S. On the high density surface the top 5 constructs, excluding the 10B3 chimera were H2L2-C91S_P100B_I, H2L2-C91S_W100E_F, H2L2-C91S_F100G_Y, H2L2-C91S_G99S, and H2L2-C91S_P100B_F.

### 6.6 Full Kinetic Analysis of Binding to Recombinant Myostatin by BIAcore™

Myostatin (recombinant in-house, see 1.1 above) was immobilised on Series S CM5 chip (Biacore/GE Healthcare BR-1006-68) at low, medium and high density which resulted in surfaces that gave a maximal binding signal of approximately 15 RUs, 37 RUs and 500 RUs respectively. The CDRH3 variants were passed over all three surfaces at 256 nM, 64 nM, 16 nM, 4 nM, 1 nM with a buffer injection (i.e. 0 nM) used for double referencing, regeneration was using 100 mM phosphoric acid. The data was fitted to the Bivalent model inherent to the T100 Biacore machine and was run using HBS-EP at 25° C.

In general the fits for the base H2L2-C91S were poor compared to the CDR variants on all three density surfaces, such that an accurate baseline value was hard to obtain. Of the three surfaces, the highest density surface gave the best separation between base antibody and CDR variants, though again the fit for the base H2L2-C91S molecule is poor. However, this surface might be expected to give most separation between the constructs as well as being the surface most likely to provide the best surface for true bivalent binding, since it is likely that avidity binding and rebinding events are more frequent and hence may “magnify” small differences in affinity. In general, all the CDR variants appeared better than the base H2L2-C91S, mainly because of a superior (i.e. slower) off-rate, especially on the high density surface.

Due to the methodology involved in this assay, in covalently coupling the target antigen to the biosensor chip surface, the actual affinities derived may not reflect the affinity that may be seen in vivo. However, this data is useful for ranking purposes. Using the data from the high density surface of this assay, the top 5 constructs, based on overall affinity (equilibrium constant KD) but excluding the chimera 10B3, were F100G_Y, P100B_I, P100B_F, F100G_N and W100E_F. However all other constructs affinities were within two fold of F100G_Y.

### 6.7 Myostatin Capture ELISA

The eleven affinity purified CDRH3 variants were also analyzed for binding activity in the myostatin capture ELISA.

A 96-well ELISA plate was coated at 4° C. overnight with 2.5 μg/ml polyclonal Antibody to Myostatin (R&D Systems AF788). This plate was then washed 3-times in wash buffer (PBS, 0.1% Tween-20) and blocked for 1 hour at room temperature with block solution (PBS, 0.1% Tween-20+1% bovine serum albumin [BSA]). Then, myostatin was added at 1 μg/ml in block buffer during 1 hour followed by 3-times in wash buffer. Antibodies were then titrated out to a suitable concentration range (approximately 10 to 0.01 μg/ml), added to the plate and incubated for 1 hour at room temperature. The plate was then washed 3-times in wash buffer. An anti-human kappa light chain HRP-conjugated antibody (Sigma A7164, used according to the manufacturer's instructions) was used to detect binding of humanized or chimeric antibodies, such as 10B3 chimera (HcLc) or H0L0. The plate was then washed 3-times in wash buffer and developed with an OPD substrate (according to the manufacturer's instructions) and read at 490 nm on a plate reader.

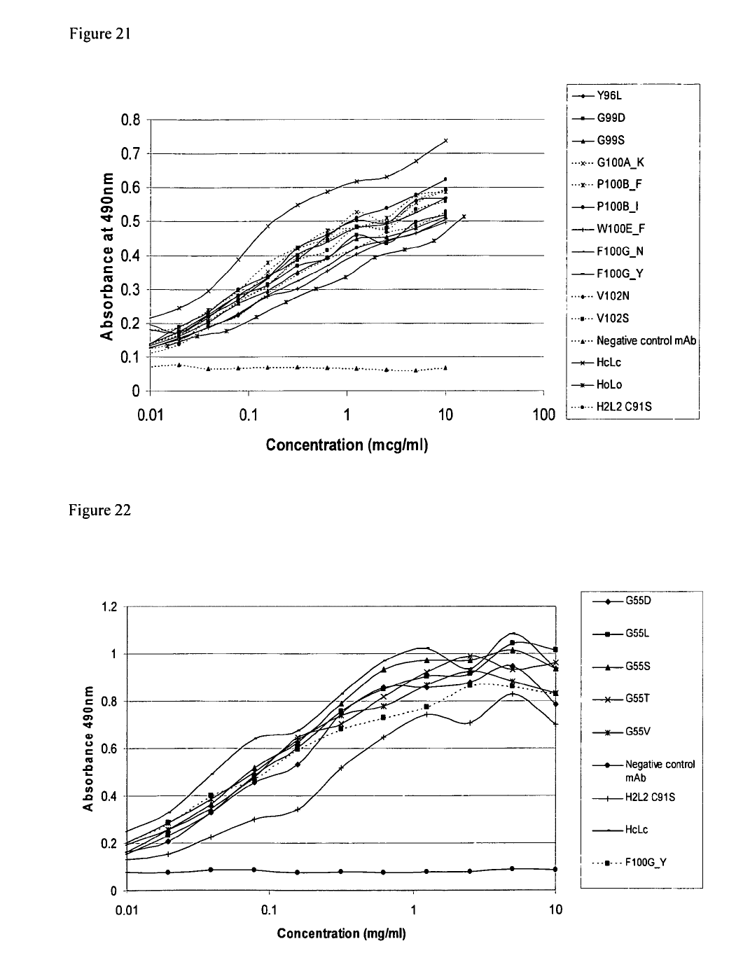

The experiment is illustrated in FIG. 21 where H2L2-C91S, H0L0, HcLc (10B3 chimera) and a negative control monoclonal antibody were used as control antibodies. All eleven CDRH3 variant antibodies bound to recombinant myostatin in this ELISA. H2L2-C91S_P100B_I, H2L2-C91S_V102N, H2L2-C91S_G100A_K, H2L2-C91S_P100B_F and H2L2-C91S_F100G_Y tended to have better binding activity for myostatin than base H2L2-C91S and H0L0.

### 6.8 Myostatin Competition ELISA

The CDRH3 variants were further investigated in three different myostatin competition ELISAs. The purified antibodies were analyzed for the ability to compete with the 10B3 murine mAb.

### 6.8.1 Using Polyclonal Ab as Capture Method

The protocol set out in 6.7 was used with the addition of 10B3 (final concentration of 0.3 m/ml) to each well and mixed with the antibodies titrated out to a suitable concentration range (approximately 10 to 0.01 μg/ml). An anti-mouse HRP-conjugated antibody (DAKO P0260, used according to the manufacturer's instructions) was used to detect binding of the 10B3 antibody. The ranking obtained from the ELISA data is shown in Table 19.

### 6.8.2 Using Biotinylated Myostatin as Capture Method

The protocol set out in 6.7 was used but the plates were initially coated at 4° C. overnight with 5 μg/ml of streptavidin. Biotinylated myostatin was added at 0.3 μg/ml block buffer during 1 hour followed by 3-times in wash buffer. 10B3 (final concentration of 0.2 μg/ml) was added into each well and mixed with antibodies titrated out to a suitable concentration range (approximately 10 to 0.01 μg/ml). An anti-mouse HRP-conjugated antibody (DAKO P0260, used according to the manufacturer's instructions) was used to detect binding of the 10B3 antibody. The ranking obtained from the ELISA data is shown in Table 19.

### 6.8.3 Using Myostatin as Capture Method (Direct Capture)

The protocol set out in 6.7 was used but the plates were initially coated at 4° C. overnight with 0.2 μg/ml of myostatin (recombinant in-house, see 1.1 above). 10B3 (final concentration of 0.3 μg/ml) was added into each well and mixed with antibodies titrated out to a suitable concentration range (approximately 10 to 0.01 μg/ml). An anti-mouse HRP-conjugated antibody (DAKO P0260, used according to the manufacturer's instructions) was used to detect binding of the 10B3 antibody. The ranking obtained from the ELISA data is shown in Table 19.

All the CDRH3 variants were able to compete against 10B3. The five most potent molecules from each of the different competition ELISAs are listed in Table 19.

TABLE 19
Ranking order top (1) to bottom (5) of
five most potent CDRH3 variant molecules
Myostatin competition ELISA

| Biotinylated myostatin | Polyclonal Abs | Direct capture |
|---|---|---|
| H2L2-C91S _V102S | H2L2-C91S _P100B_F | H2L2-C91S |
| | | _P100B_F |
| H2L2-C91S _F100G_Y | H2L2-C91S _V102N | H2L2-C91S |
| | | _F100G_Y |
| H2L2-C91S _P100B_I | H2L2-C91S _V102S | H2L2-C91S |
| | | _V102N |
| H2L2-C91S _V102N | H2L2-C91S _F100G_Y | H2L2-C91S |
| | | _V102S |
| H2L2-C91S _Y96L | H2L2-C91S _G99D | H2L2-C91S |
| | | _P100B_I |

On the basis of the analysis in this section (6.8) and the previous BIAcore data in 6.6 and 6.7, the variants H2L2-C91S_P100B_F, H2L2-C91S_P100B_I, H2L2-C91S_F100G_Y, H2L2-C91S_V102N and H2L2-C91S_V102S were selected for further analyses.

### 6.9 Inhibition of Biological Activity of Myostatin In Vitro

The five selected CDRH3 variants of 6.8 were tested in the myostatin responsive reporter gene assay (see 1.2 above), to assess in vitro efficacy. Myostatin at a concentration of 5 nM was pre-incubated with varying concentrations of antibody at 37° C. prior to addition to transfected A204 cells. The cells were incubated at 37° C. for a further 6 hours before relative luciferase expression was determined by luminescence. The resulting IC50s are shown in Table 20.

TABLE 20
IC50 of humanised antibodies in A204 in vitro activity assay

| | | Lower | Upper |
|---|---|---|---|
| | Mean IC50 | 95% CI | 95% CI |
| Antibody | (nM) | (nM) | (nM) |
| 10B3 Chimera | 3.534 | 1.941 | 6.435 |
| H2L2-C91S | 5.137 | 2.350 | 11.230 |
| H2L2-C91S _P100B_F | 4.235 | 2.295 | 7.818 |
| H2L2-C91S _P100B_I | 4.525 | 1.837 | 11.140 |
| H2L2-C91S _F100G_Y | 3.639 | 1.908 | 6.940 |
| H2L2-C91S _V102N | 5.514 | 3.023 | 10.060 |
| H2L2-C91S _V102S | 4.221 | 2.234 | 7.975 |

The data demonstrate that all the antibodies tested neutralised myostatin with a similar potency to the 10B3 chimera with H2L2-C91S_F100G_Y having the highest potency although not significantly so in this assay.

### 7. Construction and Expression of Fc Disabled Constant Region Variant

As the mode of action of anti-myostatin in vivo will be the simple binding and neutralisation of myostatin, it may not be necessary that the molecule retain its Fc-function to elicit ADCC and CDC responses. Furthermore, disabling Fc function may help mitigate against the potential for infusion-related immune reactions. The mutation to disable Fc function involves the following substitutions, using EU numbering system: Leu 235 Ala; and Gly 237 Ala.

Using standard molecular biology techniques, the gene encoding the sequence for the variable heavy region of the CDRH3 variant H2_F100G_Y was transferred from the existing construct to an expression vector containing the hIgG1 Fc disabled constant region. Full length DNA expression constructs encoding the heavy chain (SEQ ID NO: 98 H2_F100G_Y_Fc Disabled) and the light chain (SEQ ID NO: 40 L2-C91S) were produced using pTT vectors. Details of the heavy chain are in Table 21.

TABLE 21
Sequence of CDRH3 variant Fc disabled

| Name | Full length Protein Seq ID |
|---|---|
| H2L2-C91S _F100G_Y Fc Disabled | 98 |

The effect of the Fc disabled constant region was analyzed in the myostatin responsive reporter gene assay, (described above at 1.2). The resulting IC50 data are shown in Table 22.

TABLE 22
IC50 of CDRH3 variant Fc disabled antibody
in A204 in vitro activity assay

| | | Lower | Upper |
|---|---|---|---|
| | Mean IC50 | 95% CI | 95% CI |
| Antibody | (nM) | (nM) | (nM) |
| H2L2-C91S | 4.083 | 1.319 | 12.640 |
| H2L2-C91S _F100G_Y Fc Disabled | 1.239 | 0.524 | 2.932 |

These data demonstrate that disabling the Fc-function of “H2L2-C91S_F100G_Y_Fc Disabled” as described above has no significant effect on the antibody's potency to neutralise myostatin.

### 8. CDRH2 Variant Humanised Antibodies

### 8.1 Construction of CDRH2 Variant Humanised Antibodies

As described above at Example 5, the asparagine at Kabat position 54 (N54) in heavy chain CDRH2 has potential for deamidation. In order to mitigate this potential risk this amino acid was mutated to generate a number of CDRH2 variants of H2_F100G_Y. These all differed in CDRH2 (SEQ ID NO: 2) and were generated by site directed mutagenesis using the pTT vector coding for the H2_F100G_Y heavy chain. The light chain (SEQ ID NO: 40 L2-C91S) was expressed with each of the heavy chains. These constructs were not disabled in the Fc region.

### 8.2 CDRH2 Variant Expression in HEK293 6E Cells

The pTT plasmids encoding the heavy and light chains respectively were transiently co-transfection in HEK 293 6E cells as described above at 6.2. In addition H2L2-C91S_F100G_Y was expressed as a positive control. Antibodies produced in the HEK293 cell supernatant were analyzed for binding to recombinant myostatin by BIAcore. The screen of the CDRH2 variants indicated that all bind to recombinant myostatin.

Using the affinity data obtained and the in silico analysis for potential deamidation risk, a panel of five CDRH2 variants (listed in Table 23) were selected for larger scale expression, purification and further analysis.

TABLE 23
CDRH2 variant sequences

| Name | Sequence of CDRH2 |
|---|---|
| H2L2 C91S | NIYPYNGVSNYNQRFKA |
| | (SEQ ID NO: 2) |
| H2L2 C91S_G55D F100G_Y | NIYPYNDVSNYNQRFKA |
| | (SEQ ID NO: 93) |
| H2L2 C91S_G55L F100G_Y | NIYPYNLVSNYNQRFKA |
| | (SEQ ID NO: 94) |
| H2L2 C91S_G55S F100G_Y | NIYPYNSVSNYNQRFKA |
| | (SEQ ID NO: 95) |
| H2L2 C91S_G55T F100G_Y | NIYPYNTVSNYNQRFKA |
| | (SEQ ID NO: 96) |
| H2L2 C91S_G55V F100G_Y | NIYPYNVVSNYNQRFKA |
| | (SEQ ID NO: 97) |

### 8.3 Characterization of CDRH2 Variants

All five antibodies were analyzed for binding activity in the myostatin binding ELISA (as described in example 4.2). FIG. 22 shows the results for H2L2-C91S_F100G_Y, H2L2 C91S, HcLc (10B3C) and a negative control mAb; and all five CDRH2 variant antibodies. The CDRH2 variants had better or similar binding activity for myostatin as H2L2-C91S_F100G_Y.

### 8.4 CDRH2 Variant BIAcore Analysis

The CDRH2 variants were also tested to determine any changes in myostatin binding affinity by BIAcore. Protein A was immobilised on a C1 Biacore biosensor chip, purified antibodies were captured at a low density so that maximal binding of myostatin resulted in less than 30 resonance units. Myostatin was passed over the captured antibody surface at a concentration of 256 nM only; a buffer injection (i.e. 0 nM) was used to double reference the binding data. Regeneration of the Protein A surface was using 100 mM phosphoric acid. Data was fitted to the Bivalent model and to the Two State Model, both inherent to the T100 Biacore analysis software. However since myostatin is a dimer more weight was given to the Bivalent model data. The run was carried out using HBS-EP and at a temperature of 25° C.

The models used may not reflect the true binding in vivo and the models themselves may not accurately reflect the interaction, so the calculated values were for ranking only. The data suggests that compared to H2L2-C91S_F100G_Y, the CDRH2 variants do not impact too significantly on affinity, with the worst construct by the Bivalent model (H2L2 C91S_G55L F100G_Y) showing a 6.8 fold worsening of overall affinity.

### 8.5 Inhibition of Biological Activity of Myostatin In Vitro

The effect of the CDRH2 variants on in vitro neutralisation assays was also undertaken using the A204 luciferase assay (described in section 1.2). The IC50 values of the inhibition curves are presented in Table 24.

TABLE 24
IC50 of antibody variants in A204 in vitro activity assay

| | | Lower | Upper |
|---|---|---|---|
| | Mean IC50 | 95% CI | 95% CI |
| Antibody | (nM) | (nM) | (nM) |
| 10B3 Chimera | 3.570 | 1.473 | 8.654 |
| H2L2-C91S _F100G_Y | 11.070 | 3.686 | 33.230 |
| H2L2 C91S _G55D F100G_Y | 5.530 | 1.649 | 18.540 |
| H2L2 C91S _G55L F100G_Y | 5.581 | 1.601 | 19.460 |
| H2L2 C91S _G55S F100G_Y | 4.425 | 1.730 | 11.310 |
| H2L2 C91S _G55T F100G_Y | 6.892 | 2.452 | 19.370 |
| H2L2 C91S _G55V F100G_Y | 3.840 | 1.044 | 14.130 |

The data indicate that all the CDRH2 variant antibodies inhibit myostatin-induced activation of A204 cells with a similar potency to H2L2-C91S_F100G_Y in this assay.

### 8.6 Fc-Disabled CDRH2 Variant

H2L2 C91S_G55S F100G_Y, the developability enhanced molecule with the highest apparent potency in the A204 assay was Fc-disabled (by making the following substitutions, using EU numbering system: Leu 235 Ala; and Gly 237 Ala) as exemplified in SEQ ID NO: 99. The receptor binding assay (Example 2.5) was used to demonstrate that this new molecule H2L2 C91S_G55S F100G_Y-Fc disabled had slightly improved potency relative to H2L2 C91S_G55S F100G_Y (see Table 25).

TABLE 25
IC50 values of antibody variants in ActRIIb receptor binding assay

| | | Lower | Upper |
|---|---|---|---|
| | Mean IC50 | 95% CI | 95% CI |
| mAb | (nM) | (nM) | (nM) |
| 10B3 | 0.161 | 0.087 | 0.295 |
| H2L2 C91S_G55S F100G_Y | 0.786 | 0.326 | 1.898 |
| H2L2 C91S_G55S F100G_Y-Fc | 0.518 | 0.206 | 1.298 |
| disabled | | | |

### 9. Efficacy of 10B3 in Glucocorticoid-Induced Muscle Wasting

In the present study, we investigated whether 10B3 treatment could prevent steroid induced muscle loss in mice. C57BL mice were treated with PBS, mIgG2a or 10B3. Dexamethasone was used as the steroid to induce muscle loss

Dexamethasone treatment caused body weight loss in animals pre-treated with the control antibody. The dexamethasone-induced weight loss was attenuated by pre-treatment with 10B3. Animals pre-treated with the control antibody showed muscle atrophy in extensor digitorum longus (EDL), tibialis anterior (TA), and gastrocnemius. In contrast, dexamethasone treatment in animals pre-treated with 10B3 did not cause atrophy in TA, EDL, and gastrocnemius. Animals pre-treated with the control antibody showed an increase in body fat accumulation. However, there was no increase in % body fat after dexamethasone treatment in animals pre-treated with 10B3.

These results in Example 9 suggest that 10B3 or the humanised antibody thereof may be used for treatment of glucocorticoids-induced muscle wasting. For example, prophylactic treatment of muscle wasting in patients on glucocorticoid therapy may be advantageous.

### 10. 10B3 Treatment Attenuated Muscle Atrophy in Sciatic Nerve Crush Model

Here we used the nerve injury model to evaluate the efficacy of 10B3 in prevention of disuse atrophy in mice.

C57BL mice were treated with mIgG2a control or 10B3 antibody. The right sciatic nerve in the mid thigh was exposed and either left intact (sham group) or injured by crushing for 10 seconds using a haemostatic forceps (nerve crush group). Sciatic nerve crush injury resulted in decreases in the mass of extensor digitorum longus (EDL), tibialis anterior (TA), gastrocnemius and soleus as compared to the sham control. In sham surgery groups, 10B3 treatment increased the mass of TA, EDL, gastrocnemius and quadriceps when compared to IgG2a control group. Animals treated with 10B3 retained more muscle than IgG2a control treated animals. 10B3 treatment also increased total body weight in both sham-operated and nerve crushed animals.

These results demonstrate that 10B3 or the humanised antibody thereof may have the potential for prevention and/or treatment of human disuse muscle atrophy.

### 11. 10B3 Treatment Attenuated Muscle Wasting in C-26 Tumour-Bearing Mice

In the current study, the effect of 10B3 treatment on body weight change, muscle mass and function were studies in Colon-26 tumour bearing mice, a widely used preclinical model for cancer cachexia studies.

Thirty eight 8-week-old male CD2F1 mice were randomly divided into 4 groups: mIgG2a (n=9) 10B3 (n=9), mIgG2a+C-26 (n=10), and 10B3+C-26 (n=10). Colon-26 (C-26) tumour cells were subcutaneously implanted into 20 mice at 1×10⁶ cells/mouse. Several hours later, animals began to receive antibody injections. Mice were injected i.p. with either mouse IgG2a control antibody or 10B3 at the dose of 30 mg/kg on day 0, 3, 7, 14, 21. Body weight and fat mass were monitored throughout the experiment. Shortly before sacrifice on day 25, lower limb muscle strength was assessed by measuring the contraction force upon the electrical stimulation of sciatic nerve in the mid thigh. The tumour weight, and individual muscle mass and epididymal fat pad mass were determined at the end of the experiment.

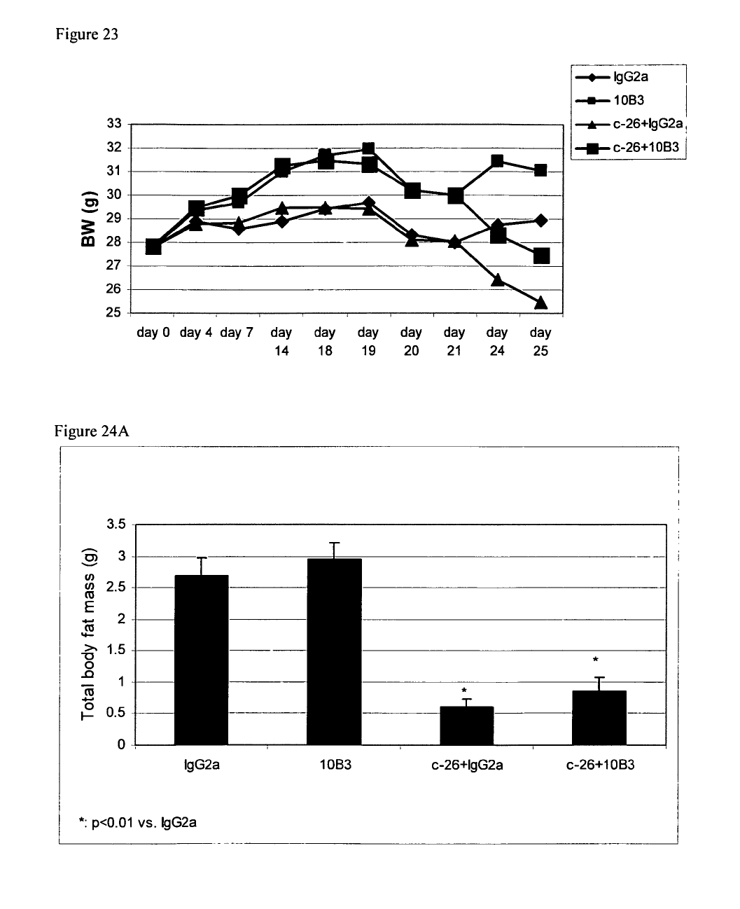

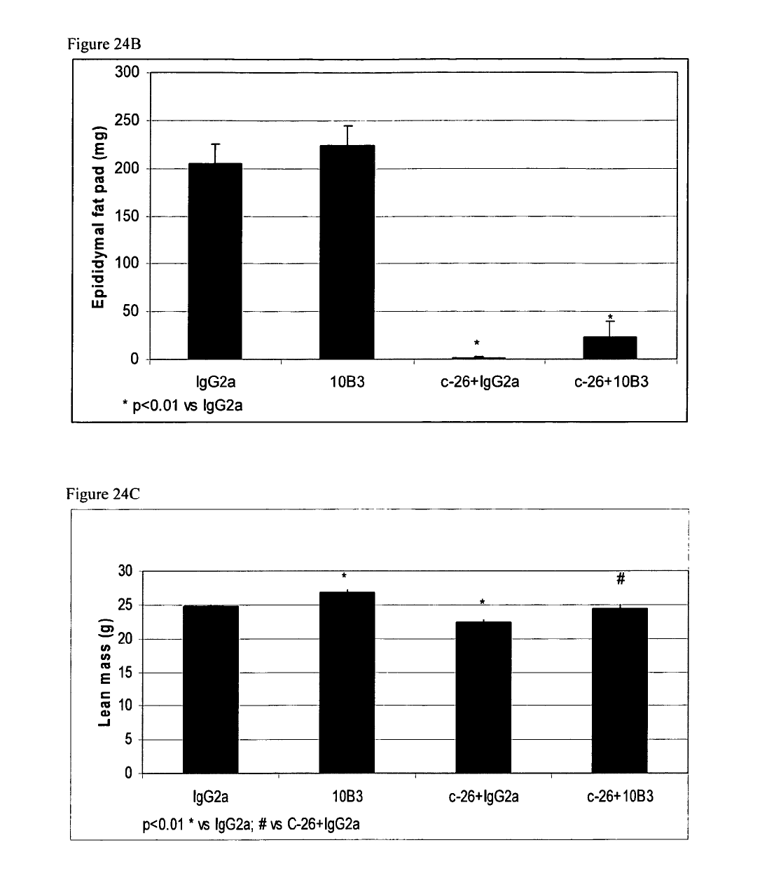

FIG. 23 shows the effect of antibody treatment on body weight in C-26 tumour bearing mice from day 0 to day 25. Tumour bearing mice start to lose body weight dramatically at 21 days after tumour implantation. Treatment with 10B3 effectively mitigated weight loss in tumour bearing mice. The average body weight of the tumour bearing mice treated with 10B3 was 8% higher than that of tumour bearing mice treated with mIgGa2a control antibody. There was no significant difference in tumour size (2.2 g for IgG2a vs 1.9 g for 10B3) between 10B3 treated and mIgG2a control treated groups.

FIG. 24 shows the effect of antibody treatment on total body fat (A), epididymal fat pad (B) and lean mass (C) in C-26 tumour bearing mice. Tumour bearing mice had significantly less total body fat (FIG. 24A). Epididymal fat pad almost completely disappeared in both 10B3 and mIgG2a control treated tumour bearing mice (FIG. 24B), suggesting that 10B3 does not protect tumour bearing animals against body fat loss.

As shown in FIG. 24C, 10B3 treatment causes significant (p<0.01) increase in lean mass in both normal animals as well as tumour bearing mice. Tumour bearing mice treated with control IgG2a had significantly lower lean mass after tumour removal. In contrast, tumour bearing mice treated with 10B3 had significantly (p<0.01) greater lean mass than IgG2a treated tumour bearing mice. In fact, there was no significant difference in lean mass between 10B3 treated tumour bearing mice and normal animals.

Table 26 shows the effect of antibody treatment on muscle mass. As expected, tumour bearing mice had significant loss of TA, EDL, quadriceps, soleus and gastrocnemius muscle (Table 26). 10B3 treatment increased muscle mass in normal animals. Most importantly, 10B3 treatment attenuated muscle loss in tumor bearing mice. In tumour bearing mice treated with 10B3, the weights of TA, EDL, quadriceps, soleus and gastrocnemius muscles were 17.8%, 11.3%, 16.9%, 13.4% and 14.6% greater than those of tumour bearing mice treated with IgG2a control, respectively.

TABLE 26
10B3 treatment attenuated muscle loss in tumor bearing mice.

| Groups | quadriceps | gastrocnemius | TA | EDL | soleus |
|---|---|---|---|---|---|
| IgG2a | 216 +/− 2.1 | 159 +/− 2.2 | 51 +/− 0.5 | 11.1+/0.5 | 8.0 +/− 0.4 |
| 10B3 | 244 +/− 4.7 * | 173 +/− 4.8 | 58 +/− 1.2 * | 12.6+/0.6 * | 8.5 +/− 0.2 |
| C-26 + IgG2a | 174 +/− 3.7 * | 123 +/− 4.5 * | 40 +/− 1.6 * | 8.9 +/− 0.3 * | 6.9 +/− 0.3 * |
| C-26 + 10B3 | 204 +/− 8.6 ^{#} | 140 +/− 5.8 * | 47 +/− 1.8 ^{#} | 9.9 +/− 0.6 ^{#} | 7.9 +/− 0.5 ^{#} |

Data are mean muscle mass (mg) +/− SEM. The means with the superscripts * and ^{#} indicates significantly (p < 0.05) different from IgG2a group and C-26 + IgG2a group, respectively according to Student T tests.

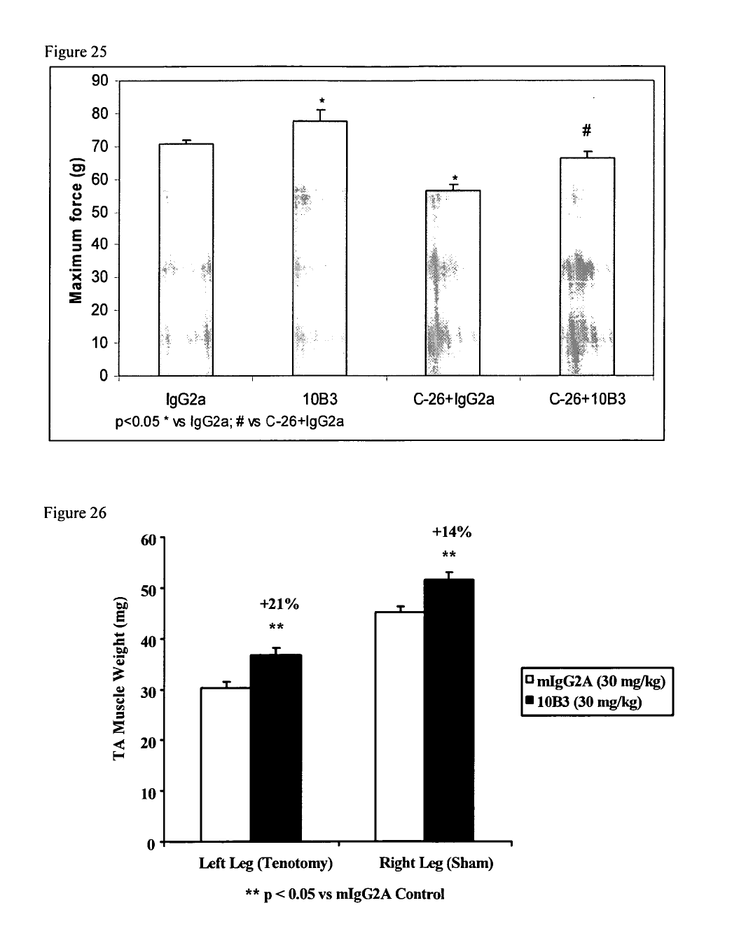

FIG. 25 shows the effect of antibody treatment on lower limb muscle strength, which was assessed by measuring the contraction force upon the electrical stimulation of sciatic nerve in the mid thigh. After 25 days of tumour implant, lower limb muscle contraction force was significantly (p<0.001) reduced by 20% in the control antibody groups. 10B3 treatment increased maximum contraction force by 10.2% and 17.5% in healthy animals and tumour bearing mice, respectively, as compared to the control groups (p<0.05). There was no significant difference in maximum force measurement between 10B3 treated tumour bearing mice and healthy controls. Thus, 10B3 treatment improved muscle function in both healthy and tumour bearing mice.

These data indicate that 10B3 or the humanised antibody thereof treatment could attenuate muscle loss and functional decline associated with cancer cachexia.

### 12. Effects of 10B3 Treatment on Skeletal Muscle Atrophy in Mouse Tenotomy Model

Here, we determined the effects of the myostatin antibody 10B3 on muscle mass in a mouse tenotomy model.

Young adult male C57BL mice were randomly divided into mIgG2a or 10B3 treatment groups (n=6/group) and dosed i.p. at 30 mg/kg on day 1, 4, 8, and 15. On the morning prior to dosing (day 0), all mice received the following surgical protocol: tibialis anterior (TA) tendons were separated at their distal insertion in left legs (tenotomy) while all right TA tendons were exposed but left intact (sham). After 3 weeks (day 21), mice were euthanized to assess changes in TA muscle mass.

Three-week treatment of 10B3 significantly increased TA muscle mass following both sham and tenotomy surgeries in mice (FIG. 26). Interestingly, the effect of 10B3 was more pronounced in the presence of tenotomy (+21%) compared to the intact sham condition (+14%).

These data indicate that 10B3 or the humanised antibody thereof treatment could attenuate muscle loss and functional decline associated with trauma/injury.

SEQUENCES

(CDRH1)
SEQ ID NO: 1
GYFMH
(CDRH2)
SEQ ID NO: 2
NIYPYNGVSNYNQRFKA
(CDRH3)
SEQ ID NO: 3
RYYYGTGPADWYFDV
(CDRL1)
SEQ ID NO: 4
KASQDINSYLS
(CDRL2)
SEQ ID NO: 5
RANRLVD
(CDRL3)
SEQ ID NO: 6
LQCDEFPLT
(mouse monoclonal 10B3 V_{H})
SEQ ID NO: 7
EVQLQQSGPELVKPGASVKISCKASGYSFTGYFMHWVKQSHGNILDW
IGNIYPYNGVSNYNQRFKAKATLTVDKSSSTAYMELRSLTSEDSAVY
YCARRYYYGTGPADWYFDVWGTGTTVTVSS
(mouse monoclonal 10B3 and 10B3 chimera V_{L})
SEQ ID NO: 8
DIKMTQSPSSMYASLRERVTITCKASQDINSYLSWFQQKPGKSPKTL
IYRANRLVDGVPSRFSGSGSGQDYSLTISSLEYEDMGIYYCLQCDEF
PLTFGAGTKLELK
(artificial signal sequence)
SEQ ID NO: 9
MGWSCIILFLVATATGVHS
(human acceptor framework for V_{H})
SEQ ID NO: 10
QVQLVQSGAEVKKPGASVKVSCKASGYTFTSYGISWVRQAPGQGLEW
MGWISAYNGNTNYAQKLQGRVTMTTDTSTSTAYMELRSLRSDDTAVY
YCARXXXXXXXXXXWGQGTMVTVSS
(human acceptor framework for V_{L})
SEQ ID NO: 11
DIQMTQSPSSLSASVGDRVTITCRASQGISNYLAWFQQKPGKAPKSL
IYAASSLQSGVPSKFSGSGSGTDFTLTISSLQPEDFATYYCXXXXXX
XXXXFGQGTKLEIK
(humanised V_{H}: H0)
SEQ ID NO: 12
QVQLVQSGAEVKKPGASVKVSCKASGYTFTGYFMHWVRQAPGQGLEW
MGNIYPYNGVSNYNQRFKARVTMTTDTSTSTAYMELRSLRSDDTAVY
YCARRYYYGTGPADWYFDVWGQGTLVTVSS
(humanised V_{H}: H1)
SEQ ID NO: 13
QVQLVQSGAEVKKPGASVKVSCKASGYTFTGYFMHWVRQAPGQGLEW
MGNIYPYNGVSNYNQRFKARVTMTTDTSTSTAYMELRSLRSDDTAVY
YCARRYYYGTGPADWYFDVWGTGTLVTVSS
(humanised V_{H}: H2)
SEQ ID NO: 14
QVQLVQSGAEVKKPGASVKVSCKASGYSFTGYFMHWVRQAPGQGLEW
MGNIYPYNGVSNYNQRFKARVTMTTDTSTSTAYMELRSLRSDDTAVY
YCARRYYYGTGPADWYFDVWGQGTLVTVSS
(humanised V_{L}: L0)
SEQ ID NO: 15
DIQMTQSPSSLSASVGDRVTITCKASQDINSYLSWFQQKPGKAPKSL
IYRANRLVDGVPSKFSGSGSGTDFTLTISSLQPEDFATYYCLQCDEF
PLTFGQGTKLEIK
(humanised V_{L}: L1)
SEQ ID NO: 16
DIQMTQSPSSLSASVRDRVTITCKASQDINSYLSWFQQKPGKAPKSL
IYRANRLVDGVPSKFSGSGSGTDFTLTISSLQPEDFATYYCLQCDEF
PLTFGQGTKLEIK
(humanised V_{L}: L2)
SEQ ID NO: 17
DIQMTQSPSSLSASVGDRVTITCKASQDINSYLSWFQQKPGKAPKSL
IYRANRLVDGVPSKFSGSGSGTDYTLTISSLQPEDFATYYCLQCDEF
PLTFGQGTKLEIK
(humanised V_{L}: L3)
SEQ ID NO: 18
DIQMTQSPSSLSASVGDRVTITCKASQDINSYLSWFQQKPGKAPKSL
IYRANRLVDGVPSKFSGSGSGTDFTLTISSLQPEDFATYYCLQCDEF
PLTFGAGTKLEIK
(10B3 chimera V_{H}: N54D)
SEQ ID NO: 19
EVQLQQSGPELVKPGASVKISCKASGYSFTGYFMHWVKQSHGNILDW
IGNIYPYDGVSNYNQRFKAKATLTVDKSSSTAYMELRSLTSEDSAVY
YCARRYYYGTGPADWYFDVWGTGTLVTVSS
(10B3 chimera V_{H}: N54Q)
SEQ ID NO: 20
EVQLQQSGPELVKPGASVKISCKASGYSFTGYFMHWVKQSHGNILDW
IGNIYPYQGVSNYNQRFKAKATLTVDKSSSTAYMELRSLTSEDSAVY
YCARRYYYGTGPADWYFDVWGTGTLVTVSS
(10B3 chimera V_{L}: C91S)
SEQ ID NO: 21
DIKMTQSPSSMYASLRERVTITCKASQDINSYLSWFQQKPGKSPKTL
IYRANRLVDGVPSRFSGSGSGQDYSLTISSLEYEDMGIYYCLQSDEF
PLTFGAGTKLELK
(humanised V_{H}: H2: N54D)
SEQ ID NO: 22
QVQLVQSGAEVKKPGASVKVSCKASGYSFTGYFMHWVRQAPGQGLEW
MGNIYPYDGVSNYNQRFKARVTMTTDTSTSTAYMELRSLRSDDTAVY
YCARRYYYGTGPADWYFDVWGQGTLVTVSS
(humanised V_{H}: H2: N54Q)
SEQ ID NO: 23
QVQLVQSGAEVKKPGASVKVSCKASGYSFTGYFMHWVRQAPGQGLEW
MGNIYPYQGVSNYNQRFKARVTMTTDTSTSTAYMELRSLRSDDTAVY
YCARRYYYGTGPADWYFDVWGQGTLVTVSS
(humanised V_{L}: L2: C91S)
SEQ ID NO: 24
DIQMTQSPSSLSASVGDRVTITCKASQDINSYLSWFQQKPGKAPKSL
IYRANRLVDGVPSKFSGSGSGTDYTLTISSLQPEDFATYYCLQSDEF
PLTFGQGTKLEIK
(10B3 chimera V_{H})
SEQ ID NO: 25
EVQLQQSGPELVKPGASVKISCKASGYSFTGYFMHWVKQSHGNILDW
IGNIYPYNGVSNYNQRFKAKATLTVDKSSSTAYMELRSLTSEDSAVY
YCARRYYYGTGPADWYFDVWGTGTLVTVSS
(10B3 chimera heavy chain)
SEQ ID NO: 26
EVQLQQSGPELVKPGASVKISCKASGYSFTGYFMHWVKQSHGNILDW
IGNIYPYNGVSNYNQRFKAKATLTVDKSSSTAYMELRSLTSEDSAVY
YCARRYYYGTGPADWYFDVWGTGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(10B3 chimera light chain)
SEQ ID NO: 27
DIKMTQSPSSMYASLRERVTITCKASQDINSYLSWFQQKPGKSPKTL
IYRANRLVDGVPSRFSGSGSGQDYSLTISSLEYEDMGIYYCLQCDEF
PLTFGAGTKLELKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(humanised heavy chain: H0)
SEQ ID NO: 28
QVQLVQSGAEVKKPGASVKVSCKASGYTFTGYFMHWVRQAPGQGLEW
MGNIYPYNGVSNYNQRFKARVTMTTDTSTSTAYMELRSLRSDDTAVY
YCARRYYYGTGPADWYFDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised heavy chain: H1)
SEQ ID NO: 29
QVQLVQSGAEVKKPGASVKVSCKASGYTFTGYFMHWVRQAPGQGLEW
MGNIYPYNGVSNYNQRFKARVTMTTDTSTSTAYMELRSLRSDDTAVY
YCARRYYYGTGPADWYFDVWGTGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised heavy chain: H2)
SEQ ID NO: 30
QVQLVQSGAEVKKPGASVKVSCKASGYSFTGYFMHWVRQAPGQGLEW
MGNIYPYNGVSNYNQRFKARVTMTTDTSTSTAYMELRSLRSDDTAVY
YCARRYYYGTGPADWYFDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised light chain: L0)
SEQ ID NO: 31
DIQMTQSPSSLSASVGDRVTITCKASQDINSYLSWFQQKPGKAPKSL
IYRANRLVDGVPSKFSGSGSGTDFTLTISSLQPEDFATYYCLQCDEF
PLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(humanised light chain: L1)
SEQ ID NO: 32
DIQMTQSPSSLSASVRDRVTITCKASQDINSYLSWFQQKPGKAPKSL
IYRANRLVDGVPSKFSGSGSGTDFTLTISSLQPEDFATYYCLQCDEF
PLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(humanised light chain: L2)
SEQ ID NO: 33
DIQMTQSPSSLSASVGDRVTITCKASQDINSYLSWFQQKPGKAPKSL
IYRANRLVDGVPSKFSGSGSGTDYTLTISSLQPEDFATYYCLQCDEF
PLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(humanised light chain: L3)
SEQ ID NO: 34
DIQMTQSPSSLSASVGDRVTITCKASQDINSYLSWFQQKPGKAPKSL
IYRANRLVDGVPSKFSGSGSGTDFTLTISSLQPEDFATYYCLQCDEF
PLTFGAGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(10B3 chimera N54D heavy chain)
SEQ ID NO: 35
EVQLQQSGPELVKPGASVKISCKASGYSFTGYFMHWVKQSHGNILDW
IGNIYPYDGVSNYNQRFKAKATLTVDKSSSTAYMELRSLTSEDSAVY
YCARRYYYGTGPADWYFDVWGTGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(10B3 chimera N54Q heavy chain)
SEQ ID NO: 36
EVQLQQSGPELVKPGASVKISCKASGYSFTGYFMHWVKQSHGNILDW
IGNIYPYQGVSNYNQRFKAKATLTVDKSSSTAYMELRSLTSEDSAVY
YCARRYYYGTGPADWYFDVWGTGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(10B3 chimera C91S light chain)
SEQ ID NO: 37
DIKMTQSPSSMYASLRERVTITCKASQDINSYLSWFQQKPGKSPKTL
IYRANRLVDGVPSRFSGSGSGQDYSLTISSLEYEDMGIYYCLQSDEF
PLTFGAGTKLELKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(humanised heavy chain: H2 N54D)
SEQ ID NO: 38
QVQLVQSGAEVKKPGASVKVSCKASGYSFTGYFMHWVRQAPGQGLEW
MGNIYPYDGVSNYNQRFKARVTMTTDTSTSTAYMELRSLRSDDTAVY
YCARRYYYGTGPADWYFDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised heavy chain: H2 N54Q)
SEQ ID NO: 39
QVQLVQSGAEVKKPGASVKVSCKASGYSFTGYFMHWVRQAPGQGLEW
MGNIYPYQGVSNYNQRFKARVTMTTDTSTSTAYMELRSLRSDDTAVY
YCARRYYYGTGPADWYFDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised light chain: L2 C91S)
SEQ ID NO: 40
DIQMTQSPSSLSASVGDRVTITCKASQDINSYLSWFQQKPGKAPKSL
IYRANRLVDGVPSKFSGSGSGTDYTLTISSLQPEDFATYYCLQSDEF
PLTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
(10B3 chimera heavy chain, DNA sequence)
SEQ ID NO: 41
ATGGGATGGAGCTGTATCATCCTCTTCTTGGTAGCAACAGCTACAGG
TGTCCACTCCGAGGTTCAGCTGCAGCAGTCTGGACCTGAACTGGTGA
AGCCTGGGGCTTCAGTGAAGATATCCTGCAAGGCTTCTGGTTACTCA
TTCACTGGCTACTTCATGCACTGGGTGAAGCAGAGCCATGGCAATAT
CCTCGATTGGATTGGAAATATTTATCCTTACAATGGTGTTTCTAACT
ACAACCAGAGATTCAAGGCCAAGGCCACATTGACTGTAGACAAGTCC
TCTAGTACAGCCTACATGGAGCTCCGCAGCCTTACATCTGAGGACTC
TGCAGTCTATTACTGTGCAAGACGCTATTACTACGGTACCGGACCGG
CTGATTGGTACTTCGATGTCTGGGGCACTGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(10B3 chimera light chain, DNA sequence)
SEQ ID NO: 42
ATGGGATGGAGCTGTATCATCCTCTTCTTGGTAGCAACAGCTACAGG
TGTCCACTCCGACATCAAGATGACCCAGTCTCCATCTTCCATGTATG
CATCTCTACGAGAGAGAGTCACTATCACTTGCAAGGCGAGTCAGGAC
ATTAATAGCTATTTAAGCTGGTTCCAGCAGAAACCAGGGAAATCTCC
TAAGACCCTAATCTATCGTGCAAACAGATTGGTAGATGGGGTCCCAT
CAAGGTTCAGTGGCAGTGGATCTGGGCAAGATTATTCTCTCACCATC
AGCAGCCTGGAGTATGAAGATATGGGAATTTATTATTGTCTACAGTG
TGATGAATTTCCGCTCACGTTCGGTGCTGGGACCAAGCTGGAGCTGA
AACGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(humanised heavy chain: H0, DNA sequence)
SEQ ID NO: 43
ATGGGCTGGTCCTGCATCATCCTGTTTCTGGTGGCCACCGCCACCGG
CGTGCACAGCCAGGTGCAGCTGGTGCAGAGCGGCGCAGAGGTGAAGA
AGCCCGGCGCCAGCGTGAAAGTGAGCTGCAAGGCCAGCGGCTACACC
TTCACCGGCTACTTCATGCACTGGGTGAGGCAGGCTCCCGGCCAGGG
CCTGGAGTGGATGGGCAACATCTACCCCTACAACGGCGTCAGCAACT
ACAACCAGAGGTTCAAGGCCAGGGTGACCATGACCACCGACACCTCT
ACCAGCACCGCCTACATGGAACTGAGGAGCCTGAGGAGCGACGACAC
CGCCGTGTACTACTGCGCCAGGAGGTACTATTACGGCACCGGACCCG
CCGATTGGTACTTCGACGTGTGGGGACAGGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(humanised heavy chain: H1, DNA sequence)
SEQ ID NO: 44
ATGGGCTGGTCCTGCATCATCCTGTTTCTGGTGGCCACCGCCACCGG
CGTGCACAGCCAGGTGCAGCTGGTGCAGAGCGGCGCAGAGGTGAAGA
AGCCCGGCGCCAGCGTGAAAGTGAGCTGCAAGGCCAGCGGCTACACC
TTCACCGGCTACTTCATGCACTGGGTGAGGCAGGCTCCCGGCCAGGG
CCTGGAGTGGATGGGCAACATCTACCCCTACAACGGCGTCAGCAACT
ACAACCAGAGGTTCAAGGCCAGGGTGACCATGACCACCGACACCTCT
ACCAGCACCGCCTACATGGAACTGAGGAGCCTGAGGAGCGACGACAC
CGCCGTGTACTACTGCGCCAGGAGGTACTATTACGGCACCGGACCCG
CCGATTGGTACTTCGACGTGTGGGGAACGGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(humanised heavy chain: H2, DNA sequence)
SEQ ID NO: 45
ATGGGCTGGTCCTGCATCATCCTGTTTCTGGTGGCCACCGCCACCGG
CGTGCACAGCCAGGTGCAGCTGGTGCAGAGCGGCGCAGAGGTGAAGA
AGCCCGGCGCCAGCGTGAAAGTGAGCTGCAAGGCCAGCGGCTACTCC
TTCACCGGCTACTTCATGCACTGGGTGAGGCAGGCTCCCGGCCAGGG
CCTGGAGTGGATGGGCAACATCTACCCCTACAACGGCGTCAGCAACT
ACAACCAGAGGTTCAAGGCCAGGGTGACCATGACCACCGACACCTCT
ACCAGCACCGCCTACATGGAACTGAGGAGCCTGAGGAGCGACGACAC
CGCCGTGTACTACTGCGCCAGGAGGTACTATTACGGCACCGGACCCG
CCGATTGGTACTTCGACGTGTGGGGACAGGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(humanised light chain: L0, DNA sequence)
SEQ ID NO: 46
ATGGGCTGGTCCTGCATCATCCTGTTTCTGGTGGCCACCGCCACCGG
CGTGCACAGCGACATTCAGATGACCCAGAGCCCCAGCTCTCTGAGCG
CCAGCGTGGGCGATAGGGTGACCATCACCTGCAAGGCCAGCCAGGAC
ATCAACAGCTACCTGAGCTGGTTCCAGCAGAAGCCCGGCAAGGCTCC
CAAGAGCCTGATCTACAGGGCCAACAGGCTCGTGGACGGCGTGCCTA
GCAAGTTTAGCGGCAGCGGAAGCGGCACAGACTTCACCCTGACCATC
AGCTCCCTGCAGCCCGAGGACTTCGCCACCTACTACTGCCTGCAGTG
CGACGAGTTCCCCCTGACCTTCGGCCAGGGCACCAAACTGGAGATCA
AGCGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(humanised light chain: L1, DNA sequence)
SEQ ID NO: 47
ATGGGCTGGTCCTGCATCATCCTGTTTCTGGTGGCCACCGCCACCGG
CGTGCACAGCGACATTCAGATGACCCAGAGCCCCAGCTCTCTGAGCG
CCAGCGTGCGCGATAGGGTGACCATCACCTGCAAGGCCAGCCAGGAC
ATCAACAGCTACCTGAGCTGGTTCCAGCAGAAGCCCGGCAAGGCTCC
CAAGAGCCTGATCTACAGGGCCAACAGGCTCGTGGACGGCGTGCCTA
GCAAGTTTAGCGGCAGCGGAAGCGGCACAGACTTCACCCTGACCATC
AGCTCCCTGCAGCCCGAGGACTTCGCCACCTACTACTGCCTGCAGTG
CGACGAGTTCCCCCTGACCTTCGGCCAGGGCACCAAACTGGAGATCA
AGCGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(humanised light chain: L2, DNA sequence)
SEQ ID NO: 48
ATGGGCTGGTCCTGCATCATCCTGTTTCTGGTGGCCACCGCCACCGG
CGTGCACAGCGACATTCAGATGACCCAGAGCCCCAGCTCTCTGAGCG
CCAGCGTGGGCGATAGGGTGACCATCACCTGCAAGGCCAGCCAGGAC
ATCAACAGCTACCTGAGCTGGTTCCAGCAGAAGCCCGGCAAGGCTCC
CAAGAGCCTGATCTACAGGGCCAACAGGCTCGTGGACGGCGTGCCTA
GCAAGTTTAGCGGCAGCGGAAGCGGCACAGACTACACCCTGACCATC
AGCTCCCTGCAGCCCGAGGACTTCGCCACCTACTACTGCCTGCAGTG
CGACGAGTTCCCCCTGACCTTCGGCCAGGGCACCAAACTGGAGATCA
AGCGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(humanised light chain: L3, DNA sequence)
SEQ ID NO: 49
ATGGGCTGGTCCTGCATCATCCTGTTTCTGGTGGCCACCGCCACCGG
CGTGCACAGCGACATTCAGATGACCCAGAGCCCCAGCTCTCTGAGCG
CCAGCGTGGGCGATAGGGTGACCATCACCTGCAAGGCCAGCCAGGAC
ATCAACAGCTACCTGAGCTGGTTCCAGCAGAAGCCCGGCAAGGCTCC
CAAGAGCCTGATCTACAGGGCCAACAGGCTCGTGGACGGCGTGCCTA
GCAAGTTTAGCGGCAGCGGAAGCGGCACAGACTTCACCCTGACCATC
AGCTCCCTGCAGCCCGAGGACTTCGCCACCTACTACTGCCTGCAGTG
CGACGAGTTCCCCCTGACCTTCGGCGCGGGCACCAAACTGGAGATCA
AGCGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(10B3 chimera N54D heavy chain, DNA sequence)
SEQ ID NO: 50
ATGGGATGGAGCTGTATCATCCTCTTCTTGGTAGCAACAGCTACAGG
TGTCCACTCCGAGGTTCAGCTGCAGCAGTCTGGACCTGAACTGGTGA
AGCCTGGGGCTTCAGTGAAGATATCCTGCAAGGCTTCTGGTTACTCA
TTCACTGGCTACTTCATGCACTGGGTGAAGCAGAGCCATGGCAATAT
CCTCGATTGGATTGGAAATATTTATCCTTACGATGGTGTTTCTAACT
ACAACCAGAGATTCAAGGCCAAGGCCACATTGACTGTAGACAAGTCC
TCTAGTACAGCCTACATGGAGCTCCGCAGCCTTACATCTGAGGACTC
TGCAGTCTATTACTGTGCAAGACGCTATTACTACGGTACCGGACCGG
CTGATTGGTACTTCGATGTCTGGGGCACTGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(10B3 chimera N54Q heavy chain, DNA sequence)
SEQ ID NO: 51
ATGGGATGGAGCTGTATCATCCTCTTCTTGGTAGCAACAGCTACAGG
TGTCCACTCCGAGGTTCAGCTGCAGCAGTCTGGACCTGAACTGGTGA
AGCCTGGGGCTTCAGTGAAGATATCCTGCAAGGCTTCTGGTTACTCA
TTCACTGGCTACTTCATGCACTGGGTGAAGCAGAGCCATGGCAATAT
CCTCGATTGGATTGGAAATATTTATCCTTACCAAGGTGTTTCTAACT
ACAACCAGAGATTCAAGGCCAAGGCCACATTGACTGTAGACAAGTCC
TCTAGTACAGCCTACATGGAGCTCCGCAGCCTTACATCTGAGGACTC
TGCAGTCTATTACTGTGCAAGACGCTATTACTACGGTACCGGACCGG
CTGATTGGTACTTCGATGTCTGGGGCACTGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(10B3 chimera C91S light chain, DNA sequence)
SEQ ID NO: 52
ATGGGATGGAGCTGTATCATCCTCTTCTTGGTAGCAACAGCTACAGG
TGTCCACTCCGACATCAAGATGACCCAGTCTCCATCTTCCATGTATG
CATCTCTACGAGAGAGAGTCACTATCACTTGCAAGGCGAGTCAGGAC
ATTAATAGCTATTTAAGCTGGTTCCAGCAGAAACCAGGGAAATCTCC
TAAGACCCTAATCTATCGTGCAAACAGATTGGTAGATGGGGTCCCAT
CAAGGTTCAGTGGCAGTGGATCTGGGCAAGATTATTCTCTCACCATC
AGCAGCCTGGAGTATGAAGATATGGGAATTTATTATTGTCTACAGTC
TGATGAATTTCCGCTCACGTTCGGTGCTGGGACCAAGCTGGAGCTGA
AACGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(humanised heavy chain: H2 N54D, DNA sequence)
SEQ ID NO: 53
ATGGGCTGGTCCTGCATCATCCTGTTTCTGGTGGCCACCGCCACCGG
CGTGCACAGCCAGGTGCAGCTGGTGCAGAGCGGCGCAGAGGTGAAGA
AGCCCGGCGCCAGCGTGAAAGTGAGCTGCAAGGCCAGCGGCTACTCC
TTCACCGGCTACTTCATGCACTGGGTGAGGCAGGCTCCCGGCCAGGG
CCTGGAGTGGATGGGCAACATCTACCCCTACGACGGCGTCAGCAACT
ACAACCAGAGGTTCAAGGCCAGGGTGACCATGACCACCGACACCTCT
ACCAGCACCGCCTACATGGAACTGAGGAGCCTGAGGAGCGACGACAC
CGCCGTGTACTACTGCGCCAGGAGGTACTATTACGGCACCGGACCCG
CCGATTGGTACTTCGACGTGTGGGGACAGGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(humanised heavy chain: H2 N54Q, DNA sequence)
SEQ ID NO: 54
ATGGGCTGGTCCTGCATCATCCTGTTTCTGGTGGCCACCGCCACCGG
CGTGCACAGCCAGGTGCAGCTGGTGCAGAGCGGCGCAGAGGTGAAGA
AGCCCGGCGCCAGCGTGAAAGTGAGCTGCAAGGCCAGCGGCTACTCC
TTCACCGGCTACTTCATGCACTGGGTGAGGCAGGCTCCCGGCCAGGG
CCTGGAGTGGATGGGCAACATCTACCCCTACCAGGGCGTCAGCAACT
ACAACCAGAGGTTCAAGGCCAGGGTGACCATGACCACCGACACCTCT
ACCAGCACCGCCTACATGGAACTGAGGAGCCTGAGGAGCGACGACAC
CGCCGTGTACTACTGCGCCAGGAGGTACTATTACGGCACCGGACCCG
CCGATTGGTACTTCGACGTGTGGGGACAGGGGACACTAGTGACCGTG
TCCAGCGCCAGCACCAAGGGCCCCAGCGTGTTCCCCCTGGCCCCCAG
CAGCAAGAGCACCAGCGGCGGCACAGCCGCCCTGGGCTGCCTGGTGA
AGGACTACTTCCCCGAACCGGTGACCGTGTCCTGGAACAGCGGAGCC
CTGACCAGCGGCGTGCACACCTTCCCCGCCGTGCTGCAGAGCAGCGG
CCTGTACAGCCTGAGCAGCGTGGTGACCGTGCCCAGCAGCAGCCTGG
GCACCCAGACCTACATCTGTAACGTGAACCACAAGCCCAGCAACACC
AAGGTGGACAAGAAGGTGGAGCCCAAGAGCTGTGACAAGACCCACAC
CTGCCCCCCCTGCCCTGCCCCCGAGCTGCTGGGAGGCCCCAGCGTGT
TCCTGTTCCCCCCCAAGCCTAAGGACACCCTGATGATCAGCAGAACC
CCCGAGGTGACCTGTGTGGTGGTGGATGTGAGCCACGAGGACCCTGA
GGTGAAGTTCAACTGGTACGTGGACGGCGTGGAGGTGCACAATGCCA
AGACCAAGCCCAGGGAGGAGCAGTACAACAGCACCTACCGGGTGGTG
TCCGTGCTGACCGTGCTGCACCAGGATTGGCTGAACGGCAAGGAGTA
CAAGTGTAAGGTGTCCAACAAGGCCCTGCCTGCCCCTATCGAGAAAA
CCATCAGCAAGGCCAAGGGCCAGCCCAGAGAGCCCCAGGTGTACACC
CTGCCCCCTAGCAGAGATGAGCTGACCAAGAACCAGGTGTCCCTGAC
CTGCCTGGTGAAGGGCTTCTACCCCAGCGACATCGCCGTGGAGTGGG
AGAGCAACGGCCAGCCCGAGAACAACTACAAGACCACCCCCCCTGTG
CTGGACAGCGATGGCAGCTTCTTCCTGTACAGCAAGCTGACCGTGGA
CAAGAGCAGATGGCAGCAGGGCAACGTGTTCAGCTGCTCCGTGATGC
ACGAGGCCCTGCACAATCACTACACCCAGAAGAGCCTGAGCCTGTCC
CCTGGCAAGTGA
(humanised light chain: L2 C91S, DNA sequence)
SEQ ID NO: 55
ATGGGCTGGTCCTGCATCATCCTGTTTCTGGTGGCCACCGCCACCGG
CGTGCACAGCGACATTCAGATGACCCAGAGCCCCAGCTCTCTGAGCG
CCAGCGTGGGCGATAGGGTGACCATCACCTGCAAGGCCAGCCAGGAC
ATCAACAGCTACCTGAGCTGGTTCCAGCAGAAGCCCGGCAAGGCTCC
CAAGAGCCTGATCTACAGGGCCAACAGGCTCGTGGACGGCGTGCCTA
GCAAGTTTAGCGGCAGCGGAAGCGGCACAGACTACACCCTGACCATC
AGCTCCCTGCAGCCCGAGGACTTCGCCACCTACTACTGCCTGCAGAG
CGACGAGTTCCCCCTGACCTTCGGCCAGGGCACCAAACTGGAGATCA
AGCGTACGGTGGCCGCCCCCAGCGTGTTCATCTTCCCCCCCAGCGAT
GAGCAGCTGAAGAGCGGCACCGCCAGCGTGGTGTGTCTGCTGAACAA
CTTCTACCCCCGGGAGGCCAAGGTGCAGTGGAAGGTGGACAATGCCC
TGCAGAGCGGCAACAGCCAGGAGAGCGTGACCGAGCAGGACAGCAAG
GACTCCACCTACAGCCTGAGCAGCACCCTGACCCTGAGCAAGGCCGA
CTACGAGAAGCACAAGGTGTACGCCTGTGAGGTGACCCACCAGGGCC
TGTCCAGCCCCGTGACCAAGAGCTTCAACCGGGGCGAGTGCTGA
(artificial myostatin linear peptide 1)
SEQ ID NO: 56
DFGLDCDEHSTESRGSG
(artificial myostatin linear peptide 3)
SEQ ID NO: 57
SGSGDCDEHSTESRCCRY
(artificial myostatin linear peptide 5)
SEQ ID NO: 58
SGSGHSTESRCCRYPLTV
(artificial myostatin linear peptide 7)
SEQ ID NO: 59
SGSGSRCCRYPLTVDFEA
(artificial myostatin linear peptide 9)
SEQ ID NO: 60
SGSGRYPLTVDFEAFGWD
(artificial myostatin linear peptide 11)
SEQ ID NO: 61
SGSGTVDFEAFGWDWIIA
(artificial myostatin linear peptide 13)
SEQ ID NO: 62
SGSGEAFGWDWIIAPKRY
(artificial myostatin linear peptide 15)
SEQ ID NO: 63
SGSGWDWIIAPKRYKANY
(artificial myostatin linear peptide 17)
SEQ ID NO: 64
SGSGIAPKRYKANYCSGE
(artificial myostatin linear peptide 19)
SEQ ID NO: 65
SGSGRYKANYCSGECEFV
(artificial myostatin linear peptide 21)
SEQ ID NO: 66
SGSGNYCSGECEFVFLQK
(artificial myostatin linear peptide 23)
SEQ ID NO: 67
SGSGGECEFVFLQKYPHT
(artificial myostatin linear peptide 25)
SEQ ID NO: 68
SGSGFVFLQKYPHTHLVH
(artificial myostatin linear peptide 27)
SEQ ID NO: 69
SGSGQKYPHTHLVHQANP
(artificial myostatin linear peptide 29)
SEQ ID NO: 70
SGSGHTHLVHQANPRGSA
(artificial myostatin linear peptide 31)
SEQ ID NO: 71
SGSGVHQANPRGSAGPCC
(artificial myostatin linear peptide 33)
SEQ ID NO: 72
SGSGNPRGSAGPCCTPTK
(artificial myostatin linear peptide 35)
SEQ ID NO: 73
SGSGSAGPCCTPTKMSPI
(artificial myostatin linear peptide 37)
SEQ ID NO: 74
SGSGCCTPTKMSPINMLY
(artificial myostatin linear peptide 39)
SEQ ID NO: 75
SGSGTKMSPINMLYFNGK
(artificial myostatin linear peptide 41)
SEQ ID NO: 76
SGSGPINMLYFNGKEQII
(artificial myostatin linear peptide 43)
SEQ ID NO: 77
SGSGLYFNGKEQIIYGKI
(artificial myostatin linear peptide 45)
SEQ ID NO: 78
SGSGGKEQIIYGKIPAMV
(artificial myostatin linear peptide 47)
SEQ ID NO: 79
SGSGIIYGKIPAMVVDRC
(artificial myostatin linear peptide 49)
SEQ ID NO: 80
SGSGGKIPAMVVDRCGCS
(artificial myostatin linear peptide)
SEQ ID NO: 81
CCTPTKMSPINMLY
(CDRH3 variant Y96L)
SEQ ID NO: 82
RLYYGTGPADWYFDV
(CDRH3 variant G99D)
SEQ ID NO: 83
RYYYDTGPADWYFDV
(CDRH3 variant G99S)
SEQ ID NO: 84
RYYYSTGPADWYFDV
(CDRH3 variant G100A_K)
SEQ ID NO: 85
RYYYGTKPADWYFDV
(CDRH3 variant P100B_F)
SEQ ID NO: 86
RYYYGTGFADWYFDV
(CDRH3 variant P100B_I)
SEQ ID NO: 87
RYYYGTGIADWYFDV
(CDRH3 variant W100E_F)
SEQ ID NO: 88
RYYYGTGPADFYFDV
(CDRH3 variant F100G_N)
SEQ ID NO: 89
RYYYGTGPADWYNDV
(CDRH3 variant F100G_Y)
SEQ ID NO: 90
RYYYGTGPADWYYDV
(CDRH3 variant V102N)
SEQ ID NO: 91
RYYYGTGPADWYFDN
(CDRH3 variant V102S)
SEQ ID NO: 92
RYYYGTGPADWYFDS
(CDRH2 variant G55D)
SEQ ID NO: 93
NIYPYNDVSNYNQRFKA
(CDRH2 variant G55L)
SEQ ID NO: 94
NIYPYNLVSNYNQRFKA
(CDRH2 variant G55S)
SEQ ID NO: 95
NIYPYNSVSNYNQRFKA
(CDRH2 variant G55T)
SEQ ID NO: 96
NIYPYNTVSNYNQRFKA
(CDRH2 variant G55V)
SEQ ID NO: 97
NIYPYNVVSNYNQRFKA
(humanised heavy chain: H2_F100G_Y Fc disabled)
SEQ ID NO: 98
QVQLVQSGAEVKKPGASVKVSCKASGYSFTGYFMHWVRQAPGQGLEW
MGNIYPYNGVSNYNQRFKARVTMTTDTSTSTAYMELRSLRSDDTAVY
YCARRYYYGTGPADWYYDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELAGAPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(humanised heavy chain: H2_G55S-F100G_Y Fc
disabled)
SEQ ID NO: 99
QVQLVQSGAEVKKPGASVKVSCKASGYSFTGYFMHWVRQAPGQGLEW
MGNIYPYNSVSNYNQRFKARVTMTTDTSTSTAYMELRSLRSDDTAVY
YCARRYYYGTGPADWYYDVWGQGTLVTVSSASTKGPSVFPLAPSSKS
TSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPP
CPAPELAGAPSVFLFPPKPKDTLMISRTPEVTCVVVDVSHEDPEVKF
NWYVDGVEVHNAKTKPREEQYNSTYRVVSVLTVLHQDWLNGKEYKCK
VSNKALPAPIEKTISKAKGQPREPQVYTLPPSRDELTKNQVSLTCLV
KGFYPSDIAVEWESNGQPENNYKTTPPVLDSDGSFFLYSKLTVDKSR
WQQGNVFSCSVMHEALHNHYTQKSLSLSPGK
(human acceptor framework for V_{L})
SEQ ID NO: 100
DIQMTQSPSSLSASVGDRVTITCRASQGISNYLAWFQQKPGKAPKSL
IYAASSLQSGVPSKFSGSGSGTDFTLTISSLQPEDFATYYCQQYNSY
PXXXXXXXXXXFGQGTKLEIK
(HexaHisGB1Tev/(D76A) mouse myostatin
polyprotein)
SEQ ID NO: 101
MAAGTAVGAWVLVLSLWGAVVGTHHHHHHDTYKLILNGKTLKGETTT
EAVDAATAEKVFKQYANDNGVDGEWTYDDATKTFTVTEGSENLYFQE
GSEREENVEKEGLCNACAWRQNTRYSRIEAIKIQILSKLRLETAPNI
SKDAIRQLLPRAPPLRELIDQYDVQRADSSDGSLEDDDYHATTETII
TMPTESDFLMQADGKPKCCFFKFSSKIQYNKVVKAQLWIYLRPVKTP
TTVFVQILRLIKPMKDGTRYTGIRSLKLDMSPGTGIWQSIDVKTVLQ
NWLKQPESNLGIEIKALDENGHDLAVTFPGPGEDGLNPFLEVKVTDT
PKRSRRDFGLDCDEHSTESRCCRYPLTVDFEAFGWDWIIAPKRYKAN
YCSGECEFVFLQKYPHTHLVHQANPRGSAGPCCTPTKMSPINMLYFN
GKEQIIYGKIPAMVVDRCGCS
(GB1 tag)
SEQ ID NO: 102
DTYKLILNGKTLKGETTTEAVDAATAEKVFKQYANDNGVDGEWTYDD
ATKTFTVTE
(mouse myostatin polyprotein (D76A))
SEQ ID NO: 103
EGSEREENVEKEGLCNACAWRQNTRYSRIEAIKIQILSKLRLETAPN
ISKDAIRQLLPRAPPLRELIDQYDVQRADSSDGSLEDDDYHATTETI
ITMPTESDFLMQADGKPKCCFFKFSSKIQYNKVVKAQLWIYLRPVKT
PTTVFVQILRLIKPMKDGTRYTGIRSLKLDMSPGTGIWQSIDVKTVL
QNWLKQPESNLGIEIKALDENGHDLAVTFPGPGEDGLNPFLEVKVTD
TPKRSRRDFGLDCDEHSTESRCCRYPLTVDFEAFGWDWIIAPKRYKA
NYCSGECEFVFLQKYPHTHLVHQANPRGSAGPCCTPTKMSPINMLYF
NGKEQIIYGKIPAMVVDRCGCS
(mature myostatin)
SEQ ID NO: 104
DFGLDCDEHSTESRCCRYPLTVDFEAFGWDWIIAPKRYKANYCSGEC
EFVFLQKYPHTHLVHQANPRGSAGPCCTPTKMSPINMLYFNGKEQII
YGKIPAMVVDRCGCS
(Furin expression construct)
SEQ ID NO: 105
MELRPWLLWVVAATGTLVLLAADAQGQKVFTNTWAVRIPGGPAVANS
VARKHGFLNLGQIFGDYYHFWHRGVTKRSLSPHRPRHSRLQREPQVQ
WLEQQVAKRRTKRDVYQEPTDPKFPQQWYLSGVTQRDLNVKAAWAQG
YTGHGIVVSILDDGIEKNHPDLAGNYDPGASFDVNDQDPDPQPRYTQ
MNDNRHGTRCAGEVAAVANNGVCGVGVAYNARIGGVRMLDGEVTDAV
EARSLGLNPNHIHIYSASWGPEDDGKTVDGPARLAEEAFFRGVSQGR
GGLGSIFVWASGNGGREHDSCNCDGYTNSIYTLSISSATQFGNVPWY
SEACSSTLATTYSSGNQNEKQIVTTDLRQKCTESHTGTSASAPLAAG
IIALTLEANKNLTWRDMQHLVVQTSKPAHLNANDWATNGVGRKVSHS
YGYGLLDAGAMVALAQNWTTVAPQRKCIIDILTEPKDIGKRLEVRKT
VTACLGEPNHITRLEHAQARLTLSYNRRGDLAIHLVSPMGTRSTLLA
ARPHDYSADGFNDWAFMTTHSWDEDPSGEWVLEIENTSEANNYGTLT
KFTLVLYGTAPEGLPVPPESSGCKTLTSSQACENLYFQG
(HexaHisGB1Tev/Human Myostatin pro-peptide)
SEQ ID NO: 106
MAAGTAVGAWVLVLSLWGAVVGTHHHHHHDTYKLILNGKTLKGETTT
EAVDAATAEKVFKQYANDNGVDGEWTYDDATKTFTVTEGSENLYFQE
NSEQKENVEKEGLCNACTWRQNTKSSRIEAIKIQILSKLRLETAPNI
SKDVIRQLLPKAPPLRELIDQYDVQRDDSSDGSLEDDDYHATTETII
TMPTESDFLMQVDGKPKCCFFKFSSKIQYNKVVKAQLWIYLRPVETP
TTVFVQILRLIKPMKDGTRYTGIRSLKLDMNPGTGIWQSIDVKTVLQ
NWLKQPESNLGIEIKALDENGHDLAVTFPGPGEDGLNPFLEVKVTDT
PKRSRR
(Tev protease expression construct)
SEQ ID NO: 107
MHGHHHHHHGESLFKGPRDYNPISSTICHLTNESDGHTTSLYGIGFG
PFIITNKHLFRRNNGTLLVQSLHGVFKVKNTTTLQQHLIDGRDMIII
RMPKDFPPFPQKLKFREPQREERICLVTTNFQTKSMSSMVSDTSCTF
PSSDGIFWKHWIQTKDGQCGSPLVSTRDGFIVGIHSASNFTNTNNYF
TSVPKNFMELLTNQEAQQWVSGWRLNADSVLWGGHKVFMVKPEEPFQ
PVKEATQLMNE
(human myostatin pro-peptide)
SEQ ID NO: 108
ENSEQKENVEKEGLCNACTWRQNTKSSRIEAIKIQILSKLRLETAPN
ISKDVIRQLLPKAPPLRELIDQYDVQRDDSSDGSLEDDDYHATTETI
ITMPTESDFLMQVDGKPKCCFFKFSSKIQYNKVVKAQLWIYLRPVET
PTTVFVQILRLIKPMKDGTRYTGIRSLKLDMNPGTGIWQSIDVKTVL
QNWLKQPESNLGIEIKALDENGHDLAVTFPGPGEDGLNPFLEVKVTD
TPKRSRR
(CDRL3 variant C91S)
SEQ ID NO: 109
LQSDEFPLT
(CDRH3 variant F100G_S)
SEQ ID NO: 110
RYYYGTGPADWYSDV
(BMP-1 expression construct)
SEQ ID NO: 111
MPGVARLPLLLGLLLLPRPGRPLDLADYTYDLAEEDDSEPLNYKDPC
KAAAFLGDIALDEEDLRAFQVQQAVDLRRHTARKSSIKAAVPGNTST
PSCQSTNGQPQRGACGRWRGRSRSRRAATSRPERVWPDGVIPFVIGG
NFTGSQRAVFRQAMRHWEKHTCVTFLERTDEDSYIVFTYRPCGCCSY
VGRRGGGPQAISIGKNCDKFGIVVHELGHVVGFWHEHTRPDRDRHVS
IVRENIQPGQEYNFLKMEPQEVESLGETYDFDSIMHYARNTFSRGIF
LDTIVPKYEVNGVKPPIGQRTRLSKGDIAQARKLYKCPACGETLQDS
TGNFSSPEYPNGYSAHMHCVWRISVTPGEKIILNFTSLDLYRSRLCW
YDYVEVRDGFWRKAPLRGRFCGSKLPEPIVSTDSRLWVEFRSSSNWV
GKGFFAVYEAICGGDVKKDYGHIQSPNYPDDYRPSKVCIWRIQVSEG
FHVGLTFQSFEIERHDSCAYDYLEVRDGHSESSTLIGRYCGYEKPDD
IKSTSSRLWLKFVSDGSINKAGFAVNFFKEVDECSRPNRGGCEQRCL
NTLGSYKCSCDPGYELAPDKRRCEAACGGFLTKLNGSITSPGWPKEY
PPNKNCIWQLVAPTQYRISLQFDFFETEGNDVCKYDFVEVRSGLTAD
SKLHGKFCGSEKPEVITSQYNNMRVEFKSDNTVSKKGFKAHFFSEKR
PALQPPRGRPHQLKFRVQKRNRTPQENLYFQGWSHPQFEKGTDTYKL
ILNGKTLKGETTTEAVDAATAEKVFKQYANDNGVDGEWTYDDATKTF
TVTE

1-25. (canceled)

26. An antigen binding protein which specifically binds to myostatin and comprises CDRH3 of SEQ ID NO: 3; or a variant CDRH3 wherein the variant CDRH3 is (i) any one of SEQ ID NOs: 82-92, or 110; or (ii) is SEQ ID NO: 3 with any one of the following Kabat substitutions V102Y, V102H, V102I, V102S, V102D or V102G.

27. The antigen binding protein according to claim 26 which further comprises one or more or all CDRs selected from: CDRH1 (SEQ ID NO: 1) or a variant CDRH1; CDRH2 (SEQ ID NO: 2) or a variant CDRH2; CDRL1 (SEQ ID NO: 4) or a variant CDRL1; CDRL2 (SEQ ID NO: 5) or a variant CDRL2; and CDRL3 (SEQ ID NO: 6 or 109) or a variant CDRL3 wherein the CDRH1 variant is CDRH1 of SEQ ID NO:1 and which further comprises one or more or all the following Kabat substitutions: Y32I, Y32H, Y32F, Y32T, Y32N, Y32C, Y32E, Y32D, F33Y, F33A, F33W, F33G, F33T, F33L, F33V, M34I, M34V, M34W, H35E, H35N, H35Q, H35S, H35Y, H35T, and/or the CDRH2 variant wherein the variant CDRH2 is (i) any one of SEQ ID NOs: 93-97; or (ii) the CDRH2 variant wherein the variant is CDRH2 of SEQ ID NO: 2 which further comprises one or more or all the following Kabat substitutions N50R, N50E, N50W, N50Y, N500, N50Q, N50V, N50L, N50K, N50A, I51L, I51V, I51T, I51S, I51N, Y52D, Y52L, Y52N, Y52S, Y53A, Y53G, Y53S, Y53K, Y53T, Y53N, N54S, N54T, N54K, N54D, N54G, G55D, G55L, G55S, G55T, G55V, V56Y, V56R, V56E, V56D, V56G, V56S, V56A, N58K, N58T, N58S, N58D, N58R, N58G, N58F, N58Y and/or the CDRL1 variant wherein the CDRL1 variant is SEQ ID NO: 4 which further comprises one or more or all the following Kabat substitutions D28N, D28S, D28E, D28T, I29V, N30D, N30L, N30Y, N30V, N30I, N30S, N30F, N30H, N30G, N30T, S31N, S31T, S31K, S31G, Y32F, Y32N, Y32A, Y32H, Y32S, Y32R, L33M, L33V, L33I, L33F, S34A, S34G, S34N, S34H, S34V, S34F, and/or the CDRL2 variant wherein the CDRL2 variant is SEQ ID NO:5 which further comprises one or more or all the following Kabat substitutions A51T, A51G or A51V and/or the CDRL3 variant wherein the CDRL3 variant is SEQ ID NO: 6 or is SEQ ID NO: 109 or is SEQ ID NO: 109 which may comprise one or more or all the following Kabat substitutions L89Q, L89S, L89G, L89F, Q90N, Q90H, 591N, S91F, S91G, S91R, S91D, S91H, S91T, S91Y, S91V, D92N, D92Y, D92W, D92T, D92S, D92R, D92Q, D92H, D92A, E93N, E93G, E93H, E93T, E93S, E93R, E93A, F94D, F94Y, F94T, F94V, F94L, F94H, F94N, F94I, F94W, F94P, F94S, L96P, L96Y, L96R, L96I, L96W, L96F.

28. The antigen binding protein according to claim 26, wherein CDRH3 is SEQ ID NO: 90; and/or CDRH2 is SEQ ID NO: 95; and/or CDRL3 is SEQ ID NO: 109.

29. An antigen binding protein which specifically binds to myostatin and comprises CDRH3 of the variable domain sequence of SEQ ID NO: 7 wherein the CDR is determined by any one of Kabat, Chothia, AbM or Contact CDR residues.

30. The antigen binding protein according to claim 29 which further comprises one or more or all of the CDRs as determined by any one of Kabat, Chothia, AbM or Contact CDR residues selected from CDRH1, or CDRH2 of the variable domain sequence of SEQ ID NO: 7; or CDRL1, CDRL2 or CDRL3 of the variable domain sequence of SEQ ID NO: 8.

31. An antigen binding protein which specifically binds to myostatin and comprises a CDRH3 with Kabat residues 95-101 of SEQ ID NO: 7.

32. The antigen binding protein according to claim 31 which further comprises one or more or all minimum binding units selected from: H1 comprising Kabat residues 31-32 of SEQ ID NO: 7; H2 comprising Kabat residues 52-56 of SEQ ID NO 7; L1 comprising Kabat residues 30-34 of SEQ ID NO: 8; L2 comprising Kabat residues 50-55 of SEQ ID NO: 8; and L3 comprising Kabat residues 89-96 of SEQ ID NO: 8.

33. The antigen binding protein according to claim 26 which comprises a variable heavy chain region and/or a variable light chain region comprising any one or a combination of Kabat amino acid residues selected from:
(a) S or T at position 28;
(b) T or Q at position 105;
(c) V, I or G at position 2;
(d) L or V at position 4;
(e) L, I, M or V at position 20;
(f) C at position 22;
(g) T, A, V, G or S at position 24;
(h) G at position 26;
(i) I, F, L or S at position 29;
(j) W at position 36;
(k) W or Y at position 47;
(l) I, M, V or L at position 48;
(m) I, L, F, M or V at position 69;
(n) A, L, V, Y or F at position 78;
(o) L or M at position 80;
(p) Y or F at position 90;
(q) C at position 92; and/or
(r) R, K, G, S, H or N at position 94;
of the variable heavy chain;
and/or
(a) R or G at position 16;
(b) Y or F at position 71;
(c) A or Q at position 100;
(d) I, L or V at position 2;
(e) V, Q, L or E at position 3;
(f) M or L at position 4;
(g) C at position 23;
(h) W at position 35;
(i) Y, L or F at position 36;
(j) S, L, R or V at position 46;
(k) Y, H, F or K at position 49;
(l) C at position 88; and/or
(m) F at position 98;
of the variable light chain.

34. The antigen binding protein according to claim 33 which comprises a variable heavy chain region and/or a variable light chain region comprising any one or a combination of Kabat amino acid residues selected from:
(a) S at position 28;
(b) Q at position 105;
(c) V at position 2;
(d) L at position 4;
(e) V at position 20;
(f) C at position 22;
(g) A at position 24;
(h) G at position 26;
(i) F at position 29;
(j) W at position 36;
(k) W at position 47;
(l) M at position 48;
(m) M at position 69;
(n) A at position 78;
(o) M at position 80;
(p) Y at position 90;
(q) C at position 92; and/or
(r) R at position 94;
of the variable heavy chain;
and/or
(a) G at position 16;
(b) Y at position 71;
(c) Q at position 100;
(d) I at position 2;
(e) Q at position 3;
(f) M at position 4;
(g) C at position 23;
(h) W at position 35;
(i) F at position 36;
(j) S at position 46;
(k) Y at position 49;
(l) C at position 88; and/or
(m) F at position 98;
of the variable light chain.

35. The antigen binding protein according to claim 26 which further comprises a heavy chain variable region acceptor antibody framework having 75% or greater sequence identity to the framework regions as shown in SEQ ID NO: 10; or a light chain variable domain acceptor antibody framework having 75% or greater sequence identity to the framework regions as shown in SEQ ID NO: 11.

36. An antigen binding protein which specifically binds to myostatin and comprises:
(i) a heavy chain variable region selected from SEQ ID NO: 7 or SEQ ID NO: 25; and/or a light chain variable region selected from SEQ ID NO: 8 or SEQ ID NO: 21; or a variant heavy chain variable region or light chain variable region with 90% or greater sequence identity to the heavy chain variable region of SEQ ID NO's: 7 or 25 or light chain variable region of SEQ ID NO's: 8 or 21.
(ii) a heavy chain of SEQ ID NO: 26; and/or a light chain selected from SEQ ID NO: 27 or SEQ ID NO: 37; or a variant heavy chain variable region or light chain variable region with 90% or greater sequence identity to the heavy chain variable region of SEQ ID NO: 26 or light chain variable region of SEQ ID NO's: 27 or 37.

37. An antigen binding protein according to claim 26 which specifically binds to myostatin and comprises:
(i) a heavy chain variable region selected from SEQ ID NO: 7 or SEQ ID NO: 25; and/or a light chain variable region selected from SEQ ID NO: 8 or SEQ ID NO: 21; or a variant heavy chain variable region or light chain variable region with 75% or greater sequence identity to the heavy chain variable region of SEQ ID NO's: 7 or 25 or light chain variable region of SEQ ID NO's: 8 or 21.
(ii) a heavy chain of SEQ ID NO: 26; and/or a light chain selected from SEQ ID NO: 27 or SEQ ID NO: 37; or a variant heavy chain variable region or light chain variable region with 75% or greater sequence identity to the heavy chain variable region of SEQ ID NO: 26 or light chain variable region of SEQ ID NO's: 27 or 37.

38. The antigen binding protein according to claim 36, wherein the following Kabat substitutions are present:
(i) Y96L, G99D, G99S, G100A_K, P100B_F, P100B_I, W100E_F, F100G_N, F100G_S, F 100G_Y, V102N, or V102S in the heavy chain variable region or heavy chain; and/or
(ii) G55D, G55L, G55S, G55T or G55V, in the heavy chain variable region or heavy chain; and/or
(iii) C91S in the light chain variable region or light chain.

39. The antigen binding protein according to any preceding claim wherein the antigen binding protein comprises a VH domain of SEQ.ID.NO: 14.

40. The antigen binding protein according to claim 39 which further comprises a VL domain of SEQ.ID.NO: 17 or SEQ ID NO: 24.

41. An antigen binding protein which specifically binds to myostatin and comprises a heavy chain of SEQ ID NO: 30.

42. An antigen binding protein according to claim 41 which further comprises a light chain of SEQ ID NO: 33 or 40.

43. An antigen binding protein which specifically binds to myostatin and comprises a heavy chain of SEQ ID NO: 98 or SEQ ID NO: 99.

44. An antigen binding protein according to claim 43 which further comprises a light chain of SEQ ID NO: 40.

45. The antigen binding protein according to claim 26 which further comprises a constant region.

46. The antigen binding protein according to any claim 45 wherein the antigen binding protein is an antibody.

47. The antibody of claim 46 wherein the antibody is a monoclonal antibody.

48. The antibody of claim 47 wherein the antibody is humanised.

49. The antibody according to claim 46 wherein the antibody is Fc disabled.

50. A nucleic acid molecule which encodes an antigen binding protein as defined in claim 26.

51. The nucleic acid molecule according to claim 50 which comprises:
(i) a DNA sequence of SEQ ID NO: 41 which encodes a heavy chain; and/or a DNA sequence selected from SEQ ID NO: 42 or 52 which encodes a light chain; or a variant DNA sequence which codes for a heavy chain or light chain with 75% or greater identity to SEQ ID NO's: 41, 42 or 52; or
(ii) a DNA sequence selected from any one of SEQ ID NO: 43, 44 or 45 which encodes a heavy chain; and/or a DNA sequence selected from any one of SEQ ID NO: 46, 47, 48, 49 or 55 which encodes a light chain; or a variant DNA sequence with 75% or greater identity to SEQ ID NO's:43, 44, 45, 46, 47, 48, 49 or 55.

52. An expression vector comprising a nucleic acid molecule as defined in claim 50.

53. A recombinant host cell comprising an expression vector as defined in claim 52.

54. A method for the production of an antigen binding protein as defined in claim 26 which method comprises the step of culturing a host cell as defined in claim 53 and recovering the antigen binding protein.

55. A pharmaceutical composition comprising an antigen binding protein as defined in claim 26 and a pharmaceutically acceptable carrier.

56. A method of treating a subject afflicted with a disease which reduces any one or a combination of muscle mass, muscle strength and muscle function, which method comprises the step of administering an antigen binding protein as defined in claim 26 or the composition of claim 55.

57. A method of treating a subject afflicted with sarcopenia, cachexia, muscle-wasting, disuse muscle atrophy, HIV, AIDS, cancer, surgery, burns, trauma or injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, multiple sclerosis (MS), stroke, muscular dystrophy, motor neuron neuropathy, amyotrophic lateral sclerosis (ALS), Parkinson's disease, osteoporosis, osteoarthritis, fatty acid liver disease, liver cirrhosis, Addison's disease, Cushing's syndrome, acute respiratory distress syndrome, steroid induced muscle wasting, myositis or scoliosis, which method comprises the step of administering an antigen binding protein as defined in claim 26 or the composition of claim 55.

58. A method of increasing muscle mass, increasing muscle strength, and/or improving muscle function in a subject which method comprises the step of administering an antigen binding protein as defined in claim 26 or the composition of claim 55.


SEQUENCE LISTING (111 sequences)

SEQ ID NO: 1 - protein, 5 aa, Mus musculus
GYFMH

SEQ ID NO: 2 - protein, 17 aa, Mus musculus
NIYPYNGVSNYNQRFKA

SEQ ID NO: 3 - protein, 15 aa, Mus musculus
RYYYGTGPADWYFDV

SEQ ID NO: 4 - protein, 11 aa, Mus musculus
KASQDINSYLS

SEQ ID NO: 5 - protein, 7 aa, Mus musculus
RANRLVD

SEQ ID NO: 6 - protein, 9 aa, Mus musculus
LQCDEFPLT

SEQ ID NO: 7 - protein, 124 aa, Mus musculus
EVQLQQSGPELVKPGASVKISCKASGYSFTGYFMHWVKQSHGNILDWIGNIYPYNGVSNYNQRFKAKATLTVDKSSSTAYMELRSLTSEDSAVYYCARRYYYGTGPADWYFDVWGTGTTVTVSS

SEQ ID NO: 8 - protein, 107 aa, Mus musculus
DIKMTQSPSSMYASLRERVTITCKASQDINSYLSWFQQKPGKSPKTLIYRANRLVDGVPSRFSGSGSGQDYSLTISSLEYEDMGIYYCLQCDEFPLTFGAGTKLELK

SEQ ID NO: 9 - protein, 19 aa, Artificial Sequence
  artificial signal sequence
MGWSCIILFLVATATGVHS

SEQ ID NO: 10 - protein, 119 aa, Artificial Sequence
  human acceptor framework for VH
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARXXXXXXXXXXWGQGTMVTVSS

SEQ ID NO: 11 - protein, 108 aa, Artificial Sequence
  human acceptor framework for VL
DIQMTQSPSSLSASVGDRVTITCRASQGISNYLAWFQQKPGKAPKSLIYAASSLQSGVPSKFSGSGSGTDFTLTISSLQPEDFATYYCXXXXXXXXXXFGQGTKLEIK

SEQ ID NO: 12 - protein, 124 aa, Artificial Sequence
  humanised VH H0
QVQLVQSGAEVKKPGASVKVSCKASGYTFTGYFMHWVRQAPGQGLEWMGNIYPYNGVSNYNQRFKARVTMTTDTSTSTAYMELRSLRSDDTAVYYCARRYYYGTGPADWYFDVWGQGTLVTVSS

SEQ ID NO: 13 - protein, 124 aa, Artificial Sequence
  humanised VH H1
QVQLVQSGAEVKKPGASVKVSCKASGYTFTGYFMHWVRQAPGQGLEWMGNIYPYNGVSNYNQRFKARVTMTTDTSTSTAYMELRSLRSDDTAVYYCARRYYYGTGPADWYFDVWGTGTLVTVSS

SEQ ID NO: 14 - protein, 124 aa, Artificial Sequence
  humanised VH H2
QVQLVQSGAEVKKPGASVKVSCKASGYSFTGYFMHWVRQAPGQGLEWMGNIYPYNGVSNYNQRFKARVTMTTDTSTSTAYMELRSLRSDDTAVYYCARRYYYGTGPADWYFDVWGQGTLVTVSS

SEQ ID NO: 15 - protein, 107 aa, Artificial Sequence
  humanised VL L0
DIQMTQSPSSLSASVGDRVTITCKASQDINSYLSWFQQKPGKAPKSLIYRANRLVDGVPSKFSGSGSGTDFTLTISSLQPEDFATYYCLQCDEFPLTFGQGTKLEIK

SEQ ID NO: 16 - protein, 107 aa, Artificial Sequence
  humanised VL L1
DIQMTQSPSSLSASVRDRVTITCKASQDINSYLSWFQQKPGKAPKSLIYRANRLVDGVPSKFSGSGSGTDFTLTISSLQPEDFATYYCLQCDEFPLTFGQGTKLEIK

SEQ ID NO: 17 - protein, 107 aa, Artificial Sequence
  humanised VL L2
DIQMTQSPSSLSASVGDRVTITCKASQDINSYLSWFQQKPGKAPKSLIYRANRLVDGVPSKFSGSGSGTDYTLTISSLQPEDFATYYCLQCDEFPLTFGQGTKLEIK

SEQ ID NO: 18 - protein, 107 aa, Artificial Sequence
  humanised VL L3
DIQMTQSPSSLSASVGDRVTITCKASQDINSYLSWFQQKPGKAPKSLIYRANRLVDGVPSKFSGSGSGTDFTLTISSLQPEDFATYYCLQCDEFPLTFGAGTKLEIK

SEQ ID NO: 19 - protein, 124 aa, Artificial Sequence
  10B3 chimera VH N54D
EVQLQQSGPELVKPGASVKISCKASGYSFTGYFMHWVKQSHGNILDWIGNIYPYDGVSNYNQRFKAKATLTVDKSSSTAYMELRSLTSEDSAVYYCARRYYYGTGPADWYFDVWGTGTLVTVSS

SEQ ID NO: 20 - protein, 124 aa, Artificial Sequence
  10B3 chimera VH N54Q
EVQLQQSGPELVKPGASVKISCKASGYSFTGYFMHWVKQSHGNILDWIGNIYPYQGVSNYNQRFKAKATLTVDKSSSTAYMELRSLTSEDSAVYYCARRYYYGTGPADWYFDVWGTGTLVTVSS

SEQ ID NO: 21 - protein, 107 aa, Artificial Sequence
  10B3 chimera VL C91S
DIKMTQSPSSMYASLRERVTITCKASQDINSYLSWFQQKPGKSPKTLIYRANRLVDGVPSRFSGSGSGQDYSLTISSLEYEDMGIYYCLQSDEFPLTFGAGTKLELK

SEQ ID NO: 22 - protein, 124 aa, Artificial Sequence
  humanised VH H2 N54D
QVQLVQSGAEVKKPGASVKVSCKASGYSFTGYFMHWVRQAPGQGLEWMGNIYPYDGVSNYNQRFKARVTMTTDTSTSTAYMELRSLRSDDTAVYYCARRYYYGTGPADWYFDVWGQGTLVTVSS

SEQ ID NO: 23 - protein, 124 aa, Artificial Sequence
  humanised VH H2 N54Q
QVQLVQSGAEVKKPGASVKVSCKASGYSFTGYFMHWVRQAPGQGLEWMGNIYPYQGVSNYNQRFKARVTMTTDTSTSTAYMELRSLRSDDTAVYYCARRYYYGTGPADWYFDVWGQGTLVTVSS

SEQ ID NO: 24 - protein, 107 aa, Artificial Sequence
  humanised VL L2 C91S
DIQMTQSPSSLSASVGDRVTITCKASQDINSYLSWFQQKPGKAPKSLIYRANRLVDGVPSKFSGSGSGTDYTLTISSLQPEDFATYYCLQSDEFPLTFGQGTKLEIK

SEQ ID NO: 25 - protein, 124 aa, Artificial Sequence
  10B3 chimera VH
EVQLQQSGPELVKPGASVKISCKASGYSFTGYFMHWVKQSHGNILDWIGNIYPYNGVSNYNQRFKAKATLTVDKSSSTAYMELRSLTSEDSAVYYCARRYYYGTGPADWYFDVWGTGTLVTVSS

SEQ ID NO: 26 - protein, 454 aa, Artificial Sequence
  10B3 chimera heavy chain
EVQLQQSGPELVKPGASVKISCKASGYSFTGYFMHWVKQSHGNILDWIGNIYPYNGVSNYNQRFKAKATLTVDKSSSTAYMELRSLTSEDSAVYYCARRYYYGTGPADWYFDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Artificial Sequence
  10B3 chimera light chain
DIKMTQSPSSMYASLRERVTITCKASQDINSYLSWFQQKPGKSPKTLIYRANRLVDGVPSRFSGSGSGQDYSLTISSLEYEDMGIYYCLQCDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 454 aa, Artificial Sequence
  humanised heavy chain H0
QVQLVQSGAEVKKPGASVKVSCKASGYTFTGYFMHWVRQAPGQGLEWMGNIYPYNGVSNYNQRFKARVTMTTDTSTSTAYMELRSLRSDDTAVYYCARRYYYGTGPADWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 454 aa, Artificial Sequence
  humanised heavy chain H1
QVQLVQSGAEVKKPGASVKVSCKASGYTFTGYFMHWVRQAPGQGLEWMGNIYPYNGVSNYNQRFKARVTMTTDTSTSTAYMELRSLRSDDTAVYYCARRYYYGTGPADWYFDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 454 aa, Artificial Sequence
  humanised heavy chain H2
QVQLVQSGAEVKKPGASVKVSCKASGYSFTGYFMHWVRQAPGQGLEWMGNIYPYNGVSNYNQRFKARVTMTTDTSTSTAYMELRSLRSDDTAVYYCARRYYYGTGPADWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, Artificial Sequence
  humanised light chain L0
DIQMTQSPSSLSASVGDRVTITCKASQDINSYLSWFQQKPGKAPKSLIYRANRLVDGVPSKFSGSGSGTDFTLTISSLQPEDFATYYCLQCDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence
  humanised light chain L1
DIQMTQSPSSLSASVRDRVTITCKASQDINSYLSWFQQKPGKAPKSLIYRANRLVDGVPSKFSGSGSGTDFTLTISSLQPEDFATYYCLQCDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 214 aa, Artificial Sequence
  humanised light chain L2
DIQMTQSPSSLSASVGDRVTITCKASQDINSYLSWFQQKPGKAPKSLIYRANRLVDGVPSKFSGSGSGTDYTLTISSLQPEDFATYYCLQCDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 214 aa, Artificial Sequence
  humanised light chain L3
DIQMTQSPSSLSASVGDRVTITCKASQDINSYLSWFQQKPGKAPKSLIYRANRLVDGVPSKFSGSGSGTDFTLTISSLQPEDFATYYCLQCDEFPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 454 aa, Artificial Sequence
  10B3 chimera N54D heavy chain
EVQLQQSGPELVKPGASVKISCKASGYSFTGYFMHWVKQSHGNILDWIGNIYPYDGVSNYNQRFKAKATLTVDKSSSTAYMELRSLTSEDSAVYYCARRYYYGTGPADWYFDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 454 aa, Artificial Sequence
  10B3 chimera N54Q heavy chain
EVQLQQSGPELVKPGASVKISCKASGYSFTGYFMHWVKQSHGNILDWIGNIYPYQGVSNYNQRFKAKATLTVDKSSSTAYMELRSLTSEDSAVYYCARRYYYGTGPADWYFDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, Artificial Sequence
  10B3 chimera C91S light chain
DIKMTQSPSSMYASLRERVTITCKASQDINSYLSWFQQKPGKSPKTLIYRANRLVDGVPSRFSGSGSGQDYSLTISSLEYEDMGIYYCLQSDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 454 aa, Artificial Sequence
  humanised heavy chain H2 N54D
QVQLVQSGAEVKKPGASVKVSCKASGYSFTGYFMHWVRQAPGQGLEWMGNIYPYDGVSNYNQRFKARVTMTTDTSTSTAYMELRSLRSDDTAVYYCARRYYYGTGPADWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 454 aa, Artificial Sequence
  humanised heavy chain H2 N54Q
QVQLVQSGAEVKKPGASVKVSCKASGYSFTGYFMHWVRQAPGQGLEWMGNIYPYQGVSNYNQRFKARVTMTTDTSTSTAYMELRSLRSDDTAVYYCARRYYYGTGPADWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  humanised light chain L2 C91S
DIQMTQSPSSLSASVGDRVTITCKASQDINSYLSWFQQKPGKAPKSLIYRANRLVDGVPSKFSGSGSGTDYTLTISSLQPEDFATYYCLQSDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 1422 bp, Artificial Sequence
  10B3 chimera heavy chain
atgggatggagctgtatcatcctcttcttggtagcaacagctacaggtgtccactccgaggttcagctgcagcagtctggacctgaactggtgaagcctggggcttcagtgaagatatcctgcaaggcttctggttactcattcactggctacttcatgcactgggtgaagcagagccatggcaatatcctcgattggattggaaatatttatccttacaatggtgtttctaactacaaccagagattcaaggccaaggccacattgactgtagacaagtcctctagtacagcctacatggagctccgcagccttacatctgaggactctgcagtctattactgtgcaagacgctattactacggtaccggaccggctgattggtacttcgatgtctggggcactgggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 42 - DNA, 702 bp, Artificial Sequence
  10B3 chimera light chain
atgggatggagctgtatcatcctcttcttggtagcaacagctacaggtgtccactccgacatcaagatgacccagtctccatcttccatgtatgcatctctacgagagagagtcactatcacttgcaaggcgagtcaggacattaatagctatttaagctggttccagcagaaaccagggaaatctcctaagaccctaatctatcgtgcaaacagattggtagatggggtcccatcaaggttcagtggcagtggatctgggcaagattattctctcaccatcagcagcctggagtatgaagatatgggaatttattattgtctacagtgtgatgaatttccgctcacgttcggtgctgggaccaagctggagctgaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 43 - DNA, 1422 bp, Artificial Sequence
  humanised heavy chain H0
atgggctggtcctgcatcatcctgtttctggtggccaccgccaccggcgtgcacagccaggtgcagctggtgcagagcggcgcagaggtgaagaagcccggcgccagcgtgaaagtgagctgcaaggccagcggctacaccttcaccggctacttcatgcactgggtgaggcaggctcccggccagggcctggagtggatgggcaacatctacccctacaacggcgtcagcaactacaaccagaggttcaaggccagggtgaccatgaccaccgacacctctaccagcaccgcctacatggaactgaggagcctgaggagcgacgacaccgccgtgtactactgcgccaggaggtactattacggcaccggacccgccgattggtacttcgacgtgtggggacaggggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 44 - DNA, 1422 bp, Artificial Sequence
  humanised heavy chain H1
atgggctggtcctgcatcatcctgtttctggtggccaccgccaccggcgtgcacagccaggtgcagctggtgcagagcggcgcagaggtgaagaagcccggcgccagcgtgaaagtgagctgcaaggccagcggctacaccttcaccggctacttcatgcactgggtgaggcaggctcccggccagggcctggagtggatgggcaacatctacccctacaacggcgtcagcaactacaaccagaggttcaaggccagggtgaccatgaccaccgacacctctaccagcaccgcctacatggaactgaggagcctgaggagcgacgacaccgccgtgtactactgcgccaggaggtactattacggcaccggacccgccgattggtacttcgacgtgtggggaacggggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 45 - DNA, 1422 bp, Artificial Sequence
  humanised heavy chain H2
atgggctggtcctgcatcatcctgtttctggtggccaccgccaccggcgtgcacagccaggtgcagctggtgcagagcggcgcagaggtgaagaagcccggcgccagcgtgaaagtgagctgcaaggccagcggctactccttcaccggctacttcatgcactgggtgaggcaggctcccggccagggcctggagtggatgggcaacatctacccctacaacggcgtcagcaactacaaccagaggttcaaggccagggtgaccatgaccaccgacacctctaccagcaccgcctacatggaactgaggagcctgaggagcgacgacaccgccgtgtactactgcgccaggaggtactattacggcaccggacccgccgattggtacttcgacgtgtggggacaggggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 46 - DNA, 702 bp, Artificial Sequence
  humanised light chain L0
atgggctggtcctgcatcatcctgtttctggtggccaccgccaccggcgtgcacagcgacattcagatgacccagagccccagctctctgagcgccagcgtgggcgatagggtgaccatcacctgcaaggccagccaggacatcaacagctacctgagctggttccagcagaagcccggcaaggctcccaagagcctgatctacagggccaacaggctcgtggacggcgtgcctagcaagtttagcggcagcggaagcggcacagacttcaccctgaccatcagctccctgcagcccgaggacttcgccacctactactgcctgcagtgcgacgagttccccctgaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 47 - DNA, 702 bp, Artificial Sequence
  humanised light chain L1
atgggctggtcctgcatcatcctgtttctggtggccaccgccaccggcgtgcacagcgacattcagatgacccagagccccagctctctgagcgccagcgtgcgcgatagggtgaccatcacctgcaaggccagccaggacatcaacagctacctgagctggttccagcagaagcccggcaaggctcccaagagcctgatctacagggccaacaggctcgtggacggcgtgcctagcaagtttagcggcagcggaagcggcacagacttcaccctgaccatcagctccctgcagcccgaggacttcgccacctactactgcctgcagtgcgacgagttccccctgaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 48 - DNA, 702 bp, Artificial Sequence
  humanised light chain L2
atgggctggtcctgcatcatcctgtttctggtggccaccgccaccggcgtgcacagcgacattcagatgacccagagccccagctctctgagcgccagcgtgggcgatagggtgaccatcacctgcaaggccagccaggacatcaacagctacctgagctggttccagcagaagcccggcaaggctcccaagagcctgatctacagggccaacaggctcgtggacggcgtgcctagcaagtttagcggcagcggaagcggcacagactacaccctgaccatcagctccctgcagcccgaggacttcgccacctactactgcctgcagtgcgacgagttccccctgaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 49 - DNA, 702 bp, Artificial Sequence
  humanised light chain L3
atgggctggtcctgcatcatcctgtttctggtggccaccgccaccggcgtgcacagcgacattcagatgacccagagccccagctctctgagcgccagcgtgggcgatagggtgaccatcacctgcaaggccagccaggacatcaacagctacctgagctggttccagcagaagcccggcaaggctcccaagagcctgatctacagggccaacaggctcgtggacggcgtgcctagcaagtttagcggcagcggaagcggcacagacttcaccctgaccatcagctccctgcagcccgaggacttcgccacctactactgcctgcagtgcgacgagttccccctgaccttcggcgcgggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 50 - DNA, 1422 bp, Artificial Sequence
  10B3 chimera N54D heavy chain
atgggatggagctgtatcatcctcttcttggtagcaacagctacaggtgtccactccgaggttcagctgcagcagtctggacctgaactggtgaagcctggggcttcagtgaagatatcctgcaaggcttctggttactcattcactggctacttcatgcactgggtgaagcagagccatggcaatatcctcgattggattggaaatatttatccttacgatggtgtttctaactacaaccagagattcaaggccaaggccacattgactgtagacaagtcctctagtacagcctacatggagctccgcagccttacatctgaggactctgcagtctattactgtgcaagacgctattactacggtaccggaccggctgattggtacttcgatgtctggggcactgggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 51 - DNA, 1422 bp, Artificial Sequence
  10B3 chimera N54Q heavy chain
atgggatggagctgtatcatcctcttcttggtagcaacagctacaggtgtccactccgaggttcagctgcagcagtctggacctgaactggtgaagcctggggcttcagtgaagatatcctgcaaggcttctggttactcattcactggctacttcatgcactgggtgaagcagagccatggcaatatcctcgattggattggaaatatttatccttaccaaggtgtttctaactacaaccagagattcaaggccaaggccacattgactgtagacaagtcctctagtacagcctacatggagctccgcagccttacatctgaggactctgcagtctattactgtgcaagacgctattactacggtaccggaccggctgattggtacttcgatgtctggggcactgggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 52 - DNA, 702 bp, Artificial Sequence
  10B3 chimera C91S light chain
atgggatggagctgtatcatcctcttcttggtagcaacagctacaggtgtccactccgacatcaagatgacccagtctccatcttccatgtatgcatctctacgagagagagtcactatcacttgcaaggcgagtcaggacattaatagctatttaagctggttccagcagaaaccagggaaatctcctaagaccctaatctatcgtgcaaacagattggtagatggggtcccatcaaggttcagtggcagtggatctgggcaagattattctctcaccatcagcagcctggagtatgaagatatgggaatttattattgtctacagtctgatgaatttccgctcacgttcggtgctgggaccaagctggagctgaaacgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 53 - DNA, 1422 bp, Artificial Sequence
  humanised heavy chain H2 N54D
atgggctggtcctgcatcatcctgtttctggtggccaccgccaccggcgtgcacagccaggtgcagctggtgcagagcggcgcagaggtgaagaagcccggcgccagcgtgaaagtgagctgcaaggccagcggctactccttcaccggctacttcatgcactgggtgaggcaggctcccggccagggcctggagtggatgggcaacatctacccctacgacggcgtcagcaactacaaccagaggttcaaggccagggtgaccatgaccaccgacacctctaccagcaccgcctacatggaactgaggagcctgaggagcgacgacaccgccgtgtactactgcgccaggaggtactattacggcaccggacccgccgattggtacttcgacgtgtggggacaggggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 54 - DNA, 1422 bp, Artificial Sequence
  humanised heavy chain H2 N54Q
atgggctggtcctgcatcatcctgtttctggtggccaccgccaccggcgtgcacagccaggtgcagctggtgcagagcggcgcagaggtgaagaagcccggcgccagcgtgaaagtgagctgcaaggccagcggctactccttcaccggctacttcatgcactgggtgaggcaggctcccggccagggcctggagtggatgggcaacatctacccctaccagggcgtcagcaactacaaccagaggttcaaggccagggtgaccatgaccaccgacacctctaccagcaccgcctacatggaactgaggagcctgaggagcgacgacaccgccgtgtactactgcgccaggaggtactattacggcaccggacccgccgattggtacttcgacgtgtggggacaggggacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 55 - DNA, 702 bp, Artificial Sequence
  humanised light chain L2 C91S
atgggctggtcctgcatcatcctgtttctggtggccaccgccaccggcgtgcacagcgacattcagatgacccagagccccagctctctgagcgccagcgtgggcgatagggtgaccatcacctgcaaggccagccaggacatcaacagctacctgagctggttccagcagaagcccggcaaggctcccaagagcctgatctacagggccaacaggctcgtggacggcgtgcctagcaagtttagcggcagcggaagcggcacagactacaccctgaccatcagctccctgcagcccgaggacttcgccacctactactgcctgcagagcgacgagttccccctgaccttcggccagggcaccaaactggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgctga

SEQ ID NO: 56 - protein, 17 aa, Artificial Sequence
  artificial myostatin linear peptide 1
DFGLDCDEHSTESRGSG

SEQ ID NO: 57 - protein, 18 aa, Artificial Sequence
  artificial myostatin linear peptide 3
SGSGDCDEHSTESRCCRY

SEQ ID NO: 58 - protein, 18 aa, Artificial Sequence
  artificial myostatin linear peptide 5
SGSGHSTESRCCRYPLTV

SEQ ID NO: 59 - protein, 18 aa, Artificial Sequence
  artificial myostatin linear peptide 7
SGSGSRCCRYPLTVDFEA

SEQ ID NO: 60 - protein, 18 aa, Artificial Sequence
  artificial myostatin linear peptide 9
SGSGRYPLTVDFEAFGWD

SEQ ID NO: 61 - protein, 18 aa, Artificial Sequence
  artificial myostatin linear peptide 11
SGSGTVDFEAFGWDWIIA

SEQ ID NO: 62 - protein, 18 aa, Artificial Sequence
  artificial myostatin linear peptide 13
SGSGEAFGWDWIIAPKRY

SEQ ID NO: 63 - protein, 18 aa, Artificial Sequence
  artificial myostatin linear peptide 15
SGSGWDWIIAPKRYKANY

SEQ ID NO: 64 - protein, 18 aa, Artificial Sequence
  artificial myostatin linear peptide 17
SGSGIAPKRYKANYCSGE

SEQ ID NO: 65 - protein, 18 aa, Artificial Sequence
  artificial myostatin linear peptide 19
SGSGRYKANYCSGECEFV

SEQ ID NO: 66 - protein, 18 aa, Artificial Sequence
  artificial myostatin linear peptide 21
SGSGNYCSGECEFVFLQK

SEQ ID NO: 67 - protein, 18 aa, Artificial Sequence
  artificial myostatin linear peptide 23
SGSGGECEFVFLQKYPHT

SEQ ID NO: 68 - protein, 18 aa, Artificial Sequence
  artificial myostatin linear peptide 25
SGSGFVFLQKYPHTHLVH

SEQ ID NO: 69 - protein, 18 aa, Artificial Sequence
  artificial myostatin linear peptide 27
SGSGQKYPHTHLVHQANP

SEQ ID NO: 70 - protein, 18 aa, Artificial Sequence
  artificial myostatin linear peptide 29
SGSGHTHLVHQANPRGSA

SEQ ID NO: 71 - protein, 18 aa, Artificial Sequence
  artificial myostatin linear peptide 31
SGSGVHQANPRGSAGPCC

SEQ ID NO: 72 - protein, 18 aa, Artificial Sequence
  artificial myostatin linear peptide 33
SGSGNPRGSAGPCCTPTK

SEQ ID NO: 73 - protein, 18 aa, Artificial Sequence
  artificial myostatin linear peptide 35
SGSGSAGPCCTPTKMSPI

SEQ ID NO: 74 - protein, 18 aa, Artificial Sequence
  artificial myostatin linear peptide 37
SGSGCCTPTKMSPINMLY

SEQ ID NO: 75 - protein, 18 aa, Artificial Sequence
  artificial myostatin linear peptide 39
SGSGTKMSPINMLYFNGK

SEQ ID NO: 76 - protein, 18 aa, Artificial Sequence
  artificial myostatin linear peptide 41
SGSGPINMLYFNGKEQII

SEQ ID NO: 77 - protein, 18 aa, Artificial Sequence
  artificial myostatin linear peptide 43
SGSGLYFNGKEQIIYGKI

SEQ ID NO: 78 - protein, 18 aa, Artificial Sequence
  artificial myostatin linear peptide 45
SGSGGKEQIIYGKIPAMV

SEQ ID NO: 79 - protein, 18 aa, Artificial Sequence
  artificial myostatin linear peptide 47
SGSGIIYGKIPAMVVDRC

SEQ ID NO: 80 - protein, 18 aa, Artificial Sequence
  artificial myostatin linear peptide 49
SGSGGKIPAMVVDRCGCS

SEQ ID NO: 81 - protein, 14 aa, Homo sapien
CCTPTKMSPINMLY

SEQ ID NO: 82 - protein, 15 aa, Artificial Sequence
  CDRH3 variant Y96L
RLYYGTGPADWYFDV

SEQ ID NO: 83 - protein, 15 aa, Artificial Sequence
  CDRH3 variant G99D
RYYYDTGPADWYFDV

SEQ ID NO: 84 - protein, 15 aa, Artificial Sequence
  CDRH3 variant G99S
RYYYSTGPADWYFDV

SEQ ID NO: 85 - protein, 15 aa, Artificial Sequence
  CDRH3 variant G100A_K
RYYYGTKPADWYFDV

SEQ ID NO: 86 - protein, 15 aa, Artificial Sequence
  CDRH3 variant P100B_F
RYYYGTGFADWYFDV

SEQ ID NO: 87 - protein, 15 aa, Artificial Sequence
  CDRH3 variant P100B_I
RYYYGTGIADWYFDV

SEQ ID NO: 88 - protein, 15 aa, Artificial Sequence
  CDRH3 variant W100E_F
RYYYGTGPADFYFDV

SEQ ID NO: 89 - protein, 15 aa, Artificial Sequence
  CDRH3 variant F100G_N
RYYYGTGPADWYNDV

SEQ ID NO: 90 - protein, 15 aa, Artificial Sequence
  CDRH3 variant F100G_Y
RYYYGTGPADWYYDV

SEQ ID NO: 91 - protein, 15 aa, Artificial Sequence
  CDRH3 variant V102N
RYYYGTGPADWYFDN

SEQ ID NO: 92 - protein, 15 aa, Artificial Sequence
  CDRH3 variant V102S
RYYYGTGPADWYFDS

SEQ ID NO: 93 - protein, 17 aa, Artificial Sequence
  CDRH2 variant G55D
NIYPYNDVSNYNQRFKA

SEQ ID NO: 94 - protein, 17 aa, Artificial Sequence
  CDRH2 variant G55L
NIYPYNLVSNYNQRFKA

SEQ ID NO: 95 - protein, 17 aa, Artificial Sequence
  CDRH2 variant G55S
NIYPYNSVSNYNQRFKA

SEQ ID NO: 96 - protein, 17 aa, Artificial Sequence
  CDRH2 variant G55T
NIYPYNTVSNYNQRFKA

SEQ ID NO: 97 - protein, 17 aa, Artificial Sequence
  CDRH2 variant G55V
NIYPYNVVSNYNQRFKA

SEQ ID NO: 98 - protein, 454 aa, Artificial Sequence
  humanised heavy chain H2_F100G_Y Fc disabled
QVQLVQSGAEVKKPGASVKVSCKASGYSFTGYFMHWVRQAPGQGLEWMGNIYPYNGVSNYNQRFKARVTMTTDTSTSTAYMELRSLRSDDTAVYYCARRYYYGTGPADWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 454 aa, Artificial Sequence
  humanised heavy chain H2_G55S-F100G_Y Fc disabled
QVQLVQSGAEVKKPGASVKVSCKASGYSFTGYFMHWVRQAPGQGLEWMGNIYPYNSVSNYNQRFKARVTMTTDTSTSTAYMELRSLRSDDTAVYYCARRYYYGTGPADWYYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 115 aa, Artificial Sequence
  human acceptor framework for VL
DIQMTQSPSSLSASVGDRVTITCRASQGISNYLAWFQQKPGKAPKSLIYAASSLQSGVPSKFSGSGSGTDFTLTISSLQPEDFATYYCQQYNSYPXXXXXXXXXXFGQGTKLEIK

SEQ ID NO: 101 - protein, 444 aa, Artificial Sequence
  HexaHisGB1Tev/(D76A) mouse myostatin polyprotein
MAAGTAVGAWVLVLSLWGAVVGTHHHHHHDTYKLILNGKTLKGETTTEAVDAATAEKVFKQYANDNGVDGEWTYDDATKTFTVTEGSENLYFQEGSEREENVEKEGLCNACAWRQNTRYSRIEAIKIQILSKLRLETAPNISKDAIRQLLPRAPPLRELIDQYDVQRADSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 102 - protein, 56 aa, Artificial Sequence
  GB1 tag
DTYKLILNGKTLKGETTTEAVDAATAEKVFKQYANDNGVDGEWTYDDATKTFTVTE

SEQ ID NO: 103 - protein, 351 aa, Artificial Sequence
  mouse myostatin polyprotein (D76A)
EGSEREENVEKEGLCNACAWRQNTRYSRIEAIKIQILSKLRLETAPNISKDAIRQLLPRAPPLRELIDQYDVQRADSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 104 - protein, 109 aa, Homo sapien
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 105 - protein, 603 aa, Artificial Sequence
  Furin expression construct
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACENLYFQG

SEQ ID NO: 106 - protein, 335 aa, Artificial Sequence
  HexaHisGB1Tev/human myostatin pro-peptide
MAAGTAVGAWVLVLSLWGAVVGTHHHHHHDTYKLILNGKTLKGETTTEAVDAATAEKVFKQYANDNGVDGEWTYDDATKTFTVTEGSENLYFQENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 107 - protein, 246 aa, Artificial Sequence
  Tev protease expression construct
MHGHHHHHHGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 108 - protein, 242 aa, Homo sapien
ENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 109 - protein, 9 aa, Artificial Sequence
  CDRL3 variant C91S
LQSDEFPLT

SEQ ID NO: 110 - protein, 15 aa, Artificial Sequence
  CDRH3 variant Y100G_S
RYYYGTGPADWYSDV

SEQ ID NO: 111 - protein, 803 aa, Artificial Sequence
  BMP-1 expression construct
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSEKRPALQPPRGRPHQLKFRVQKRNRTPQENLYFQGWSHPQFEKGTDTYKLILNGKTLKGETTTEAVDAATAEKVFKQYANDNGVDGEWTYDDATKTFTVTE